SEQUENCE LISTING (136 sequences)

SEQ ID NO: 1-8 - skipped (8 entries)

SEQ ID NO: 9 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 10 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 11 - protein, 100 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 12 - DNA, 1539 bp, Homo sapiens
  source 1..1539: mol_type = genomic DNA
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 13 - DNA, 345 bp, Homo sapiens
  source 1..345: mol_type = genomic DNA
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 14-19 - skipped (6 entries)

SEQ ID NO: 20 - protein, 4 aa, synthetic construct
GGGG

SEQ ID NO: 21 - protein, 5 aa, synthetic construct
TGGGG

SEQ ID NO: 22 - protein, 5 aa, synthetic construct
SGGGG

SEQ ID NO: 23 - protein, 4 aa, synthetic construct
TGGG

SEQ ID NO: 24 - protein, 4 aa, synthetic construct
SGGG

SEQ ID NO: 25 - protein, 5 aa, synthetic construct
GGGGS

SEQ ID NO: 26-31 - skipped (6 entries)

SEQ ID NO: 32 - protein, 344 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 21 aa, Apis sp.
MKFLVNVALVFMVVYISYIYA

SEQ ID NO: 34 - protein, 22 aa, unidentified
MDAMKRGLCCVLLLCGAVFVSP

SEQ ID NO: 35 - protein, 20 aa, unidentified
MGAAAKLAFAVFLISCSSGA

SEQ ID NO: 36 - protein, 369 aa, synthetic construct
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 1114 bp, synthetic construct
  source 1..1114: mol_type = other DNA
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 38 - protein, 9 aa, synthetic construct
ILGRSETQE

SEQ ID NO: 39-40 - skipped (2 entries)

SEQ ID NO: 41 - protein, 343 aa, synthetic construct
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42-61 - skipped (20 entries)

SEQ ID NO: 62 - protein, 115 aa, Ovis aries
ILGRSETQECIFYNANWERDRTNRTGVESCYGDKDKRRHCFATWKNISGSIEIVKQGCWLDDINCYDRTDCIEKKDSPEVYFCCCEGNMCNERFSYFPEMEVTQPTSNPVTPKPP

SEQ ID NO: 63-64 - skipped (2 entries)

SEQ ID NO: 65 - protein, 115 aa, Gallus sp.
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKPP

SEQ ID NO: 66 - protein, 115 aa, Bos taurus; identical to SEQ ID NO: 62

SEQ ID NO: 67 - protein, 114 aa, Tyto alba
ILGRSETQECIYYNANWEKDKTNRSGIEPCYGDKDKRRHCFATWKNISGSIEIVKQGCWLDDINCYDRNDCIEKKDSPEVFFCCCEGNMCNERFFYFPEMEVTQPTSNPVTPKP

SEQ ID NO: 68 - protein, 114 aa, Myotis davidii
ILGRSETQECIFYNANWERDKTNRTGVELCYGDKDKRRHCFATWKNISGSIEIVKQGCWLDDINCYDRTDCIEKKDSPEVYFCCCEGNMCNERFSYFPEMEVTQPTSNPVTPKP

SEQ ID NO: 69-132 - skipped (64 entries)

SEQ ID NO: 133 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 135 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVLHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 136 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

(19) United States
(12) Patent Application Publication
(10) Pub. No.: US 2026/0055163 A1
(43) Pub. Date: Feb. 26, 2026

(54) FORMULATIONS COMPRISING ACTRIIA PROTEIN VARIANTS

(71) Applicant: MERCK SHARP & DOHME LLC, Rahway, NJ (US)

(72) Inventors: Jainik P. Panchal, Rahway, NJ (US); Jun Xu, Rahway, NJ (US); Nicholas A. Pierson, Rahway, NJ (US); Jorge Alex Pavon, Rahway, NJ (US); Jing Song, Rahway, NJ (US); Mark A. McCoy, Rahway, NJ (US); Morgan B. Giles, Rahway, NJ (US); Nisha Raman, Rahway, NJ (US)

(21) Appl. No.: 19/321,952

(22) Filed: Sep. 8, 2025

Related U.S. Application Data
(63) Continuation of application No. PCT/US2024/018854, filed on Mar. 7, 2024.
(60) Provisional application No. 63/451,195, filed on Mar. 9, 2023.
(60) Provisional application No. 63/626,394, filed on Jan. 29, 2024.

(51) Int. Cl. C07K 14/71; A61K 9/00; A61K 31/198; A61K 38/00; A61K 47/14; A61K 47/18; A61K 47/26

(52) U.S. Cl. CPC C07K 14/71; A61K 9/0019; A61K 31/198; A61K 47/14; A61K 47/183; A61K 47/26; A61K 38/00; C07K 2319/30

(57) ABSTRACT

In certain aspects, the present disclosure provides stable, liquid pharmaceutical formulations comprising a recombinant fusion protein comprising an extracellular domain (ECD) of human activin receptor type-IIA (ActRIIA) protein or variant thereof linked to a constant domain of an immunoglobulin, such as human IgG1 Fc domain and one or more pharmaceutical additives and/or excipients.

### CROSS-REFERENCE TO RELATED APPLICATIONS

This application is a continuation of International Patent Application No. PCT/US2024/018854, filed Mar. 7, 2024, which claims the benefit of priority from U.S. Provisional Application No. 63/451,195, filed Mar. 9, 2023 and from U.S. Provisional Application No. 63/626,394, filed Jan. 29, 2024, the contents of each of which are incorporated herein by reference.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Sep. 8, 2025, is named Merck_220307_Sequence_Listing.xml and is 51.4 KB bytes in size.

### FIELD

Described herein are stable, liquid pharmaceutical formulations comprising a recombinant fusion protein comprising an extracellular domain (ECD) of human activin receptor type-IIA (ActRIIA) protein or variant thereof linked to a constant domain of an immunoglobulin, such as human IgG1 Fc domain and one or more pharmaceutical additives and/or excipients.

### BACKGROUND

Pulmonary hypertension (PH) is a disease characterized by high blood pressure in lung vasculature, including pulmonary arteries, pulmonary veins, and pulmonary capillaries. In general, PH is defined as a mean pulmonary arterial (PA) pressure ≥25 mm Hg at rest or ≥30 mm Hg with exercise (Hill et al., Respiratory Care 54(7):958-68 (2009)). The main PH symptom is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches (Simonneau et al., JACC 54(1):S44-54 (2009)). Clinical classification of PH was first proposed in 1973, and a recent updated clinical classification was endorsed by the World Health Organization (WHO) in 2008. According to the updated PH clinical classification, there are five main groups of PH: (1) pulmonary arterial hypertension (PAH), characterized by a PA wedge pressure 15 mm Hg; (2) PH owing to a left heart disease (also known as pulmonary venous hypertension or congestive heart failure), (3) PH characterized by a PA wedge pressure >15 mm Hg; (4) PH owing to lung diseases and/or hypoxia; chronic thromboemboli PH; and (5) PH with unclear or multifactorial etiologies (Simonneau et al., JACC 54(1):S44-54 (2009); Hill et al., Respiratory Care 54(7):958-68 (2009)). PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of newborns (Simonneau et al., JACC 54(1):S44-54 (2009)). Diagnosis of various types of PH requires a series of tests.

In general, PH treatment depends on the cause or classification of the PH. Where PH is caused by a known medicine or medical condition, it is known as a secondary PH, and its treatment is usually directed at the underlying disease. Treatment of pulmonary venous hypertension generally involves optimizing left ventricular function by administering diuretics, beta blockers, and ACE inhibitors, or repairing or replacing a mitral valve or aortic valve. PAH therapies include pulmonary vasodilators, digoxin, diuretics, anticoagulants, and oxygen therapy. Pulmonary vasodilators target different pathways, including prostacyclin pathway (e.g., prostacyclins, including intravenous epoprostenol, subcutaneous or intravenous treprostinil, and inhaled iloprost), nitric oxide pathway (e.g., phosphodiesterase-5 inhibitors, including sildenafil and tadalafil), and endotheline-1 pathway (e.g., endothelin receptor antagonists, including oral bosentan and oral ambrisentan) (Humbert, M. Am. J. Respir. Crit. Care Med. 179:650-6 (2009); Hill et al., Respiratory Care 54(7):958-68 (2009)). However, current therapies provide no cure for PH, and they do not directly treat the underling vascular remodeling and muscularization of blood vessels observed in many PH patients.

Thus, it is an object of the present disclosure to provide stable liquid pharmaceutical formulations comprising an ActRIIa fusion protein and corresponding methods for treating, preventing, or reducing the progression rate and/or severity of PH, particular treating, preventing or reducing the progression rate and/or severity of one or more PH-associated complications.

### SUMMARY

Provided herein are stable, liquid pharmaceutical formulations comprising a recombinant fusion protein comprising an extracellular domain (ECD) of human activin receptor type-IIA (ActRIIa) or a variant thereof linked to a constant domain of an immunoglobulin, such as a human IgG1 Fc domain, and one or more pharmaceutical additives and/or excipients.

In some embodiments, the ActRIIa protein comprises an amino acid sequence that is at least 70% (e.g., at least 70%, 75%, 80%, 85%, 86%, 87%, 88%, 89%, 90%, 91%, 92%, 93%, 94%, 95%, 96%, 97%, 98%, 99%, or 100%) identical to an amino acid sequence that begins at any one of amino acids 21, 22, 23, 24, 25, 26, 27, 28, 29, or 30 of SEQ ID NO: 9 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 9. In some embodiments, the ActRIIa protein comprises an amino acid sequence that is at least 70% (e.g., at least 70%, 75%, 80%, 85%, 86%, 87%, 88%, 89%, 90%, 91%, 92%, 93%, 94%, 95%, 96%, 97%, 98%, 99%, or 100%) identical to the amino acid sequence of SEQ ID NO: 10. In some embodiments, the ActRIIa protein comprises an amino acid sequence that is at least 70% (e.g., at least 70%, 75%, 80%, 85%, 86%, 87%, 88%, 89%, 90%, 91%, 92%, 93%, 94%, 95%, 96%, 97%, 98%, 99%, or 100%) identical to the amino acid sequence of SEQ ID NO: 11.

In some embodiments, the ActRIIa protein is an ActRIIa fusion protein comprising an ActRIIa extracellular domain and one or more protein domains heterologous to ActRIIa. In some embodiments, the ActRIIa protein is a fusion protein comprising an Fc domain of an immunoglobulin. In some embodiments, the Fc domain of the immunoglobulin is an Fc domain of an IgG1 immunoglobulin. In some embodiments, the ActRIIa fusion protein further comprises a linker domain positioned between the ActRIIa protein domain and the one or more heterologous domains (e.g., an Fc immunoglobulin domain). In some embodiments, the linker domain is selected from the group consisting of: TGGG (SEQ ID NO: 23), TGGGG (SEQ ID NO: 21), SGGGG (SEQ ID NO: 22), GGGGS (SEQ ID NO: 25), GGG (SEQ ID NO: 19), GGGG (SEQ ID NO: 20), and SGGG (SEQ ID NO: 24). In some embodiments, the ActRIIa fusion protein comprises an amino acid sequence that is at least 70%, 75%, 80%, 85%, 86%, 87%, 88%, 89%, 90%, 91%, 92%, 93%, 94%, 95%, 96%, 97%, 98% or 99% identical to the amino acid sequence of SEQ ID NO: 32. In some embodiments, the ActRIIa fusion protein comprises the amino acid sequence of SEQ ID NO: 32. In some embodiments, the ActRIIa fusion protein consists of the amino acid sequence of SEQ ID NO: 32. In some embodiments, the ActRIIa fusion protein consists of the amino acid sequence of SEQ ID NO: 41. In some embodiments, the ActRIIa fusion protein consists of the amino acid sequence of SEQ ID NO: 32 or 41. In some embodiments, the ActRIIa fusion protein consists of a variant of the amino acid sequence set forth in SEQ ID NO: 32, wherein the sequence is lacking the C-terminal lysine residue of SEQ ID NO:32. In some embodiments, the ActRIIa fusion protein variant lacking the C-terminal lysine residue comprises or consists of the amino acid sequence of SEQ ID NO:41. In some embodiments, the ActRIIa fusion protein is part of a homodimer protein complex. In some embodiments, the ActRIIa fusion protein is glycosylated. In some embodiments, the ActRIIa fusion protein has a glycosylation pattern obtainable by expression in a Chinese hamster ovary cell.

In certain embodiments, the pharmaceutical formulations described herein, comprise a ActRIIA fusion protein, or a variant, a buffer, a surfactant, a stabilizing agent, and optionally one or more antioxidants. In certain embodiments, the pharmaceutical formulations described herein, comprise a ActRIIA fusion protein of SEQ ID NO: 32, or a variant of SEQ ID NO: 32 lacking the C-terminal lysine, a buffer, a surfactant, a stabilizing agent, and optionally one or more antioxidants.

In some embodiments, the pharmaceutical formulations provided herein comprise 10-100 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain. In other embodiments the pharmaceutical formulations comprise 1, 2, 3, 4, 5, 6, 7, 8, 9, 10, 20, 30, 40, 50, 60, 70, 80, 90 or 100 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain. In other embodiments the pharmaceutical formulations comprise 40-50 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain. In other embodiments the pharmaceutical formulations comprise 50 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain.

In certain embodiments, the pharmaceutical formulation provided herein comprises 10-100 mg/mL of a human ActRIIA fusion protein of SEQ ID NO: 32, or a variant of SEQ ID NO: 32 lacking the C-terminal lysine. In certain embodiments, the pharmaceutical formulation provided herein comprise 50-100 mg/mL of a human ActRIIA fusion protein of SEQ ID NO: 32, or a variant of SEQ ID NO: 32 lacking the C-terminal lysine. In certain embodiments, the pharmaceutical formulation provided herein comprises 40-50 mg/mL of a human ActRIIa fusion protein of SEQ ID NO: 32, or a variant of SEQ ID NO: 32 lacking the C-terminal lysine. In certain embodiments, the pharmaceutical formulation provided herein comprises 100 mg/mL of a human ActRIIa fusion protein of SEQ ID NO: 32, or a variant of SEQ ID NO: 32 lacking the C-terminal lysine. In certain embodiments, the pharmaceutical formulation provided herein comprises 50 mg/mL of a human ActRIIa fusion protein of SEQ ID NO: 32, or a variant of SEQ ID NO: 32 lacking the C-terminal lysine.

In certain embodiments, the pharmaceutical formulation provided herein comprises 10-100 mg/mL of a human ActRIIa fusion protein of SEQ ID NO: 32 and/or SEQ ID NO:41. In certain embodiments, the pharmaceutical formulation provided herein comprises 50-100 mg/mL of a human ActRIIa fusion protein of SEQ ID NO: 32 and/or SEQ ID NO:41. In certain embodiments, the pharmaceutical formulation provided herein comprises 40-50 mg/mL of a human ActRIIa fusion protein of SEQ ID NO: 32 and/or SEQ ID NO:41. In certain embodiments, the pharmaceutical formulation provided herein comprises 50 mg/mL of a human ActRIIa fusion protein of SEQ ID NO: 32 and/or SEQ ID NO:41. In certain embodiments, the pharmaceutical formulation provided herein comprises 100 mg/mL of a human ActRIIa fusion protein of SEQ ID NO: 32 and/or SEQ ID NO:41.

In certain embodiments, the pharmaceutical formulation provided herein comprises 10-100 mg/mL of a human ActRIIa fusion protein, a buffer, a surfactant, a stabilizing agent, and optionally one or more antioxidants, wherein the buffer is not histidine.

In certain embodiments, the pharmaceutical formulation provided herein comprises 10-100 mg/mL of a human ActRIIa fusion protein of SEQ ID NO: 32 and/or SEQ ID NO:41, a buffer, a surfactant, a stabilizing agent, and optionally one or more antioxidants, wherein the buffer is not histidine.

In certain embodiments, the pharmaceutical formulation provided herein comprises 10-100 mg/mL of a human ActRIIa fusion protein of SEQ ID NO: 32, a buffer, a surfactant, a stabilizing agent, and optionally one or more antioxidants, wherein the buffer is not histidine.

In certain embodiments, the pharmaceutical formulations provided herein comprise 50-100 mg/mL of a human ActRIIa fusion protein of SEQ ID NO: 32, or a variant of SEQ ID NO: 32 lacking the C-terminal lysine, a buffer, a surfactant, a stabilizing agent, and optionally one or more antioxidants, wherein the buffer is not histidine.

In some embodiments, the buffering agent comprises organic acids, succinate, phosphate, acetate, citrate, citric acid, Tris, HEPES, glutamate, amino acids, MES (2-(N-morpholino)ethanesulfonic acid), lactate or mixtures of amino acids. In some embodiments, the buffering agent comprises organic acids, succinate, phosphate, acetate, citrate, citric acid, Tris, HEPES, glutamate, amino acids, or mixtures of amino acids. In some embodiments, the buffering agent comprises tri-sodium citrate dihydrate. In some embodiments, the buffering agent comprises succinate, phosphate, acetate, citrate, lactate or glutamate. In some embodiments, the buffering agent comprises succinate, phosphate, acetate, citrate or glutamate. In some embodiments, the buffering agent comprises tri-sodium citrate dihydrate. In some embodiments, the buffering agent comprises citric acid monohydrate. In some embodiments, the buffering agent comprises citrate. In some embodiments, the buffering agent comprises tri-sodium citrate dihydrate and citric acid monohydrate. In certain embodiments, wherein the protein has a negative charge due to the presence of glycans, the buffer is not histidine.

In some embodiments, the buffering agent is selected to be physiologically compatible and to maintain a pH above 4. In some embodiments, the buffering agent is selected to be physiologically compatible and to maintain a pH between 5-7. In some embodiments, the buffering agent is selected to be physiologically compatible and to maintain a pH of 4.0, 4.1, 4.2, 4.3, 4.4, 4.5, 4.6, 4.7, 4.8, 4.9, 5.0, 5.1, 5.2, 5.3, 5.4, 5.5, 5.6 5.7, 5.8, 5.9 6.0, 6.1, 6.2, 6.3, 6.4, 6.5, 6.6 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments, the buffering agent is selected to be physiologically compatible and to maintain a pH of 5.5, 5.6 5.7, 5.8, 5.9 6.0, 6.1, 6.2, 6.3, 6.4, 6.5, 6.6 6.7, 6.8, 6.9, or 7.0. In some embodiments, the buffering agent is selected to be physiologically compatible and to maintain a pH of 5.8.

In certain embodiments, the pharmaceutical formulations described herein include a buffer, wherein the buffer is a phosphate buffer to maintain the pH of the pharmaceutical formulation between 5-7. In certain embodiments, the pharmaceutical formulations described herein include a buffer, wherein the buffer is a citrate buffer to maintain the pH of the pharmaceutical formulation pH between 4.5-7. In certain embodiments, the pharmaceutical formulations described herein include a buffer, wherein the buffer is an acetate buffer to maintain the pH of the pharmaceutical formulation pH between 4.5-6. In certain embodiments, the pharmaceutical formulations described herein include a buffer, wherein the buffer is a succinate buffer to maintain the pH of the pharmaceutical formulation pH between 4.5-5. In certain embodiments, the pharmaceutical formulations described herein include a buffer, wherein the buffer is a glutamate buffer to maintain the pH of the pharmaceutical formulation pH between 5-7.

In some embodiments, the buffering agent is present at a concentration of at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In some embodiments, the buffering agent is present at a concentration between 10-50 mM. In some embodiments, the buffering agent is present at a concentration of at least 10 mM.

In some embodiments, the buffering agent is 10 mM of a citrate buffer that maintains a pH of the pharmaceutical formulation at a pH from about 5.5 to about pH 6.5. In some embodiments, the buffering agent is 10 mM of a succinate buffer that maintains a pH of the pharmaceutical formulation at a pH from about 5.5 to about pH 6.5. In some embodiments, the buffering agent is 10 mM of a histidine buffer that maintains a pH of the pharmaceutical formulation at a pH from about 5.5 to about pH 6.0. In some embodiments, the buffering agent is 10 mM of a citrate buffer that maintains a pH of the pharmaceutical formulation at a pH of about 5.8. In some embodiments, the buffering agent is 10 mM of a succinate buffer that maintains a pH of the pharmaceutical formulation at a pH of about 5.8.

In some embodiments, the stabilizing agent is selected from the group consisting of: carboxymethylcellulose (CMC), dextrose, polyethylene glycol (PEG), albumin, kelptose, proline, sucrose, trehalose, mannose, maltose, lactose, glucose, raffinose, cellobiose, gentiobiose, isomaltose, arabinose, glucosamine, fructose, mannitol, sorbitol, poly-hydroxy compounds, polysaccharides, dextran, starch, hydroxyethyl starch, cyclodextrins, N-methyl pyrollidene, cellulose, and hyaluronic acid. In some embodiments, the stabilizing agent is selected from the group consisting of: sucrose, trehalose, mannose, maltose, lactose, glucose, raffinose, cellobiose, gentiobiose, isomaltose, arabinose, glucosamine, fructose, mannitol, sorbitol, poly-hydroxy compounds, polysaccharides, dextran, starch, hydroxyethyl starch, cyclodextrins, N-methyl pyrollidene, cellulose, and hyaluronic acid. In some embodiments, the stabilizing agent is sucrose.

In some embodiments, the stabilizing agent is present in the formulation at a concentration between 2-16% weight/volume. In some embodiments, the stabilizing agent is present at a concentration between 6-10% weight/volume. In some embodiments, the stabilizing agent is present at a concentration of at least 0.005% weight/volume, 0.01% weight/volume, 0.02% weight/volume, 0.03% weight/volume, 0.05% weight/volume, 0.06% weight/volume, 0.07% weight/volume, 0.08% weight/volume, 0.09% weight/volume, 0.10% weight/volume, 0.5% weight/volume, 0.7% weight/volume, 0.8% weight/volume, 0.9% weight/volume, 1.0% weight/volume, 1.2% weight/volume, 1.5% weight/volume, 1.7% weight/volume, 2% weight/volume, 3% weight/volume, 4% weight/volume, 5% weight/volume, 6% weight/volume, 7% weight/volume, 8% weight/volume, 9% weight/volume, 10% weight/volume, 11% weight/volume, 12% weight/volume, 13% weight/volume, 14% weight/volume, 15% weight/volume, 16% weight/volume, 17% weight/volume, 18% weight/volume, 19% weight/volume, or 20% weight/volume. In some embodiments, the stabilizing agent is present at a concentration of at least 8% weight/volume.

In some embodiments, the surfactant is selected from the group consisting of: sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt, benzalkonium chloride, benzethonium chloride, cetylpyridinium chloride monohydrate, hexadecyltrimethylammonium bromide, CHAPS, CHAPSO, SB3-10, SB3-12, digitonin, Triton X-100, Triton X-114, TWEEN-20, TWEEN-80, lauromacrogol 400, polyoxyl 40 stearate, polyoxyethylene hydrogenated castor oil 10, 40, 50 and 60, glycerol monostearate, polysorbate 20, polysorbate 40, polysorbate 60, polysorbate 65, polysorbate 80, polaxamer188 and soy lecithin. In certain embodiments, the surfactant is selected from the group consisting of polaxamer188, sodium dodecyl sulfate (SDS), N-dodecyl-β-D-maltoside (DDM), polysorbate 20 and triton X. In certain embodiments, the surfactant is selected from the group consisting of polysorbate 20, polysorbate 80, poloxamer 124, poloxamer 127, poloxamer 188, and poloxamer 407.

In some embodiments, the surfactant is polysorbate 80. In some embodiments, the surfactant is polysorbate 20. In some embodiments, the surfactant is polaxamer 188.

In some embodiments, the surfactant is present in the formulation at a concentration of 0.02-2.0 mg/mL. In some embodiments, the surfactant is present in the formulation at a concentration of 0.02 mg/mL, 0.03 mg/mL, 0.04 mg/mL, 0.05 mg/mL, 0.06 mg/mL, 0.07 mg/mL, 0.08 mg/mL, 0.09 mg/mL, 0.1 mg/mL, 0.2 mg/mL, 0.3 mg/mL, 0.4 mg/mL, 0.5 mg/mL, 0.6 mg/mL, 0.7 mg/mL, 0.8 mg/mL, 0.9 mg/mL, 1 mg/mL, 1.2 mg/mL, 1.3 mg/mL, 1.4 mg/mL, 1.5 mg/mL, 1.6 mg/mL, 1.7 mg/mL, 1.8 mg/mL, 1.9 mg/mL, and 2.0 mg/mL. In some embodiments, the surfactant is present in the formulation at a concentration of 0.05-0.3 mg/mL. In some embodiments, the surfactant is present at a concentration of at least 0.05, 0.06, 0.07, 0.08, 0.09, 0.1, 0.2, 0.3, 0.4, 0.5, 0.6, 0.7, 0.8 0.9, or 1.0 mg/mL. In some embodiments, the pharmaceutical formulation comprises a concentration of at least 0.2 mg/mL of a surfactant. In some embodiments, the pharmaceutical formulation comprises a concentration of at least 0.05, 0.06, 0.07, 0.08, 0.09, 0.1, 0.2, 0.3, 0.4 or 0.5 mg/mL of a surfactant. In some embodiments, the pharmaceutical formulation comprises a concentration of 0.05-0.5 mg/mL of a surfactant. In some embodiments, the pharmaceutical formulation comprises a concentration of 0.1-0.5 mg/mL of a surfactant.

In some embodiments, one or more of the pharmaceutical additives and/or excipients is an antioxidant. In some embodiments of the pharmaceutical formulations provided herein, the antioxidant concentration is between 0.001-50 mM. In some embodiments of the pharmaceutical formulations provided herein, the antioxidant concentration is between 7.5-50 mM. In some embodiments of the pharmaceutical formulations provided herein, the antioxidant concentration is between 5-20 mM. In some embodiments of the pharmaceutical formulations provided herein, the antioxidant concentration is at least 1, 2, 3, 4, 5, 6, 7, 8, 9, 10, 11, 12, 13, 14, 15, 16, 17, 18, 19, 20, 25, 30, 40, or 50 mM.

In one embodiment, the pharmaceutical formulation comprises an antioxidant selected from the group consisting of methionine (L or D form), tryptophan (L or D form), dimercaprol and pyridoxine. In one embodiment, the pharmaceutical formulation comprises an antioxidant selected from the group consisting of methionine (L or D form), tryptophan (L or D form) and pyridoxine. In certain embodiments, the formulation includes L-methionine.

In certain embodiments, the formulations described herein include 1-50 mM of an antioxidant. In certain embodiments, the formulations described herein include 5 mM, 10 mM or 50 mM of an antioxidant. In certain embodiments, the formulation includes 1-30 mM of an antioxidant. In certain embodiments, the formulation includes 1-20 mM of an antioxidant. In certain embodiments, the formulation includes 5-15 mM of an antioxidant. In certain embodiments, the formulation includes 5-10 mM of an antioxidant. In certain embodiments, the formulation includes 10 mM, or at least 10 mM of an antioxidant.

In certain embodiments, the formulation includes 1-50 mM of L-methionine. In certain embodiments, the formulation includes 1-30 mM of L-methionine. In certain embodiments, the formulation includes 1-20 mM of L-methionine. In certain embodiments, the formulation includes 5-15 mM of L-methionine. In certain embodiments, the formulation includes 5-10 mM of L-methionine. In certain embodiments, the formulation includes 10 mM, or at least 10 mM of L-methionine. In certain embodiments, the formulations described herein include 5 mM, 10 mM or 50 mM of L-methionine.

In some embodiments of the pharmaceutical formulations described herein, the pharmaceutical formulation optionally includes a chelating agent. In certain embodiments, the chelating agent is DTPA or EDTA. In certain embodiments, the chelating agent has a concentration of 1, 2, 3, 4, 5, 6, 7, 8, 9, 10, 15, 20, 25, 30, 35, 40, 45, 50, 55, 60, 65, 70, 75, 80, 85, 90, 95 or 100 μM. In one embodiment, the liquid formulation includes 1-100 μM, 1-30 μM, 1-20 μM, 10 μM-30 μM of DTPA or EDTA. In certain embodiments, the chelating agent has a concentration between 7.5-100 μM. In certain embodiments, the chelating agent has a concentration of 10 μM.

In some embodiments, the pharmaceutical formulations provided herein comprise 10-100 mg/mL of a human ActRIIa fusion protein of SEQ ID NO: 32, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM or DTPA or EDTA. In other embodiments, the pharmaceutical formulations provided herein comprise 50 mg/mL of a human ActRIIa fusion protein of SEQ ID NO: 32, 10 mM of citrate buffer, 8% weight/volume of sucrose, 0.2 mg/mL of polysorbate 80, and 20 mM of L-methionine. In some embodiments, the pharmaceutical formulations provided herein comprise 10-100 mg/mL of a human ActRIIa fusion protein of SEQ ID NO: 32, or a variant of SEQ ID NO: 32 lacking the C-terminal lysine, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM or DTPA or EDTA.

In other embodiments, the pharmaceutical formulations provided herein comprise 50 mg/mL of a human ActRIIa fusion protein of SEQ ID NO: 32, or a variant of SEQ ID NO: 32 lacking the C-terminal lysine, 10 mM of citrate buffer, 8% weight/volume of sucrose, 0.2 mg/mL of polysorbate 80, and 20 mM of L-methionine.

In some embodiments, the pharmaceutical formulations provided herein comprise 10-100 mg/mL of a human ActRIIa fusion protein of SEQ ID NO: 32 and/or SEQ ID NO: 41, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM or DTPA or EDTA. In other embodiments, the pharmaceutical formulations provided herein comprise 50 mg/mL of a human ActRIIa fusion protein of SEQ ID NO: 32 and/or SEQ ID NO: 41, 10 mM of citrate buffer, 8% weight/volume of sucrose, 0.2 mg/mL of polysorbate 80, and 20 mM of L-methionine.

In some embodiments, the human ActRIIa fusion protein consists of the amino acid sequence of SEQ ID NO: 41. In some embodiments, the lyophilized formulation comprises a human ActRIIa fusion protein comprising the amino acid sequence of SEQ ID NO: 32 and a human ActRIIa fusion protein comprising the amino acid sequence of SEQ ID NO: 41. In some embodiments, the pharmaceutical formulation comprises sotatercept.

In certain embodiments, the pharmaceutical formulations described herein contain a mixture of SEQ ID NO: 32 and a variant of SEQ ID NO:32 lacking the C-terminal lysine residue (SEQ ID NO: 41). In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41.

In certain embodiments, the pharmaceutical formulation described herein contain 100% of SEQ ID NO: 32 by weight. In certain embodiments, the pharmaceutical formulation described herein contain 100% of SEQ ID NO: 41 by weight.

The pharmaceutical formulations provided herein can be used for the treatment of pulmonary arterial hypertension in a subject in need thereof. In certain embodiments, the pharmaceutical formulations provided herein are liquid.

In certain embodiments, the pharmaceutical formulations provided herein are stable when stored between 2-8° C. In certain embodiments, the liquid pharmaceutical formulations are stored under refrigerated conditions (temp. range: typically about 2-8° C., but under certain circumstances, the aqueous formulation may exhibit stability at other temperatures including at about 25° C. and about 40° C. for periods of up to about 3, 6, 9 or 12 months). In certain embodiments, the pharmaceutical formulation is administered via autoinjector. In certain embodiments, the pharmaceutical formulation is administered via subcutaneous injection. In some embodiments, the pharmaceutical formulation is administered parenterally.

In certain embodiments, the formulations described herein are contained in an injection device. In certain embodiments, the formulations described herein are contained in an injection device, wherein the injection device is an autoinjector. In other embodiments, the formulations described herein are contained in a glass vial.

### BRIEF DESCRIPTION OF THE DRAWINGS

FIG. 1 shows a multiple sequence alignment of various vertebrate ActRIIA proteins and human ActRIIA (SEQ ID NOs: 61-68).

FIGS. 2A and 2B show the purification of ActRIIA-hFc expressed in CHO cells. The protein purifies as a single, well-defined peak as visualized by sizing column (top panel) and Coomassie stained SDS-PAGE (bottom panel) (left lane: molecular weight standards; right lane: ActRIIA-hFc). See Example 1.

FIGS. 3A and 3B shows ActRIIA-hFc bound to activin with a dissociation constant (K_{D}) of 5×10⁻² and bound to GDF11 with a K_{D} of 9.96×10⁻⁹. See Example 1.

FIG. 4 shows results of Stability pH Screening Study: Effect of pH and Buffer at t=0. See Example 3: pH and Buffer Feasibility Studies.

FIG. 5 shows results of Stability pH Screening Study: Effect of pH and Buffer at 2 weeks at 40° C. See Example 3: pH and Buffer Feasibility Studies.

FIG. 6 shows results of Stability pH Screening Study: Effect of pH and Buffer at 1 month at 40° C. See Example 3: pH and Buffer Feasibility Studies.

FIG. 7 shows results of Stability pH Screening Study: Effect of pH and Buffer at 2 months at 40° C. See Example 3: pH and Buffer Feasibility Studies.

FIG. 8 shows 1H NMR data for SEQ ID NO:32 with and without DTPA. See Example 3: Effect of Metal Chelator on SEQ ID NO: 32.

FIG. 9 shows diffusion NMR data for SEQ ID NO:32 with and without DTPA. See Example 3: Effect of Metal Chelator on SEQ ID NO: 32.

FIG. 10 shows results of colloidal stability studies for SEQ ID NO:32 and PS80 carried out by agitation stress. See Example 3: Colloidal Stability and Surfactant Screen.

FIG. 11 shows results of colloidal stability studies for SEQ ID NO:32 and PS80 carried out by agitation stress. See Example 3: Colloidal Stability and Surfactant Screen.

FIG. 12 shows results of colloidal stability studies for SEQ ID NO:32 and PS80 carried out by freeze-thaw cycling. See Example 3: Colloidal Stability and Surfactant Screen.

FIG. 13 shows results of colloidal stability studies for SEQ ID NO:32 and PS80 carried out by freeze-thaw cycling. See Example 3: Colloidal Stability and Surfactant Screen.

FIG. 14 shows SEQ ID NO: 32 formulation with different levels (0 mM, 10 mM, 20 mM, 30 mM) of L-methionine were exposed to light stress in a photostability chamber in presence of different levels of either EDTA as chelating agents (0 μM, 7.5 μM, 15 μM, 30 μM, 60 μM). See Example 3: Chelator/L-Methionine ranging study.

FIG. 15 shows SEQ ID NO: 32 formulation with different levels (0 mM, 10 mM, 20 mM, 30 mM) of L-methionine were exposed to light stress in a photostability chamber in presence of different levels of either DTPA as chelating agents (0 μM, 7.5 μM, 15 μM, 30 μM, 60 μM). See Example 3: Chelator/L-Methionine ranging study.

### DETAILED DESCRIPTION

### Definitions

The terms used in this specification generally have their ordinary meanings in the art, within the context of this disclosure and in the specific context where each term is used. Certain terms are discussed below or elsewhere in the specification, to provide additional guidance to the practitioner in describing the formulations and methods of the disclosure and how to make and use them. The scope or meaning of any use of a term will be apparent from the specific context in which the term is used.

“About” and “approximately” shall generally mean an acceptable degree of error for the quantity measured given the nature or precision of the measurements that can occur, for example, through typical measuring, handling and sampling procedures involved in the preparation, characterization and/or use of the substance or composition; through inadvertent error in these procedures; through differences in the manufacture, source, or purity of the ingredients employed to make or use the compositions or carry out the procedures; and the like. Typically, exemplary degrees of error are within 10%, and more preferably within 5% of a given value or range of values. In certain embodiments, “about” can mean a variation of 0.1%, 0.5%, 1%, 2%, 3%, 4%, 5% or 10%.

Alternatively, and particularly in biological systems, the terms “about” and “approximately” may mean values that are within an order of magnitude, preferably within 5-fold and more preferably within 2-fold of a given value. Numerical quantities given herein are approximate unless stated otherwise, meaning that the term “about” or “approximately” can be inferred when not expressly stated.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Numeric ranges disclosed herein are inclusive of the numbers defining the ranges. For example, when a range of pH values is recited, such as “a pH between pH 5.5 and 6.0,” the range is intended to be inclusive of the recited values. For example, pH between 5.0 and 7.0 includes pH 5.0 and pH 7.0 and values between 5.0 and 7.0. As used herein, a formulation comprising “citrate buffer at pH X” refers to a solution at pH X and comprising the citrate buffer, i.e. the pH is intended to refer to the pH of the solution.

A protein disclosed herein can comprise an amino acid sequence which is not naturally occurring. Such variants necessarily have less than 100% sequence identity or similarity with the starting molecule. In certain embodiments, the variant will have an amino acid sequence from about 75% to less than 100% amino acid sequence identity or similarity with the amino acid sequence of the starting (e.g., naturally-occurring or wild-type) protein, more preferably from about 80% to less than 100%, more preferably from about 85% to less than 100%, more preferably from about 90% to less than 100% (e.g., 91%, 92%, 93%, 94%, 95%, 96%, 97%, 98%, 99%) and most preferably from about 95% to less than 100%, e.g., over the length of the variant molecule.

A “stable” formulation is one in which the protein therein essentially retains its physical stability and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs. (1991) and Jones, A. Adv. Drug Delivery Rev. 10:29-90 (1993). Stability can be measured at a selected temperature for a selected time period.

A “stable” liquid formulation is a pharmaceutical formulation with no significant changes observed at a refrigerated temperature (2-8° C.) for at least 3 months, preferably 6 months, and more preferably 1 year, and even more preferably up through 2 years. Additionally, a “stable” liquid formulation includes one that exhibits desired features at temperatures including at 25° C. and 40° C. for periods including 1 month, 3 months, 6 months, 12 months, and/or 24 months. Typical acceptable criteria for stability are as follows. Typically, no more than about 10%, preferably about 5% of protein is degraded as measured by SEC-HPLC. The pharmaceutical formulation is colorless, or clear to slightly opalescent by visual analysis. The concentration, pH and osmolality of the formulation have no more than +/−10% change. Potency is typically within 50-150 of the reference. Typically, no more than about 10%, preferably about 5% of aggregation is present in the formulation. The term “buffer” encompasses those agents which maintain the solution pH of the formulations of the invention in an acceptable range, or, for lyophilized formulations of the invention, provide an acceptable solution pH prior to lyophilization. The terms “lyophilization,” “lyophilized,” and “freeze-dried” refer to a process by which the material to be dried is first frozen and then the ice or frozen solvent is removed by sublimation in a vacuum environment. An excipient may be included in pre-lyophilized formulations to enhance stability of the lyophilized product upon storage. The term “pharmaceutical formulation” refers to preparations which are in such form as to permit the active ingredients to be effective, and which contains no additional components which are toxic to the subjects to which the formulation would be administered. The term “formulation” and “pharmaceutical formulation” are used interchangeably throughout. “Pharmaceutically acceptable” refers to excipients (vehicles, additives) and compositions that can reasonably be administered to a subject to provide an effective dose of the active ingredient employed and that are “generally regarded as safe” e.g., that are physiologically tolerable and do not typically produce an allergic or similar untoward reaction, such as gastric upset and the like, when administered to a human. In another embodiment, this term refers to molecular entities and compositions approved by a regulatory agency of the federal or a state government or listed in the U.S. Pharmacopeia or another generally recognized pharmacopeia for use in animals, and more particularly in humans. A “reconstituted” formulation is one that has been prepared by dissolving a lyophilized protein formulation in a diluent such that the protein is dispersed in the reconstituted formulation. The reconstituted formulation is suitable for administration, e.g. parenteral administration), and may optionally be suitable for subcutaneous administration.

### Pharmaceutical Formulations

Provided herein are pharmaceutical formulations comprising a recombinant fusion protein comprising an extracellular domain (ECD) of human activin receptor type-IIA (ActRIIA) proteins or derivatives thereof linked to a constant domain of an immunoglobulin, such as human IgG1 Fc domain. In certain aspects, the disclosure relates to stable, liquid pharmaceutical formulations comprising an extracellular domain (ECD) of a human ActRIIA protein or derivatives thereof linked to a constant domain of an immunoglobulin, such as human IgG1 Fc domain or injection.

The liquid formulations of this invention minimize the formation of aggregates (high molecular weight species) and particulates, improve colloidal stability, minimize fragmentation (low molecular weight species), and ensure that the protein maintains its biological activity over time. In certain embodiments, the formulation is made by taking an ActRIIA which is, for example, in an aqueous pharmaceutical formulation and buffer exchanging it into the desired buffer as the last step of the purification process. The ActRIIA is then concentrated to a desired concentration. There is no lyophilization step in this embodiment. Additionally, excipients such as stabilizers and surfactants can be added to the ActRIIA formulation, which is diluted using the appropriate buffer to final protein concentration. The final formulation is filtered and filled into a final container (e.g. an autoinjector). Alternatively, the formulation may be stored in a vial, and delivered through an injection device or vessel.

The pharmaceutical formulations provided herein comprise ActRIIa fusion proteins. In certain embodiments, the disclosure provides a pharmaceutical formulation comprising an ActRIIa fusion protein wherein the protein is present at a concentration about 1 mg/mL, 2 mg/mL, 3 mg/mL, 4 mg/mL, 5 mg/mL, 6 mg/mL, 7 mg/mL, 8 mg/mL, 8 mg/mL, 10 mg/mL, 15 mg/mL, about 17.5 mg/mL, about 20 mg/mL, about 22.5 mg/mL, about 25 mg/mL, about 27.5 mg/mL, about 30 mg/mL, about 32.5 mg/mL, about 35 mg/mL, about 37.5 mg/mL, about 40 mg/mL, about 42.5 mg/mL, about 45 mg/mL, about 47.5 mg/mL, about 50 mg/mL, about 52.5 mg/mL, about 55 mg/mL, about 57.5 mg/mL, about 60 mg/mL, about 62.5 mg/mL, about 65 mg/mL, about 67.5 mg/mL, about 70 mg/mL, about 72.5 mg/mL, about 75 mg/mL, about 77.5 mg/mL, about 80 mg/mL, about 82.5 mg/mL, about 85 mg/mL, about 90 mg/mL, about 92.5 mg/mL, about 95 mg/mL, about 97.5 mg/mL or about 100 mg/mL.

In certain embodiments, the ActRIIa fusion protein has a concentration of approximately 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL of the ActRIIa fusion protein. In other embodiments, the ActRIIa fusion protein has a concentration of 45 mg/mL, 46 mg/mL, 47 mg/mL, 48 mg/mL, 49 mg/mL, 50 mg/mL, 51 mg/mL, 52 mg/mL, 53 mg/mL, 54 mg/mL, or 55 mg/mL of the ActRIIa fusion protein. In some embodiments, the ActRIIa fusion protein has a concentration of approximately 50 mg/mL.

In some embodiments, the pharmaceutical formulations provided herein comprise 10-100 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain. In other embodiments the pharmaceutical formulations comprise 1, 2, 3, 4, 5, 6, 7, 8, 910, 20, 30, 40, 50, 60, 70, 80, 90 or 100 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain. In other embodiments the pharmaceutical formulations comprise 40-50 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain. In other embodiments the pharmaceutical formulations comprise 50 mg/mL of a recombinant fusion protein comprising an extracellular domain (ECD) of human activin receptor type-IIA (ActRIIA) proteins or derivatives thereof linked to a constant domain of an immunoglobulin, such as human IgG1 Fc domain.

In some embodiments, the liquid pharmaceutical formulations of ActRIIa fusion protein provided herein comprise a ActRIIa fusion protein and one or more pharmaceutical additives and/or excipients. In certain embodiments the one or more pharmaceutical additives and/or excipients comprises a buffer, stabilizing agent, a surfactant, and optionally one or more antioxidants which are described in greater detail below. Buffering agents may be selected to maintain the pH of the formulation during processing. Surfactants may be selected based on their ability to serve as an emulsifier, wetter, solubilizer and/or dispersant.

A person having ordinary skill in the art would recognize that the concentrations of the excipients described herein share an interdependency within a particular formulation. By way of example, the concentration of a bulking agent is, in one aspect, lowered where, e.g., there is a high protein concentration. Excipients and other additives are added to impart or enhance manufacturability and/or final product quality, such as the stability and delivery of a drug product (e.g., protein). The formulations provided herein comprise suitable excipients that enhance stability, and safety.

### Buffering Agents

Typically, the stability of a pharmacologically active protein formulation is observed to be maximal in a narrow pH range. This pH range of optimal stability needs to be identified early during pre-formulation studies. Several approaches, such as accelerated stability studies and calorimetric screening studies, are useful in this endeavor (Remmele R. L. Jr., et al., Biochemistry, 38(16): 5241-7 (1999)). Once a formulation is finalized, the protein must be manufactured and maintained throughout its shelf-life. Hence, buffering agents are almost always employed to control pH in the formulation.

Several factors must be considered when choosing a buffering agent. First and foremost, the buffer species and its concentration must be defined based on its pKa and the desired formulation pH. Equally important is to ensure that the buffer is compatible with the protein and other formulation excipients, and does not catalyze any degradation reactions. A third important aspect to be considered is the sensation of stinging and irritation the buffer may induce upon administration. The potential for stinging and irritation is greater for drugs that are administered via the subcutaneous (SC) or intramuscular (IM) routes, where the drug solution remains at the site for a relatively longer period of time than when administered by the IV route where the formulation gets diluted rapidly into the blood upon administration. For formulations that are administered by direct IV infusion, the total amount of buffer (and any other formulation component) needs to be monitored.

In some embodiments, the buffering agent comprises organic acids, succinate, phosphate, acetate, citrate, citric acid, Tris, HEPES, glutamate, amino acids, MES (2-(N-morpholino)ethanesulfonic acid), lactate or mixtures of amino acids. In some embodiments, the buffering agent comprises organic acids, succinate, phosphate, acetate, citrate, citric acid, Tris, HEPES, glutamate, amino acids, or mixtures of amino acids. In some embodiments, the buffering agent comprises tri-sodium citrate dihydrate. In some embodiments, the buffering agent comprises succinate, phosphate, acetate, citrate, lactate or glutamate. In some embodiments, the buffering agent comprises succinate, phosphate, acetate, citrate or glutamate. In some embodiments, the buffering agent comprises tri-sodium citrate dihydrate. In some embodiments, the buffering agent comprises citric acid monohydrate. In some embodiments, the buffering agent comprises citrate. In one embodiment, the buffering agent comprises tri-sodium citrate dihydrate and citric acid monohydrate. In another embodiment, the buffering agents are tri-sodium citrate dihydrate and citric acid monohydrate. In certain embodiments, wherein the protein has a negative charge due to the presence of glycans, the buffer will not be histidine.

In one embodiment, the buffering agent present in the formulation is selected to be physiologically compatible and to maintain a desired pH of the pharmaceutical formulation. In another embodiment, the pH of the formulation is above 4. In another embodiment, the pH of the formulation is between pH 4.0 and pH 12.0. For example, in various embodiments the pH of the reconstituted formulation is 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, or 9.0. In some embodiments, the pH of the formulation is between pH 5 and pH 7. In some embodiments, the pH of the stable liquid formulation is 5.5, 5.6, 5.7, 5.8, 5.9 6.0, 6.1, 6.2, 6.3, 6.4, 6.5, 6.6 6.7, 6.8, 6.9 or 7.0. In one embodiment, the pH of the stable liquid formulation is 5.8.

In certain embodiments, the buffer is a phosphate buffer to maintain a pH of the pharmaceutical formulation between 5-7. In certain embodiment, the buffer is a citrate buffer to maintain a pH of the pharmaceutical formulation between 4.5-7. In certain embodiment, the buffer is an acetate buffer to maintain a pH of the pharmaceutical formulation between 4.5-6. In certain embodiment, the buffer is a succinate buffer to maintain a pH of the pharmaceutical formulation between 4.5-5. In certain embodiment, the buffer is a glutamate buffer to maintain a pH of the pharmaceutical formulation between 5-7.

The pH buffering compound may be present in any amount suitable to maintain the pH of the formulation at a predetermined level. When appropriately low levels of buffer are used, crystallization and pH shifts may be avoided. In one embodiment, the concentration of the buffering agent is between 0.1 mM and 500 mM (1 M). For example, it is contemplated that the buffering agent is at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In certain embodiments, the buffering agent is 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In certain embodiments, the buffering agent is 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19 or 20 mM. In certain embodiments, the concentration of the buffering agent is 10 mM.

In some embodiments, the buffering agent comprises tri-sodium citrate dihydrate. In some embodiments, the buffering agent comprises citric acid monohydrate. In some embodiments, the buffering agent comprises citrate. In some embodiments, the buffering agent comprises tri-sodium citrate dihydrate and citric acid monohydrate. In certain embodiments, the buffer will not be histidine. In some embodiments, the buffering agent is present at a concentration of at least 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 25, 30, 40, 50, 60, 70, 80, 90, 100, 200, or 500 mM. In some embodiments, the concentration of the buffering agent is at least 10 mM.

### Stabilizing Agents

In certain embodiments, the pharmaceutical formulations provided herein comprise stabilizing agents. These stabilizing agents can be classified on the basis of the mechanisms by which they stabilize proteins against various chemical and physical stresses. Some stabilizing agents are used to alleviate the effects of a specific stress or to regulate a particular susceptibility of a specific protein. Other stabilizing agents have more general effects on the physical and covalent stabilities of proteins. Given the teachings and guidance provided herein, those skilled in the art will know what amount or range of stabilizing agent can be included in any particular formulation to achieve a formulation of the disclosure that is likely to promote retention and stability of the ActRIIa fusion protein.

In some embodiments, a stabilizing agent (or a combination of stabilizing agents) is added to the formulation to prevent or reduce storage-induced aggregation and chemical degradation. A hazy or turbid solution upon reconstitution normally indicates that the protein has precipitated or at least aggregated. Stabilizing agents are capable of preventing aggregation, or chemical degradation (for example, autolysis, deamidation, oxidation, etc.). Some stabilizing agents are also capable of acting as anticoagulants upon administration of the formulation to a patient. In some embodiments, the stabilizing agent is selected from the group consisting of: CMC, dextrose, PEG, albumin, kelptose, proline, sucrose, trehalose, mannose, maltose, lactose, glucose, raffinose, cellobiose, gentiobiose, isomaltose, arabinose, glucosamine, fructose, mannitol, sorbitol, poly-hydroxy compounds, polysaccharides, dextran, starch, hydroxyethyl starch, cyclodextrins, N-methyl pyrollidene, cellulose, and hyaluronic acid. In certain embodiments, the pharmaceutical formulations provided herein include stabilizing agents including but not limited to, sucrose, trehalose, mannose, maltose, lactose, glucose, raffinose, cellobiose, gentiobiose, isomaltose, arabinose, glucosamine, fructose, mannitol, sorbitol, poly-hydroxy compounds, including polysaccharides such as dextran, starch, hydroxyethyl starch, cyclodextrins, N-methyl pyrollidene, cellulose and hyaluronic acid (Carpenter et al., Develop. Biol. Standard 74:225, (1991)). In one embodiment of the disclosure, sucrose is used as a stabilizing agent.

In certain embodiments the formulation comprises a stabilizing agent in a concentration of about 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, 20, 30, 40, 50, 60, 70, 80, 90, 100, 200, 500, 700, 900, or 1000 mM. Likewise, in certain embodiments of the disclosure, the stabilizing agent is incorporated in a concentration of about 0.005, 0.01, 0.02, 0.03, 0.05, 0.06, 0.07, 0.08, 0.09, 0.1, 0.5, 0.7, 0.8 0.9, 1.0, 1.2, 1.5, 1.7, 2, 3, 4, 5, 6, 7, 8, 9, 10, 11, 12, 13, 14, 15, 16, 17, 18, 19, or 20% weight/volume. In certain embodiments, the stabilizing agent has a concentration between 2-16% weight/volume. In certain embodiments, stabilizing agent has a concentration between 6-10% weight/volume. In certain embodiments, the stabilizing agent has a concentration of at least 8% weight/volume. In certain embodiments, the stabilizing agent is sucrose and has a concentration between 2-16% weight/volume. In certain embodiments, stabilizing agent is sucrose and has a concentration between 6-10% weight/volume. In certain embodiments, the stabilizing agent is sucrose and has a concentration of at least 8% weight/volume.

### Surfactants

In certain embodiments, the pharmaceutical formulations provided herein may additionally include surfactants. Surfactants are commonly used in protein formulations to prevent surface-induced degradation. Surfactants are amphipathic molecules with the capability of out-competing proteins for interfacial positions (and/or promote proper refolding of a structurally altered protein molecule). Hydrophobic portions of the surfactant molecules occupy interfacial positions (e.g., air/liquid), while hydrophilic portions of the molecules remain oriented towards the bulk solvent. At sufficient concentrations (typically around the detergent's critical micellar concentration), a surface layer of surfactant molecules serve to prevent protein molecules from adsorbing at the interface. Thereby, surface-induced degradation is minimized. Surfactants contemplated herein include, without limitation, fatty acid esters of sorbitan polyethoxylates, i.e., polysorbate 20 and polysorbate 80. The two differ only in the length of the aliphatic chain that imparts hydrophobic character to the molecules, C-12 and C-18, respectively. Accordingly, polysorbate-80 is more surface-active and has a lower critical micellar concentration than polysorbate-20.

Detergents can also affect the thermodynamic conformational stability of proteins. Non-ionic surfactants are generally useful in protein stabilization. Ionic surfactants (detergents) normally destabilize proteins. Here again, the effects of a given detergent excipient will be protein specific. For example, polysorbates have been shown to reduce the stability of some proteins and increase the stability of others. Detergent destabilization of proteins can be rationalized in terms of the hydrophobic tails of the detergent molecules that can engage in specific binding with partially or wholly unfolded protein states. These types of interactions could cause a shift in the conformational equilibrium towards the more expanded protein states (i.e. increasing the exposure of hydrophobic portions of the protein molecule in complement to binding polysorbate). Alternatively, if the protein native state exhibits some hydrophobic surfaces, detergent binding to the native state may stabilize that conformation. Another aspect of polysorbates is that they are inherently susceptible to oxidative degradation. Often, as raw materials, they contain sufficient quantities of peroxides to cause oxidation of protein residue side-chains, especially methionine. The potential for oxidative damage arising from the addition of stabilizer emphasizes the point that the lowest effective concentrations of excipients should be used in formulations. For surfactants, the effective concentration for a given protein will depend on the mechanism of stabilization.

Surfactants are also added in appropriate amounts to prevent surface related aggregation phenomenon (Chang, B, J. Pharm. Sci. 85:1325, (1996)). Thus, exemplary surfactants include, without limitation, anionic, cationic, nonionic, zwitterionic, and amphoteric surfactants including surfactants derived from naturally-occurring amino acids. Anionic surfactants include, but are not limited to, sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt. Cationic surfactants include, but are not limited to, benzalkonium chloride or benzethonium chloride, cetylpyridinium chloride monohydrate, and hexadecyltrimethylammonium bromide. Zwitterionic surfactants include, but are not limited to, CHAPS, CHAPSO, SB3-10, and SB3-12. Non-ionic surfactants include, but are not limited to, digitonin, Triton X-100, Triton X-114, TWEEN-20, and TWEEN-80. Surfactants also include, but are not limited to lauromacrogol 400, polyoxyl 40 stearate, polyoxyethylene hydrogenated castor oil 10, 40, 50 and 60, glycerol monostearate, polysorbate 40, polysorbate 60, polysorbate 65 and polysorbate 80, soy lecithin and other phospholipids such as dioleyl phosphatidyl choline (DOPC), dimyristoylphosphatidyl glycerol (DMPG), dimyristoylphosphatidyl choline (DMPC), and (dioleyl phosphatidyl glycerol) DOPG; sucrose fatty acid ester, methyl cellulose and carboxymethyl cellulose. In certain embodiments, the surfactant is selected from the group consisting of polysorbate 20, polysorbate 80, poloxamer 124, poloxamer 127, poloxamer 188, and poloxamer 407. In certain embodiments, the surfactant is polysorbate 80. In certain embodiments, the surfactant is polysorbate 20. In certain embodiments, the surfactant is poloxamer 188.

In some embodiments, the surfactant is selected from the group consisting of sodium lauryl sulfate, dioctyl sodium sulfosuccinate and dioctyl sodium sulfonate, chenodeoxycholic acid, N-lauroylsarcosine sodium salt, lithium dodecyl sulfate, 1-octanesulfonic acid sodium salt, sodium cholate hydrate, sodium deoxycholate, and glycodeoxycholic acid sodium salt, benzalkonium chloride, benzethonium chloride, cetylpyridinium chloride monohydrate, hexadecyltrimethylammonium bromide, CHAPS, CHAPSO, SB3-10, SB3-12, digitonin, Triton X-100, Triton X-114, TWEEN-20, TWEEN-80, lauromacrogol 400, polyoxyl 40 stearate, polyoxyethylene hydrogenated castor oil 10, 40, 50 and 60, glycerol monostearate, polysorbate 20, polysorbate 40, polysorbate 60, polysorbate 65, polysorbate 80, polaxamer188 and soy lecithin. In certain embodiments, the surfactant is selected from the group consisting of polaxamer188, sodium dodecyl sulfate (SDS), N-dodecyl-β-D-maltoside (DDM), polysorbate 20 and triton X. In certain embodiments, the surfactant is selected from the group consisting of polysorbate 20, polysorbate 80, poloxamer 124, poloxamer 127, poloxamer 188, and poloxamer 407.

Formulations comprising these surfactants, either individually or as a mixture in different ratios, are therefore further provided. In the present formulations, the surfactant is incorporated in a concentration of about 0.01 to about 0.5 mg/ml. In the present formulations, the surfactant is incorporated in a concentration of about 0.05 to about 0.5 mg/ml. In the present formulations, the surfactant is incorporated in a concentration of about 0.1 to about 0.5 mg/ml. In various embodiments of the pharmaceutical formulations provided herein, the surfactant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 mg/ml. In the present formulations, the surfactant is incorporated in a concentration of 0.2 mg/ml. Likewise, in certain embodiments of the disclosure, the surfactant is incorporated in a concentration of about 0.001, 0.002, 0.003, 0.004, 0.005, 0.01, 0.02, 0.03, 0.05, 0.06, 0.07, 0.08, 0.09, 0.1, 0.2, 0.3, 0.4, 0.5, 0.7, 0.8 0.9, or 1.0% weight/volume.

In some embodiments, the surfactant is polysorbate 80. In some embodiments, the surfactant is present in the formulation at a concentration 0.05-0.3 mg/mL. In some embodiments, the surfactant is present at a concentration 0.2 mg/mL. In some embodiments, the surfactant is present at a concentration of at least 0.001, 0.002, 0.003, 0.004, 0.005, 0.01, 0.02, 0.03, 0.05, 0.06, 0.07, 0.08, 0.09, 0.1, 0.2, 0.3, 0.4, 0.5, 0.6, 0.7, 0.8 0.9, or 1.0% weight/volume. In some embodiments, the surfactant is present at a concentration of at least 0.02% weight/volume.

### Antioxidants

In certain embodiments, the pharmaceutical formulations described herein include one or more antioxidants. Oxidation of protein residues arises from a number of different sources. Beyond the addition of specific antioxidants, the prevention of oxidative protein damage involves the careful control of a number of factors throughout the manufacturing process and storage of the product such as atmospheric oxygen, temperature, light exposure, and chemical contamination.

The disclosure therefore contemplates the use of the pharmaceutical antioxidants including, without limitation, reducing agents, oxygen/free-radical scavengers, or chelating agents. Antioxidants in therapeutic protein formulations are, in one aspect, water-soluble and remain active throughout the product shelf-life. Reducing agents and oxygen/free-radical scavengers work by ablating active oxygen species in solution. In some embodiments of the pharmaceutical formulations described herein, the antioxidant concentration is 0.005, 0.01, 0.02, 0.03, 0.05, 0.06, 0.07, 0.08, 0.09, 0.1, 0.2, 0.3, 0.4, 0.5, 0.6, 0.7, 0.8, 0.9 or 1.0 mg/mL.

In one embodiment, the pharmaceutical formulations described herein comprise methionine (L or D form), tryptophan (L or D form), dimercaprol, or pyridoxine. In one embodiment, the pharmaceutical formulations described herein comprise methionine (L or D form), tryptophan (L or D form) or pyridoxine. In one embodiment, the pharmaceutical formulations described herein comprise methionine (L or D form). In one embodiment, the pharmaceutical formulations described herein comprise L-methionine. In one embodiment, the pharmaceutical formulations described herein comprise D-methionine.

In certain embodiments the pharmaceutical formulations described herein can also include 1-50 mM of an antioxidant. In certain embodiments the pharmaceutical formulations described herein can also include 1-30 mM of an antioxidant. In one embodiment, the pharmaceutical formulations described herein may also include 1-20 mM of an antioxidant. The pharmaceutical formulations described herein may also include 5-15 mM of an antioxidant. The pharmaceutical formulations described herein may also include 5-10 mM of an antioxidant. In certain embodiments, the pharmaceutical formulations described herein include 5 mM, 10 mM, or 50 mM of an antioxidant. The pharmaceutical formulations described herein may also include 10 mM, or at least 10 mM of an antioxidant.

In certain embodiments the pharmaceutical formulations described herein can also include 1-50 mM L-methionine. In certain embodiments the pharmaceutical formulations described herein can also include 1-30 mM L-methionine. In one embodiment, the pharmaceutical formulations described herein may also include 1-20 mM L-methionine. The pharmaceutical formulations described herein may also include 5-15 mM L-methionine. The pharmaceutical formulations described herein may also include 5-10 mM L-methionine. In certain embodiments, the pharmaceutical formulations described herein include 5 mM, 10 mM, or 50 mM of L-methionine. The pharmaceutical formulations described herein may also include 10 mM, or at least 10 mM of L-methionine.

### Chelating Agents

In certain embodiments, the pharmaceutical formulations described herein can optionally include one or more chelating agents. In certain embodiments, the chelating agent is DTPA or EDTA. The pharmaceutical formulations described herein can include 1, 2, 3, 4, 5, 6, 7, 8, 9, 10, 15, 20, 25, 30, 35, 40, 45, 50, 55, 60, 65, 70, 75, 80, 85, 90, 95 or 100 μM of a chelating agent. In certain embodiments, the stable liquid formulation includes between 1-100 μM, 1-30 μM, 1-20 μM, 10 μM-30 μM of DTPA or EDTA. In certain embodiments, the stable liquid formulation includes between 7.5-100 μM of DTPA or EDTA.

In certain embodiments, the stable liquid formulation includes 10 μM of DTPA or EDTA.

### ActRIIA Protein Formulations

In some embodiments of the pharmaceutical formulations provided herein, comprise 10-100 mg/mL of a human ActRIIa fusion protein, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM or DTPA or EDTA. In other embodiments of the pharmaceutical formulations provided herein, comprise, 50 mg/mL of a human ActRIIa fusion protein of SEQ ID NO: 32, or a variant of SEQ ID NO:32 lacking the C-terminal lysine, 10 mM of citrate buffer, 8% weight/volume of sucrose, 0.2 mg/mL of polysorbate 80, and 20 mM of L-methionine. In other embodiments of the pharmaceutical formulations provided herein, comprise, 50 mg/mL of a human ActRIIa fusion protein of SEQ ID NO: 32, 10 mM of citrate buffer, 8% weight/volume of sucrose, 0.2 mg/mL of polysorbate 80, and 20 mM of L-methionine.

In some embodiments of the pharmaceutical formulations provided herein, comprise 10-100 mg/mL of a human ActRIIa fusion protein, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM or DTPA or EDTA. In other embodiments of the pharmaceutical formulations provided herein, comprise, 50 mg/mL of a human ActRIIa fusion protein of SEQ ID NO: 41, 10 mM of citrate buffer, 8% weight/volume of sucrose, 0.2 mg/mL of polysorbate 80, and 20 mM of L-methionine.

In some embodiments of the pharmaceutical formulations provided herein, comprise 10-100 mg/mL of a human ActRIIa fusion protein, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM or DTPA or EDTA. In other embodiments of the pharmaceutical formulations provided herein, comprise, 50 mg/mL of a human ActRIIa fusion protein of a mixture of SEQ ID NO: 32 and SEQ ID NO: 41, 10 mM of citrate buffer, 8% weight/volume of sucrose, 0.2 mg/mL of polysorbate 80, and 20 mM of L-methionine.

In certain embodiments the dose is administered parenterally. In some embodiments, the dose is administered via subcutaneous injection. In some embodiments, the dose is administered via intradermal injection. In some embodiments, the dose is administered via intramuscular injection. In some embodiments, the dose is administered via intravenous injection. In some embodiments, the dose is self-administered.

### Stability

In certain embodiments, the liquid pharmaceutical formulations are stored under refrigerated conditions (temp. range: typically about 2-8° C., but under certain circumstances, the aqueous formulation may exhibit stability at other temperatures including at about 25° C. and about 40° C. for periods of up to about 3, 6 9 or 12 months). In certain embodiments, the pharmaceutical formulations provided herein are stable when stored between 2-8° C. In certain embodiments, the pharmaceutical formulations provided herein are stable when stored between 2-8° C. for 1, 3, 6 or 12 months. In certain embodiments, the pharmaceutical formulations provided herein are stable when stored at 5° C. In certain embodiments, the pharmaceutical formulations provided herein are stable when stored between 2-8° C. for 1, 3, 6 or 12 months. In certain embodiments, the pharmaceutical formulations provided herein are stable when stored at 5° C. In certain embodiments, the pharmaceutical formulations provided herein are stable when stored between 25° C. for 1, 3, 6 or 12 months. In certain embodiments, the pharmaceutical formulations provided herein are stable when stored at 5° C. In certain embodiments, the pharmaceutical formulations provided herein are stable when stored between 40° C. for 1, 3, 6 or 12 months.

### Kits

The present disclosure provides a kit comprising a pharmaceutical formulation provided herein and an injection device. In certain embodiments, the pharmaceutical formulation comprises an ActRIIA protein or ActRIIa fusion protein (e.g., a protein that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 9, SEQ ID NO: 32 or SEQ ID NO: 41, or fragments, functional variants, or modified forms thereof. In certain embodiments, the protein binds to one or more ligands selected from the group consisting of activin A, activin B, and GDF11. In certain such embodiments, the protein further binds to one or more ligands selected from the group consisting of BMP10, GDF8, and BMP6. In certain embodiments, the protein binds to activin and/or GDF11.

In some embodiments, the pharmaceutical formulation comprises a protein that comprises, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to SEQ ID NO: 9, SEQ ID NO: 32 or SEQ ID NO: 41. In certain such embodiments, the protein comprises an amino acid sequence that is least 90%, 95%, or 99% identical to SEQ ID NO: 9, SEQ ID NO: 32 or SEQ ID NO: 41, wherein the protein binds to activin and/or GDF11. In certain embodiments, the protein comprises the amino acid sequence of SEQ ID NO: 9, SEQ ID NO: 32 or SEQ ID NO: 41. In other embodiments, the protein consists of the amino acid sequence of SEQ ID NO: 9, SEQ ID NO: 32 or SEQ ID NO: 41.

In some embodiments, the pharmaceutical formulation comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41. In certain embodiments, the protein consists essentially of the amino acid sequence of SEQ ID NO: 41. In other embodiments, the protein consists of the amino acid sequence of SEQ ID NO: 41.

In some embodiments, the pharmaceutical formulation comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 9. In certain embodiments, the protein consists essentially of the amino acid sequence of SEQ ID NO: 9. In other embodiments, the protein consists of the amino acid sequence of SEQ ID NO: 9.

In some embodiments, the pharmaceutical formulation comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In certain embodiments, the protein consists essentially of the amino acid sequence of SEQ ID NO: 32. In other embodiments, the protein consists of the amino acid sequence of SEQ ID NO: 32.

In certain embodiments of the foregoing, the protein comprises a fusion protein further comprising an Fc domain of an immunoglobulin. In certain such embodiments, the Fc domain of the immunoglobulin is an Fc domain of an IgG1 immunoglobulin. In other embodiments, the fusion protein further comprises a linker domain positioned between the protein domain and the Fc domain of the immunoglobulin. In certain embodiments, the linker domain is a polyglycine linker.

In certain embodiments, the ActRIIA fusion protein is part of a homodimer protein complex.

In certain embodiments, the ActRIIA protein is glycosylated.

The present disclosure provides a kit comprising a pharmaceutical formulation described herein and an injection device. In certain embodiments, the kit comprises a pharmaceutical formulation described herein and an injection device In some embodiments of the kits disclosed herein, the pharmaceutical formulation comprising the protein is pre-filled in one or more containers, such as an autoinjector.

In certain embodiments, the pH range for the pharmaceutical formulation comprising a protein is from 5 to 7. In some embodiments, the pharmaceutical formulation comprising a protein further comprises a buffering agent. In some embodiments, the buffering agent is added in an amount of at least 10 mM. In some embodiments, the buffering agent is added in an amount in the range of between about 10 mM to about 200 mM. In some embodiments, the buffering agent comprises citrate.

In some embodiments, the pharmaceutical formulation comprising a protein further comprises a surfactant. In some embodiments, the surfactant comprises a polysorbate. In some embodiments, the surfactant comprises polysorbate 80 or polysorbate 20.

In some embodiments, the pharmaceutical formulation comprising a protein further comprises a sugar, such as disaccharides (e.g., sucrose). In some embodiments, the stable liquid pharmaceutical formulation comprising a protein comprises sucrose, trehalose, mannitol, polyvinylpyrrolidone (PVP), dextrose, and/or glycine. In some embodiments, the pharmaceutical formulation comprising a protein comprises sucrose. In some embodiments, the pharmaceutical formulation comprises a protein and a sugar in a weight ratio of at least 1:1 protein to sugar. In some embodiments, the pharmaceutical formulation comprises a protein and a sugar in a weight ratio from 1:1 to 1:10 protein to sugar. In some embodiments, the pharmaceutical formulation comprises a protein and a sugar in a weight ratio of 1:1, 1:2, 1:3, 1:4, 1:5, 1:6, 1:7, 1:8, 1:9, or 1:10 protein to sugar. In some embodiments, the pharmaceutical formulation comprises a protein and a sugar in a weight ratio of 1:6 protein to sugar. In certain embodiments of the foregoing, the pharmaceutical formulation comprises a protein and a sugar in an amount sufficient to stabilize the protein.

In certain embodiments of the kits disclosed herein, the injection device comprises a syringe. In certain such embodiments, the syringe is pre-filled with a stable liquid formulation.

In certain embodiments of the kits disclosed herein, the kit further comprises an injectable device for use in administering the sterile injectable solution parenterally. In some embodiments, the sterile injectable solution is administered via subcutaneous injection. In some embodiments, the sterile injectable solution is administered via intradermal injection. In some embodiments, the sterile injectable solution is administered via intramuscular injection. In some embodiments, the sterile injectable solution is administered via intravenous injection.

In certain embodiments of the kits disclosed herein, the kit further comprises an autoinjector for use in administering the sterile injectable solution. In some embodiments, the sterile injectable solution is self-administered. In some embodiments, the sterile injectable solution comprises a therapeutically effective dose. In some embodiments, the therapeutically effective dose comprises a weight based dose.

### ActRIIA Proteins

In certain embodiments, the present disclosure relates to ActRIIA proteins. As used herein, the term “ActRIIA” refers to a family of activin receptor type IIA (ActRIIA) proteins from any species and variants derived from such ActRIIA proteins by mutagenesis or other modification. Reference to ActRIIA herein is understood to be a reference to any one of the currently identified forms. Members of the ActRIIA family are generally transmembrane proteins, composed of a ligand-binding extracellular domain comprising a cysteine-rich region, a transmembrane domain, and a cytoplasmic domain with predicted serine/threonine kinase activity.

The term “ActRIIA protein” includes proteins comprising any naturally occurring protein of an ActRIIA family member as well as any variants thereof (including mutants, fragments, fusions, and peptidomimetic forms) that retain a useful activity. Examples of such variant ActRIIA proteins are provided throughout the present disclosure as well as in International Patent Application Publication Nos. WO 2006/012627 and WO 2007/062188, which are incorporated herein by reference in their entirety. Numbering of amino acids for all ActRIIA-related proteins described herein is based on the numbering of the human ActRIIA precursor protein sequence provided below (SEQ ID NO: 9), unless specifically designated otherwise.

The canonical human ActRIIA precursor protein sequence is as follows:

```
(SEQ ID NO: 9)
    1 MGAAAKLAFA VFLISCSSGA ILGRSETQEC LFFNANWEKD RTNQTGVEPC
  51 YGDKDKRRHC FATWKNISGS IEIVKQGCWL DDINCYDRTD CVEKKDSPEV
101 YFCCCEGNMC NEKFSYFPEM EVTQPTSNPV TPKPPYYNIL LYSLVPLMLI
151 AGIVICAFWV YRHHKMAYPP VLVPTQDPGP PPPSPLLGLK PLQLLEVKAR
201 GREGCVWKAQ LLNEYVAVKI FPIQDKQSWQ NEYEVYSLPG MKHENILQFI
251 GAEKRGTSVD VDLWLITAFH EKGSLSDELK ANVVSWNELC HIAETMARGL
301 AYLHEDIPGL KDGHKPAISH RDIKSKNVLL KNNLTACIAD FGLALKFEAG
351 KSAGDTHGQV GTRRYMAPEV LEGAINFQRD AFLRIDMYAM GLVLWELASR
401 CTAADGPVDE YMLPFEEEIG QHPSLEDMQE VVVHKKKRPV LRDYWQKHAG
451 MAMLCETIEE CWDHDAEARL SAGCVGERIT QMQRLTNIIT TEDIVTVVTM
501 VINVDFPPKE SSL
```

The signal peptide is indicated by a single underline; the extracellular domain is indicated in bold font; and the potential, endogenous N-linked glycosylation sites are indicated by a double underline.

The processed (mature) extracellular human ActRIIA protein sequence is as follows:

(SEQ ID NO: 10)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by single underline. The sequence with the “tail” deleted (a Δ15 sequence) is as follows:

(SEQ ID NO: 11)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM

The nucleic acid sequence encoding human ActRII precursor protein is shown below (SEQ ID NO: 12), as follows nucleotides 159-1700 of Genbank Reference Sequence NM_001616.4. The signal sequence is underlined.

```
(SEQ ID NO: 12)
      1 ATGGGAGCTG CTGCAAAGTT GGCGTTTGCC GTCTTTCTTA TCTCCTGTTC
    51 TTCAGGTGCT ATACTTGGTA GATCAGAAAC TCAGGAGTGT CTTTTCTTTA
  101 ATGCTAATTG GGAAAAAGAC AGAACCAATC AAACTGGTGT TGAACCGTGT
  151 TATGGTGACA AAGATAAACG GCGGCATTGT TTTGCTACCT GGAAGAATAT
  201 TTCTGGTTCC ATTGAAATAG TGAAACAAGG TTGTTGGCTG GATGATATCA
  251 ACTGCTATGA CAGGACTGAT TGTGTAGAAA AAAAAGACAG CCCTGAAGTA
  301 TATTTTTGTT GCTGTGAGGG CAATATGTGT AATGAAAAGT TTTCTTATTT
  351 TCCGGAGATG GAAGTCACAC AGCCCACTTC AAATCCAGTT ACACCTAAGC
  401 CACCCTATTA CAACATCCTG CTCTATTCCT TGGTGCCACT TATGTTAATT
  451 GCGGGGATTG TCATTTGTGC ATTTTGGGTG TACAGGCATC ACAAGATGGC
  501 CTACCCTCCT GTACTTGTTC CAACTCAAGA CCCAGGACCA CCCCCACCTT
  551 CTCCATTACT AGGTTTGAAA CCACTGCAGT TATTAGAAGT GAAAGCAAGG
  601 GGAAGATTTG GTTGTGTCTG GAAAGCCCAG TTGCTTAACG AATATGTGGC
  651 TGTCAAAATA TTTCCAATAC AGGACAAACA GTCATGGCAA AATGAATACG
  701 AAGTCTACAG TTTGCCTGGA ATGAAGCATG AGAACATATT ACAGTTCATT
  751 GGTGCAGAAA AACGAGGCAC CAGTGTTGAT GTGGATCTTT GGCTGATCAC
  801 AGCATTTCAT GAAAAGGGTT CACTATCAGA CTTTCTTAAG GCTAATGTGG
  851 TCTCTTGGAA TGAACTGTGT CATATTGCAG AAACCATGGC TAGAGGATTG
  901 GCATATTTAC ATGAGGATAT ACCTGGCCTA AAAGATGGCC ACAAACCTGC
  951 CATATCTCAC AGGGACATCA AAAGTAAAAA TGTGCTGTTG AAAAACAACC
1001 TGACAGCTTG CATTGCTGAC TTTGGGTTGG CCTTAAAATT TGAGGCTGGC
1051 AAGTCTGCAG GCGATACCCA TGGACAGGTT GGTACCCGGA GGTACATGGC
1101 TCCAGAGGTA TTAGAGGGTG CTATAAACTT CCAAAGGGAT GCATTTTTGA
1151 GGATAGATAT GTATGCCATG GGATTAGTCC TATGGGAACT GGCTTCTCGC
1201 TGTACTGCTG CAGATGGACC TGTAGATGAA TACATGTTGC CATTTGAGGA
1251 GGAAATTGGC CAGCATCCAT CTCTTGAAGA CATGCAGGAA GTTGTTGTGC
1301 ATAAAAAAAA GAGGCCTGTT TTAAGAGATT ATTGGCAGAA ACATGCTGGA
1351 ATGGCAATGC TCTGTGAAAC CATTGAAGAA TGTTGGGATC ACGACGCAGA
1401 AGCCAGGTTA TCAGCTGGAT GTGTAGGTGA AAGAATTACC CAGATGCAGA
1451 GACTAACAAA TATTATTACC ACAGAGGACA TTGTAACAGT GGTCACAATG
1501 GTGACAAATG TTGACTTTCC TCCCAAAGAA TCTAGTCTA
```

The nucleic acid sequence encoding processed soluble (extracellular) human ActRIIA protein is as follows:

```
(SEQ ID NO: 13)
    1 ATACTTGGTA GATCAGAAAC TCAGGAGTGT CTTTTCTTTA ATGCTAATTG
  51 GGAAAAAGAC AGAACCAATC AAACTGGTGT TGAACCGTGT TATGGTGACA
101 AAGATAAACG GCGGCATTGT TTTGCTACCT GGAAGAATAT TTCTGGTTCC
151 ATTGAAATAG TGAAACAAGG TTGTTGGCTG GATGATATCA ACTGCTATGA
201 CAGGACTGAT TGTGTAGAAA AAAAAGACAG CCCTGAAGTA TATTTTTGTT
251 GCTGTGAGGG CAATATGTGT AATGAAAAGT TTTCTTATTT TCCGGAGATG
301 GAAGTCACAC AGCCCACTTC AAATCCAGTT ACACCTAAGC CACCC
```

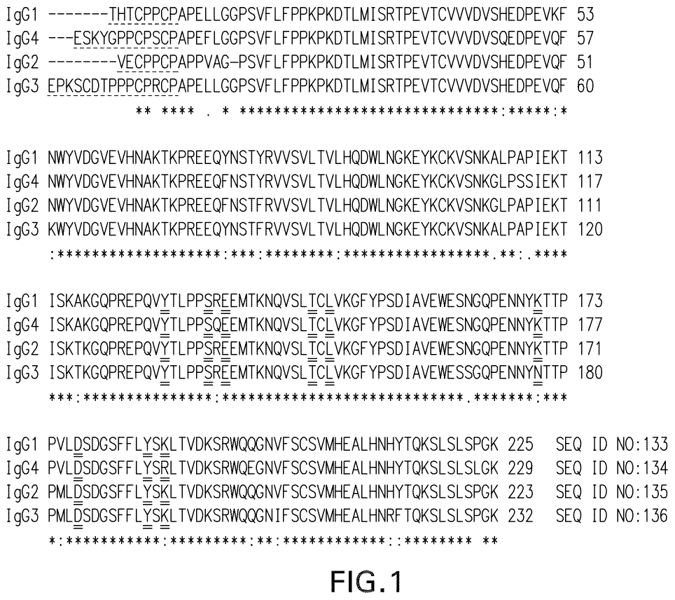

ActRIIA is well-conserved among vertebrates, with large stretches of the extracellular domain completely conserved. For example, FIG. 1 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRIIA-ligand binding activities as well as to predict amino acid positions that are likely to be tolerant to substitution without significantly altering normal ActRIIA-ligand binding activities. Therefore, an active, human ActRIIA variant protein useful in accordance with the presently disclosed methods may include one or more amino acids at corresponding positions from the sequence of another vertebrate ActRIIA, or may include a residue that is similar to that in the human or other vertebrate sequences.

Without meaning to be limiting, the following examples illustrate this approach to defining an active ActRIIA variant. As illustrated in FIG. 1, F13 in the human extracellular domain is Y in Ovis aries (SEQ ID NO: 62), Gallus (SEQ ID NO: 65), Bos Taurus (SEQ ID NO: 66), Tyto alba (SEQ ID NO: 67), and Myotis davidii (SEQ ID NO: 68) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding (Attisano et al. (1992) Cell 68(1):97-108; Greenwald et al. (1999) Nature Structural Biology 6(1): 18-22; Allendorph et al. (2006) PNAS 103(20: 7643-7648; Thompson et al. (2003) The EMBO Journal 22(7): 1555-1566; as well as U.S. Pat. Nos. 7,709,605, 7,612,041, and 7,842,663). In addition to the teachings herein, these references provide amply guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor (Greenwald et al. (1999) Nat Struct Biol 6:18-22; and Hinck (2012) FEBS Lett 586:1860-1870). Accordingly, the core ligand-binding domains of human ActRIIA, as demarcated by the outermost of these conserved cysteines, corresponds to positions 30-110 of SEQ ID NO: 9 (ActRIIA precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRIIA extracellular domains truncations include SEQ ID NOs: 10 and 11.

Accordingly, a general formula for an active portion (e.g., ligand binding) of ActRIIA is a protein that comprises, consists essentially of, or consists of amino acids 30-110 of SEQ ID NO: 9. Therefore ActRIIA proteins may, for example, comprise, consists essentially of, or consists of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to any one of amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 9. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 9, and end at a position selected from 111-135 (e.g., ending at any one of amino acids 111, 112, 113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 9. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 9. Thus, in some embodiments, an ActRIIA protein may comprise, consists essentially of, or consist of a protein that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9. Optionally, ActRIIA proteins comprise a protein that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 9, and comprising no more than 1, 2, 5, 10 or 15 conservative amino acid changes in the ligand-binding pocket.

In certain embodiments, the disclosure relates to GDF/BMP antagonists (inhibitors) that comprise an ActRIIA protein, which includes fragments, functional variants, and modified forms thereof as well as uses thereof (e.g., increasing an immune response in a patient in need thereof and treating cancer). Preferably, ActRIIA proteins are soluble (e.g., an extracellular domain of ActRIIA). In some embodiments, ActRIIA proteins inhibit (e.g., Smad signaling) of one or more GDF/BMP ligands (e.g., GDF11, GDF8, activin (activin A, activin B, activin AB, activin C, activin E) BMP6, GDF3, BMP15, and/or BMP10). In some embodiments, ActRIIA proteins bind to one or more GDF/BMP ligands (e.g., GDF11, GDF8, activin (activin A, activin B, activin AB, activin C, activin E) BMP6, GDF3, BMP15, and/or BMP10). In some embodiments, ActRIIA protein of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRIIA beginning at a residue corresponding to amino acids 21-30 (e.g., beginning at any one of amino acids 21, 22, 23, 24, 25, 26, 27, 28, 29, or 30) of SEQ ID NO: 9 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134 or 135) of SEQ ID NO: 9. In some embodiments, ActRIIA proteins comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 9. In certain embodiments, ActRIIA proteins comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 9. In some embodiments, ActRIIA proteins or ActRIIA fusion protein comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 9, 10, 11, 32, 36, 39 and 41.

In some embodiments, the ActRIIa fusion protein comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 32. In some embodiments, the ActRIIa fusion protein comprises the amino acid sequence of SEQ ID NO: 32. In some embodiments, the ActRIIa fusion protein consists of the amino acid sequence of SEQ ID NO: 32. In some embodiments, the ActRIIa fusion protein is part of a homodimer protein complex. In some embodiments, the ActRIIa fusion protein is glycosylated. In some embodiments, the ActRIIa fusion protein has a glycosylation pattern obtainable by expression in a Chinese hamster ovary cell.

In some alternative embodiments, the ActRII protein (e.g., SEQ ID NO: 32) lacks the C-terminal lysine. In some embodiments, the ActRII protein lacking the C-terminal lysine is SEQ ID NO: 41. In some embodiments, the formulation comprises a mixture of SEQ ID NO:32 and SEQ ID NO:41.

In certain embodiments, the pharmaceutical formulations described herein contain a mixture of SEQ ID NO: 32 and a variant of SEQ ID NO:32 lacking the C-terminal lysine residue (SEQ ID NO: 41). In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:32.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12, 11%, 10%, 9%, 8%, 7%, 6%, 5%, 4%, 3%, 2% or 1% by weight of SEQ ID NO:32.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11, 1%, 9%, 8%, 7%, 6%, 5%, 4%, 3%, 2% or 1% by weight of SEQ ID NO:41.

In certain embodiments, the pharmaceutical formulation described herein contain a mixture of SEQ ID NO: 32 and a variant of SEQ ID NO:32 lacking the C-terminal lysine residue (SEQ ID NO: 41) wherein the mixture contains about 99%, 98%, 97%, 96%, 95%, 94%, 93%, 92%, 91%, 90%, 89%, 88%, 87%, 86%, 85%, 84%, 83%, 82%, 81%, 80%, 79%, 78%, 77%, 76%, 75%, 74%, 73%, 72%, 71%, 70%, 69%, 68%, 67%, 66%, 65%, 64%, 63%, 62%, 61%, 60%, 59%, 58%, 57%, 56%, 55%, 54%, 53%, 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2% or 1% by weight of SEQ ID NO:41.

In certain embodiments, the pharmaceutical formulation described herein contain 100% of SEQ ID NO: 32 by weight. In certain embodiments, the pharmaceutical formulation described herein contain 100% of SEQ ID NO: 41 by weight.

In certain aspects, the present disclosure relates to GDF trap proteins (also referred to as “GDF traps”). In some embodiments, GDF traps of the present disclosure are variant ActRII proteins (e.g., ActRIIA) that comprise one or more mutations (e.g., amino acid additions, deletions, substitutions, and combinations thereof) in the extracellular domain (also referred to as the ligand-binding domain) of an ActRIIA protein (e.g., a “wild-type” or unmodified ActRIIA protein) such that the variant ActRIIA protein has one or more altered ligand-binding activities than the corresponding wild-type ActRIIA protein. In some embodiments, GDF trap proteins of the present disclosure retain at least one similar activity as a corresponding wild-type ActRIIA protein. For example, preferable GDF traps bind to and inhibit (e.g. antagonize) the function of GDF11 and/or GDF8. In some embodiments, GDF traps of the present disclosure further bind to and inhibit one or more of ligand of the GDF/BMP. Accordingly, the present disclosure provides GDF trap proteins that have an altered binding specificity for one or more ActRII ligands.

To illustrate, one or more mutations may be selected that increase the selectivity of the altered ligand-binding domain for GDF11 and/or GDF8 over one or more ActRIIA-binding ligands such as activins, particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

### Methods of Use

In certain aspects, the disclosure provides for a method of treating pulmonary arterial hypertension (PAH), comprising administering a pharmaceutical formulation described herein to a patient in need thereof.

In certain aspects, the disclosure provides a pharmaceutical formulation for the treatment of PAH in a subject in need thereof comprising administering a lyophilized human ActRIIA protein linked to a constant domain of an immunoglobulin, wherein the dosing regimen comprises: 1) administering an initial dose of 0.3 mg/kg; 2) administering a subsequent dose of 0.7 mg/kg three weeks after the initial dose; and 3) monitoring a subject's response; and 3) modifying the subsequent dose; and wherein the subject is administered the subsequent dose every three weeks. In some embodiments, the subsequent dose is modified based on the subject's response.

In certain aspects, the disclosure provides for a method of treating pulmonary arterial hypertension (PAH), comprising administering a pharmaceutical formulation described herein to a patient in need thereof, wherein administration of the pharmaceutical formulation results in a change in one or more of the following hemodynamic or functional parameters: a reduction in pulmonary vascular resistance (PVR); an increase in 6-minute walk distance (6MWD); a decrease of the N-terminal pro B-type natriuretic peptide (NT-proBNP) levels; the prevention or reduction of pulmonary hypertension Functional Class progression as recognized by the World Health Organization (WHO); the promotion or increasing of pulmonary hypertension Functional Class regression as recognized by the WHO; an improvement in right ventricular function; an improvement in pulmonary artery pressure; and/or an improvement in mean right atrial pressure.

In certain aspects, the disclosure provides a method of treating pulmonary arterial hypertension (PAH), comprising administering a pharmaceutical formulation described herein to a patient in need thereof, wherein administration of the pharmaceutical formulation results in increased exercise capacity, provide clinical improvement, improve WHO functional class (FC), and delay disease progression, including to reduce the risk of death and hospitalization for PAH.

In certain aspects, the disclosure provides for a method of treating, preventing, or reducing the progression rate and/or severity of one or more complications of pulmonary arterial hypertension, comprising administering to a patient in need thereof a pharmaceutical formulation described herein, wherein administration of said protein results in a change in one or more of the following hemodynamic or functional parameters: a reduction in pulmonary vascular resistance (PVR); an increase in 6-minute walk distance (6MWD); a decrease of the N-terminal pro B-type natriuretic peptide (NT-proBNP) levels; the prevention or reduction in pulmonary hypertension Functional Class progression as recognized by the World Health Organization (WHO); the promotion or increase of pulmonary hypertension Functional Class regression as recognized by the WHO; an improvement in right ventricular function; an improvement in pulmonary artery pressure; and/or an improvement in mean right atrial pressure. In some embodiments, the one or more complications of pulmonary arterial hypertension is selected from the group consisting of: smooth muscle and/or endothelial cell proliferation in the pulmonary artery, angiogenesis in the pulmonary artery, dyspnea, chest pain, pulmonary vascular remodeling, right ventricular hypertrophy, and pulmonary fibrosis.

In some embodiments, administration of the pharmaceutical formulations described herein reduce the PVR in the patient. In some embodiments, administration of the pharmaceutical formulations described herein reduce the PVR in the patient by at least 10% (e.g., 10%, 15%, 20%, 25%, 30%, 35%, 40%, 45%, or at least 50%). In some embodiments, administration of the pharmaceutical formulations described herein reduce the patient's PVR by at least 20%. In some embodiments, the reduction in PVR is a result of decreased mean pulmonary artery pressure. In some embodiments, administration of the pharmaceutical formulations described herein increase the patient's 6-minute walk distance. In some embodiments, administration of the pharmaceutical formulations described herein increase the patient's 6-minute walk distance by at least 10 meters (e.g., at least 10, 20, 30, 40, 50, 60, 70, 80, 90, 100, 125, 150, 175, 200, 250, 300, or more than 400 meters). In some embodiments, administration of the pharmaceutical formulations described herein increase the patient's 6-minute walk distance by at least 30 meters. In some embodiments, the administration of the pharmaceutical formulations described herein decrease NT-proBNP levels in the patient. In some embodiments, administration of the pharmaceutical formulations described herein decrease NT-proBNP levels in the patient by at least 10% (e.g., 10%, 15%, 20%, 25%, 30%, 35%, 40%, 45%, 50%, 55%, 60%, 65%, 70%, 75%, or at least 80%). In some embodiments, administration of the pharmaceutical formulations described herein decrease NT-proBNP levels in the patient by at least 30%. In some embodiments, administration of the pharmaceutical formulations described herein decrease NT-proBNP levels to normal levels. In some embodiments, the normal level of NT-proBNP is <100 μg/ml.

In some embodiments, administration of the pharmaceutical formulations described herein prevent or reduce pulmonary hypertension Functional Class progression as recognized by the WHO. In some embodiments, administration of the pharmaceutical formulations described herein prevent or reduce pulmonary hypertension Functional Class progression from Functional Class I to Class II pulmonary hypertension as recognized by the WHO. In some embodiments, administration of the pharmaceutical formulations described herein prevent or reduce pulmonary hypertension Functional Class progression from Functional Class II to Class III pulmonary hypertension as recognized by the WHO. In some embodiments, administration of the pharmaceutical formulations described herein prevent or reduce pulmonary hypertension Functional Class progression from Functional Class III to Class IV pulmonary hypertension as recognized by the WHO. In some embodiments, administration of the pharmaceutical formulations described herein promote or increase pulmonary hypertension Functional Class regression as recognized by the WHO. In some embodiments, administration of the pharmaceutical formulations described herein promote or increase pulmonary hypertension Functional Class regression from Class IV to Class III pulmonary hypertension as recognized by the WHO. In some embodiments, administration of the pharmaceutical formulations described herein promote or increase pulmonary hypertension Functional Class regression from Class III to Class II pulmonary hypertension as recognized by the WHO. In some embodiments, administration of the pharmaceutical formulations described herein promote or increase pulmonary hypertension Functional Class regression from Class II to Class I pulmonary hypertension as recognized by the WHO.

In some embodiments, administration of the pharmaceutical formulations described herein improve right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in right ventricular function is due to an increase in ejection fraction. In some embodiments, the improvement in right ventricular function is due to an increase in right ventricular fractional area change and ejection fraction.

In some embodiments, administration of the pharmaceutical formulations described herein improve the pulmonary artery pressure in the patient. In some embodiments, the improvement in pulmonary artery pressure is a reduction in the mean pulmonary artery pressure (mPAP). In some embodiments, administration of the pharmaceutical formulations described herein reduce the mPAP in the patient by at least 10% (e.g., 10%, 15%, 20%, 25%, 30%, 35%, 40%, 45%, or at least 50%). In some embodiments, administration of the pharmaceutical formulations described herein reduce the mPAP by at least 3 mmHg (e.g., at least 3, 5, 7, 10, 12, 15, 20, or 25 mmHg) in the patient. In some embodiments, administration of the pharmaceutical formulations described herein improve the mean right atrial pressure (mRAP) in the patient. In some embodiments, the improvement in the mRAP is a reduction in the mRAP. In some embodiments, administration of the pharmaceutical formulations described herein reduces the mRAP in the patient by at least 10% (e.g., 10%, 15%, 20%, 25%, 30%, 35%, 40%, 45%, or at least 50%). In some embodiments, administration of the pharmaceutical formulations described herein reduce the mRAP by at least 1 mmHg (e.g., at least 1, 2, 3, 4, 5, 6, 7, 8, 9, 10, 11, 12, 13, 14, or 15 mmHg) in the patient.

In some embodiments, the patient has a pulmonary vascular resistance (PVR) greater than or equal to 3 Wood Units. In some embodiments, the patient has a 6-minute walk distance from 150 to 550 meters. In some embodiments, the patient has elevated NT-proBNP levels as compared to a healthy patient. In some embodiments, the patient has a NT-proBNP level of at least 100 μg/mL (e.g., 100, 150, 200, 300, 400, 500, 1000, 3000, 5000, 10,000, 15,000, or 20,000 μg/mL). In some embodiments, the patient has elevated brain natriuretic peptide (BNP) levels as compared to a healthy patient. In some embodiments, the patient has a BNP level of at least 100 μg/mL (e.g., 100, 150, 200, 300, 400, 500, 1000, 3000, 5000, 10,000, 15,000, or 20,000 μg/mL). In some embodiments, administration of the pharmaceutical formulations described herein decrease BNP levels in the patient by at least 10% (e.g., 10%, 15%, 20%, 25%, 30%, 35%, 40%, 45%, 50%, 55% 60%, 65%, 70%, 75%, or at least 80%). In some embodiments, administration of the pharmaceutical formulations described herein decrease BNP levels to normal levels (i.e., <100 μg/ml). In some embodiments, the patient has a mean pulmonary artery pressure (mPAP) selected from the group consisting of: an mPAP of at least 20 mmHg; an mPAP of at least 25 mmHg; an mPAP of at least 30 mmHg; an mPAP of at least 35 mmHg; an mPAP of at least 40 mmHg; an mPAP of at least 45 mmHg; and an mPAP of at least 50 mmHg. In some embodiments, the patient has a mean right atrial pressure (mRAP) selected from the group consisting of: an mRAP of at least 5 mmHg; an mRAP of at least 6 mmHg; an mRAP of at least 8 mmHg; an mRAP of at least 10 mmHg; an mRAP of at least 12 mmHg; an mRAP of at least 14 mmHg; and an mRAP of at least 16 mmHg.

In some embodiments, the PAH is idiopathic pulmonary arterial hypertension (PAH). In some embodiments, the PAH is heritable PAH. In some embodiments, the PAH is drug- or toxin-induced PALL In some embodiments, the PAH is PAH associated with simple, congenital systemic-to-pulmonary shunts at least 1 year following shunt repair. In some embodiments, the patient has Functional Class II or Class III pulmonary hypertension in accordance with the World Health Organization's functional classification system for pulmonary hypertension. In some embodiments, the patient has Functional Class I, Class II, Class III, or Class IV pulmonary hypertension as recognized by the World Health Organization. In some embodiments, the patient has Functional Class I, Class II, Class III, or Class IV pulmonary hypertension in accordance with the World Health Organization's functional classification system for pulmonary hypertension. In some embodiments, the patient has Functional Class IV pulmonary hypertension in accordance with the World Health Organization's functional classification system for pulmonary hypertension. In some embodiments, administration of the pharmaceutical formulations described herein increase transplant free survival in the patient. In some embodiments, administration of the pharmaceutical formulations described herein increase transplant free survival in the patient by at least 10% (e.g., 10%, 15%, 20%, 25%, 30%, 35%, 40%, 45%, or at least 50%). In some embodiments, administration of the pharmaceutical formulations described herein decrease right ventricular hypertrophy in the patient. In some embodiments, administration of the pharmaceutical formulations described herein decrease right ventricular hypertrophy in the patient by at least 10% (e.g., 10%, 15%, 20%, 25%, 30%, 35%, 40%, 45%, or at least 50%). In some embodiments, administration of the pharmaceutical formulations described herein decrease smooth muscle hypertrophy in the patient. In some embodiments, administration of the pharmaceutical formulations described herein decrease smooth muscle hypertrophy in the patient by at least 10% (e.g., 10%, 15%, 20%, 25%, 30%, 35%, 40%, 45%, or at least 50%). In some embodiments, administration of the pharmaceutical formulations described herein decrease pulmonary arteriole muscularity in the patient. In some embodiments, administration of the pharmaceutical formulations described herein decrease pulmonary arteriole muscularity in the patient by at least 10% (e.g., 10%, 15%, 20%, 25%, 30%, 35%, 40%, 45%, or at least 50%).

In some embodiments, administration of the pharmaceutical formulations described herein improve exercise capacity of the patient. In some embodiments, administration of the pharmaceutical formulations described herein decrease the patient's Borg dyspnea index (BDI). In some embodiments, administration of the pharmaceutical formulations described herein reduce the patient's BDI by at least 0.5 index points (e.g., at least 0.5, 1, 1.5, 2, 2.5, 3, 3.5, 4, 4.5, 5, 5.5, 6, 6.5, 7, 7.5, 8, 8.5, 9, 9.5, or 10 index points). In some embodiments, the patient has decreased renal function. In some embodiments, administration of the pharmaceutical formulations described herein further improve renal function. In some embodiments, administration of the pharmaceutical formulations described herein delay clinical worsening of pulmonary arterial hypertension. In some embodiments, administration of the pharmaceutical formulations described herein delay clinical worsening of pulmonary arterial hypertension in accordance with the World Health Organization's functional classification system for pulmonary hypertension. In some embodiments, administration of the pharmaceutical formulations described herein reduce the risk of hospitalization for one or more complications associated with pulmonary arterial hypertension. In some embodiments, administration of the pharmaceutical formulations described herein reduce the risk of morbidity for one or more complications associated with pulmonary arterial hypertension. In some embodiments, the morbidity comprises a change in one or more of the following: increased need for a lung and/or heart transplant; need to initiate rescue therapy with a known treatment for PAH; need to increase prostacyclin by at least 10%; need for atrial septostomy; PAH-specific hospitalization for at least 24 hours; and deterioration of PAH. In some embodiments, the deterioration of PAH comprises a worsening in WHO functional class and a decrease in 6MWD of at least 15%. In some embodiments, administration of the pharmaceutical formulations described herein reduce the risk of death associated with pulmonary arterial hypertension. In some embodiments, administration of the pharmaceutical formulations described herein reduce the risk of death associated with pulmonary arterial hypertension by at least 10% (e.g., 10%, 15%, 20%, 25%, 30%, 35%, 40%, 45%, or at least 50%). In some embodiments, the patient has a hemoglobin level from >8 and <15 g/dl. In some embodiments, the patient's hemoglobin levels are <18 g/dl.

In certain embodiments, the patient treated in accordance with the methods described herein is female. In certain embodiments, the patient treated in accordance with the methods described herein is male. In certain embodiments, the patient treated in accordance with the methods described herein is of any age. In certain embodiments, the patient treated in accordance with the methods described herein is less than 18 years old. In a specific embodiment, the patient treated in accordance with the methods described herein is less than 13 years old. In another specific embodiment, the patient treated in accordance with the methods described herein is less than 12, less than 11, less than 10, less than 9, less than 8, less than 7, less than 6, or less than 5 years old. In another specific embodiment, the patient treated in accordance with the methods described herein is 1-3 years old, 3-5 years old, 5-7 years old, 7-9 years old, 9-11 years old, 11-13 years old, 13-15 years old, 15-20 years old, 20-25 years old, 25-30 years old, or greater than 30 years old. In another specific embodiment, the patient treated in accordance with the methods described herein is 30-35 years old, 35-40 years old, 40-45 years old, 45-50 years old, 50-55 years old, 55-60 years old, or greater than 60 years old. In another specific embodiment, the patient treated in accordance with the methods described herein is 18-64 years old, 65-74 years old, or greater than 75 years old.

In certain embodiments, hemoglobin levels in a patient treated in accordance with the dosage forms and methods provided herein are less than 10 g/dL, 9 g/dL, 8 g/dL, or 7 g/dL. In certain embodiments, hemoglobin levels in a patient treated in accordance with the dosage forms and methods provided herein are between 7 g/dL and 7.5 g/dL, between 7.5 g/dL and 8 g/dL, between 8 g/dL and 8.5 g/dL, between 8.5 g/dL and 9.0 g/dL, between 9.0 g/dL and 9.5 g/dL, or between 9.5 g/dL and 10.0 g/dL.

### Methods of Administration

Described herein are methods of administering the liquid formulations described herein. The liquid formulations described herein can be administered to a patient by parenteral routes such as injection (e.g., subcutaneous, intravenous, intramuscular, intraperitoneal, etc.) or percutaneous, mucosal, nasal, pulmonary or oral administration. In certain embodiments, the liquid formulations are administered subcutaneously.

Also, described herein are methods of administering to a patient any of the liquid formulations described herein comprising administering to the patient any of the liquid formulations described herein with a delivery device, such as a reusable pen or an autoinjector delivery device. In certain embodiments, the liquid formulations are administered using an reusable pen or autoinjector delivery device. In certain embodiments, the liquid formulations described herein are administered using a autoinjector delivery device.

Numerous reusable pen or autoinjector delivery devices can be used to subcutaneously deliver the pharmaceutical formulations of the present invention. Examples include, but are not limited to Molly™ and/or DAI™ and/or PSDI™ (SHL Medical, Zug, Switzerland) and other producers Autopen® (Owen Mumford, Inc., Woodstock, UK), Disetronic Pen (Disetronic Medical Systems, Bergdorf, Switzerland), Humalog® Mix75/25™ pen, Humalog® pen, Humulin® 70/30 pen (Eli Lilly and Co., Indianapolis, Ind.), NovoPen® I, II and III (Novo Nordisk, Copenhagen, Denmark), NovoPen® Junior (Novo Nordisk, Copenhagen, Denmark), BD™ pen (Becton Dickinson, Franklin Lakes, N.J.), OptiPen®, OptiPen Pro®, OptiPen Starlet™, and OptiClik® (Sanofi-Aventis, Frankfurt, Germany). Examples of disposable pen or autoinjector delivery devices having applications in subcutaneous delivery of a pharmaceutical composition of the present invention include, but are not limited to the SoloSTAR® pen (Sanofi-Aventis), the FlexPen® (Novo Nordisk), and the KwikPen™ (Eli Lilly), the SureClick™ Autoinjector (Amgen, Thousand Oaks, Calif), the Penlet® (Haselmeier, Stuttgart, Germany), the EpiPen® (Dey, L. P.), and the Humira® Pen (Abbott Labs, Abbott Park, Ill.).

In certain embodiments, the liquid formulations described herein are administered using a reusable pen or autoinjector delivery device wherein the reusable pen or autoinjector delivery devices comprises a pre-filled syringe. In certain embodiments, the capacity of the pre-filled syringe is selected from a 0.5 ml to 10 ml syringe. In certain embodiments, the capacity of the pre-filled syringe is 0.5 ml, 0.75 ml, 1.0 ml, 1.25 ml, 1.5 ml, 1.75 ml, 2.0 ml, 2.25 ml, 2.5 ml, 2.75 ml, 3.0 ml, 3.25 ml, 3.5 ml, 3.75 ml, 4.0 ml, 4.25 ml, 4.5 ml, 4.75 ml, 5.0 ml, 5.25 ml, 5.5 ml, 5.75 ml, 6.0 ml, 6.25 ml, 6.5 ml, 6.75 ml, 7.0 ml, 7.25 ml, 7.5 ml, 7.75 ml, 8.0 ml, 8.25 ml, 8.5 ml, 8.75 ml, 9.0 ml, 9.25 ml, 9.5 ml, 9.75 ml, 10.0 ml syringe pre filled any of the liquid formulations described herein.

In certain embodiments, the prefilled syringe contains any of the formulations described herein wherein the concentration of the ActRIIa protein is between 5 mg/ml to 100 mg/ml. In certain embodiments, the concentration of the ActRIIa protein is between 5 mg/ml to 50 mg/ml. In certain embodiments, the concentration of the ActRIIa protein is between 8.3 mg/ml to 50 mg/ml. In certain embodiments, the concentration of the ActRIIa protein is 5 mg/ml, 10 mg/ml, 15 mg/ml, 20 mg/ml, 25 mg/ml, 30 mg/ml, 35 mg/ml, 40 mg/ml, 45 mg/ml, 50 mg/ml, 55 mg/ml, 60 mg/ml, 65 mg/ml, 70 mg/ml, 75 mg/ml, 80 mg/ml, 85 mg/ml, 90 mg/ml, 95 mg/ml, or 100 mg/ml.

In one embodiment, the reusable pen or autoinjector delivery device contains a needle with a gauge that is 27 G or smaller in diameter. In one embodiment, the needle gauge ranges in size from 25 G to 33 G (including ranges intermediate thereto, e.g., 25 sG, 26, 26 sG, 27 G, 28 G, 29 G, 30 G, 31 G, 32 G, and 33 G). In one embodiment, the smallest needle diameter and appropriate length is chosen in accordance with the viscosity characteristics of the formulation and the device used to deliver the formulation of the invention.

In certain embodiments, the liquid formulations described herein are administered using a reusable pen or autoinjector delivery device wherein the delivered volume of any of liquid formulations described herein is 1 ml to 2 ml. In certain embodiments, the delivered volume of any of the liquid formulations described herein is 1.0 ml, 1.1 ml, 1.2 ml, 1.3 ml, 1.4 ml, 1.5 ml, 1.6 ml, 1.7 ml, 1.8 ml, 1.9 ml or 2.0 ml. In certain embodiments, the delivered volume of any of the liquid formulations described herein is 1.8 ml.

In certain embodiments, the reusable pen or autoinjector delivery device is useful for the injection of any of the liquid formulations described herein within a range of 1-5 seconds. In certain embodiments, the reusable pen or autoinjector delivery device is useful for the injection of any of the liquid formulations described herein within a range of 1-4 seconds. In certain embodiments, the reusable pen or autoinjector delivery device is useful for the injection any of the liquid formulations described herein within a range of 1-3 seconds. In certain embodiments, the reusable pen or autoinjector delivery device injects allows the injection of any of the liquid formulations described herein within a range of 2-3 seconds. In certain embodiments, the reusable pen or autoinjector delivery device allows the injection of any of the liquid formulations described herein within a range of 2-4 seconds. In certain embodiments, the reusable pen or autoinjector delivery device allows the injection of any of the liquid formulations described within 1, 2, 3, 4 or 5 second(s).

In certain embodiments, the reusable pen or autoinjector delivery device is useful for the injection of any of the liquid formulations described herein with a spring force between 10N to 100N. In certain embodiments, the reusable pen or autoinjector delivery device is useful for the injection of any of the liquid formulations described herein with a spring force between 10N to 50N. In certain embodiments, the reusable pen or autoinjector delivery device is useful for the injection of any of the liquid formulations described herein with a spring force between of 10N, 20N, 30N, 40N, 50N, 60N, 70N, 80N, 90N or 100N. In certain embodiments, the reusable pen or autoinjector delivery device is useful for the injection of any of the liquid formulation described herein with a spring force between about TON, 20N, 30N, 40N, 50N, 60N, 70N, 80N, 90N or 100N. In certain embodiments, the reusable pen or autoinjector delivery device is useful for the injection of any of the liquid formulations described herein with a spring force of about 50N.

In certain embodiments, the reusable pen or autoinjector delivery device allows for the injection any of the liquid formulations described herein with a extrusion force between 1N-TON. In certain embodiments, the reusable pen or autoinjector delivery device allows for the injection of any of the liquid formulations described herein with a extrusion force of TN, 2N, 3N, 4N, 5N, 6N, 7N, 8N, 9N or TON. In certain embodiments, the reusable pen or autoinjector delivery device allows for the injection of any of the liquid formulations described herein with a extrusion force of about TN, 2N, 3N, 4N, 5N, 6N, 7N, 8N, 9N or TON. In certain embodiments, the reusable pen or autoinjector delivery device allows for the injection of any of the liquid formulations described herein with a extrusion force of about 6N.

### EXAMPLES

The disclosure now being generally described, will be more readily understood by reference to the following examples, which are included merely for purposes of illustration of certain embodiments of the present disclosure, and are not intended to limit the disclosure.

The meanings of the abbreviations in Examples are shown below.
CHO=Chinese hamster overy
SDS PAGE=sodium dodecyl sulfate-polyacrylamide gel electrophoresis
HMW=high molecular weight species
LMW=low molecular weight species
PS80=polysorbate 80
DTPA=Diethylenetriamine pentaacetate
EDTA=Ethylenediaminetetraacetic acid
MFI=Mean Fluorescence Intensity
UPSEC=Ultra-high-performance size-exclusion chromatography
cIEF=Capillary isoelectric focusing
CE-SDS=Capillary electrophoresis sodium dodecyl sulfate
Fc=fragment crystallizable region
IgG=Immunoglobulin G
P188=poloxamer 188

### Example 1 ActRIIA-Fc Fusion Proteins

A soluble ActRIIA fusion protein was constructed that has the extracellular domain of human ActRIIA fused to a human or mouse Fc domain with a minimal linker in between. The constructs are referred to as ActRIIA-hFc and ActRIIA-mFc, respectively.

The ActRIIA-hFc shown below is also known as sotatercept and as purified from CHO cell lines (SEQ ID NO: 32):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK

The ActRIIA-hFc and ActRIIA-mFc proteins were expressed in CHO cell lines. Three different leader sequences were considered:

(i) Honey bee melittin (HBML):
(SEQ ID NO: 33)
MKFLVNVALVFMVVYISYIYA
(ii) Tissue plasminogen activator (TPA):
(SEQ ID NO: 34)
MDAMKRGLCCVLLLCGAVFVSP
(iii) Native:
(SEQ ID NO: 35)
MGAAAKLAFAVFLISCSSGA

The selected form employs the TPA leader and has the following unprocessed amino acid sequence:

(SEQ ID NO: 36)
MDAMKRGLCCVLLLCGAVFVSPGAAILGRSETQECLFFNANWEKDRINQT
GVEPCYGDKDKRRHCFATWKNISGSIEIVKQGCWLDDINCYDRTDCVEKK
DSPEVYFCCCEGNMCNEKFSYFPEMEVTQPTSNPVTPKPPTGGGTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PVPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK

This protein is encoded by the following nucleic acid sequence:

(SEQ ID NO: 37)
ATGGATGCAATGAAGAGAGGGCTCTGCTGTGTGCTGCTGCTGTGTGGAGC
AGTCTTCGTTTCGCCCGGCGCCGCTATACTTGGTAGATCAGAAACTCAGG
AGTGTCTTTTTTTAATGCTAATTGGGAAAAAGACAGAACCAATCAAACTG
GTGTTGAACCGTGTTATGGTGACAAAGATAAACGGCGGCATTGTTTTGCT
ACCTGGAAGAATATTTCTGGTTCCATTGAATAGTGAAACAAGGTTGTTGG
CTGGATGATATCAACTGCTATGACAGGACTGATTGTGTAGAAAAAAAAGA
CAGCCCTGAAGTATATTTCTGTTGCTGTGAGGGCAATATGTGTAATGAAA
AGTTTTCTTATTTTCCGGAGATGGAAGTCACACAGCCCACTTCAAATCCA
GTTACACCTAAGCCACCCACCGGTGGTGGAACTCACACATGCCCACCGTG
CCCAGCACCTGAACTCCTGGGGGGACCGTCAGTCTTCCTCTTCCCCCCAA
AACCCAAGGACACCCTCATGATCTCCCGGACCCCTGAGGTCACATGCGTG
GTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGT
ACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCC
AGTCCCCATCGAGAAAACCATCTCCAAAGCCAAAGGGCAGCCCCGAGAAC
CACAGGTGTACACCCTGCCCCCATCCCGGGAGGAGATGACCAAGAACCAG
GTCAGCCTGACCTGCCTGGTCAAAGGCTTCTATCCCAGCGACATCGCCGT
GGAGTGGGAGAGCAATGGGCAGCCGGAGAACAACTACAAGACCACGCCTC
CCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCA
TGAGGCTCTGCACAACCACTACACGCAGAAGAGCCTCTCCCTGTCTCCGG
GTAAATGAGAATTC

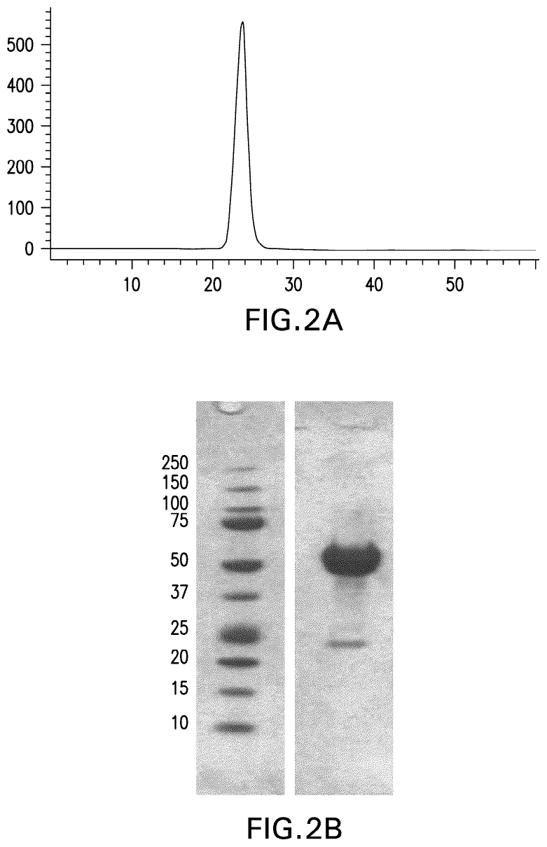

Both ActRIIA-hFc and ActRIIA-mFc were remarkably amenable to recombinant expression. As shown in FIGS. 2A and 2B, the protein was purified as a single, well-defined peak of protein. N-terminal sequencing revealed a single sequence of -ILGRSETQE (SEQ ID NO: 38). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

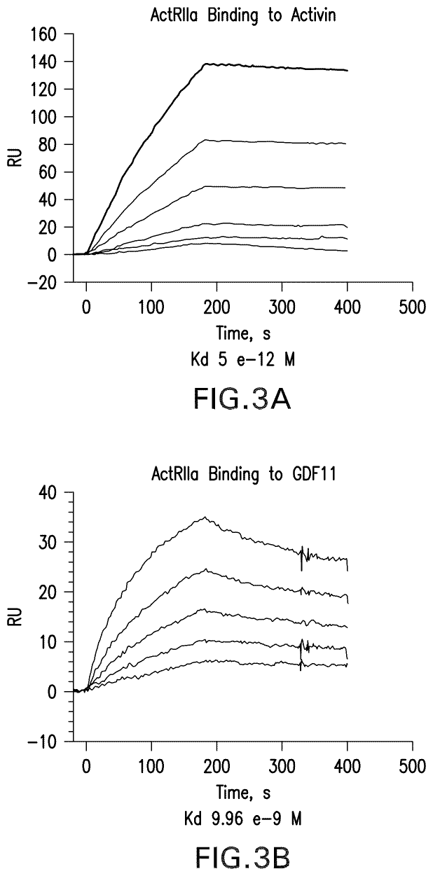

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIGS. 3A and 3B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2 Characterization of an ActRIIA-hFc Protein

ActRIIA-hFc fusion protein was expressed in stably transfected CHO-DUKX B11 cells from a pAID4 vector (SV40 on/enhancer, CMV promoter), using a tissue plasminogen leader sequence of SEQ ID NO: 34. The protein, purified as described above in Example 1, had a sequence of SEQ ID NO: 32. The Fc portion is a human IgG1 Fc sequence, as shown in SEQ ID NO: 32. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells (see, del Re et al. (2004) J Biol Chem. 279(51):53126-53135). Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

Additional ActRIIA Ligand Traps (ActRIIA-Fc fusion proteins modified so as to reduce the ratio of activin A binding relative to myostatin or GDF11) are described in International Patent Application Publication Nos. WO 2006/012627 and WO 2007/062188, incorporated by reference herein.

### Example 3 Preparation of a Liquid ActRIIA-hFc Fusion Protein Formulation

Initially the ActRIIA-hFc fusion protein sotatercept (SEQ ID NO: 32) was formulated in phosphate buffered saline as a frozen solution. Based on further development studies a lyophilized citrate buffer formulation of the ActRIIA-hFc fusion protein sotatercept was developed containing sucrose and polysorbate 80, to enable a stable formulation with adequate shelf life for commercialization. The lyophilized formulation consists of a lyophilized cake of sotatercept (45 mg/vial or 60 mg/vial) as part of an injection kit that also contains of vial adapter, prefilled syringe of Sterile Water for Injection, an injection syringe, an injection needle and alcohol swabs. However, users disliked the need to reconstitute the lyophilized cake prior to administration. To improve the user experience, a liquid formulation comprising sotatercept, suitable for a prefilled syringe and/or an autoinjector device combination was formulated in light of the following experiments.

### Concentration Liquid Feasibility Studies

A concentration stability study was conducted to evaluate the short-term biophysical stability of sotatercept (SEQ ID NO:32) at various concentrations. Formulations comprising 100 mg/mL and 50 mg/mL sotatercept in 10 mM citrate buffer and 8% (weight/volume) sucrose at pH 5.8 were prepared. Table 1 shows the stability of the formulation containing 100 mg/mL of sotatercept. The formulation containing 100 mg/mL of sotatercept was staged at different temperatures (5° C., 25° C. and 40° C.) and selected timepoints were analyzed to show that the formulation in citrate buffer and sucrose had minimal change in high molecular weight formation (as measured by UPSEC) and charge distribution (as measured by cIEF) at 5° C. and 25° C.

TABLE 1
Sotatercept High Concentration Feasibility Stability (10 mM Citrate pH 5.8)

| | 100 mg/mL Sotatercept in 10 mM Citrate, 8% (weight/volume) Sucrose, pH 5.8 | | | | | |
|---|---|---|---|---|---|---|
| | | 5° C. | | 25° C. | | 40° C. |
| Assay | Initial | 3 M | 5 M | 3 M | 5 M | 1 M |

UP-SEC

| | | | | | | |
|---|---|---|---|---|---|---|
| % HMW | 0.9 | 1.0 | 1.0 | 1.5 | 1.6 | 1.9 |
| % Monomer | 98.6 | 98.5 | 98.5 | 97.5 | 97.1 | 96.8 |
| % LMW | 0.5 | 0.5 | 0.5 | 1.0 | 1.2 | 1.3 |

cIEF

| | | | | | | |
|---|---|---|---|---|---|---|
| % Acidic | 18.122 | 27.992 | NT | 28.004 | NT | 19.083 |
| % Main 1 | 40.882 | 31.976 | NT | 30.222 | NT | 32.921 |
| % Main 2 | 30.415 | 35.064 | NT | 33.402 | NT | 28.550 |
| % Basic | 10.583 | 4.968 | NT | 8.372 | NT | 19.447 |

Tables 2 and 3 show the stability of the formulation containing 50 mg/mL of sotatercept. The formulation containing 50 mg/mL of sotatercept was staged at different temperatures (5° C., 25° C. and 40° C.) and selected timepoints were analyzed to show that the formulation in citrate buffer, sucrose, PS80 and with or without DTPA, has minimal change in subvisible particle formation (as measured by MFI), high molecular weight formation (as measured by UPSEC), charge distribution (as measured by cIEF) and fragmentation (as measured by CE-SDS) at 5° C. and 25° C.

TABLE 2
Sotatercept Liquid Formulation Long Term Stability (10 mM Citrate pH 5.8)

| | 50 mg/mL Sotatercept in 10 mM Citrate, 8% (weight/volume) Sucrose, 0.02% (weight/volume) PS-80, pH 5.8 | | | | | | | | | | | |
|---|---|---|---|---|---|---|---|---|---|---|---|---|
| | | | 5° C. | | | | | 25° C. | | | 40° C. | |
| Assay | Initial | 1 M | 3 M | 6 M | 12 M | 18 M | 24 M | 3 M | 6 M | 18 M | 1 M | 3 M |
| pH | 6.1 | 5.9 | 6.0 | NT | NT | NT | NT | 6.0 | NT | NT | 6.0 | 6.0 |

UP-SEC

| | | | | | | | | | | | | |
|---|---|---|---|---|---|---|---|---|---|---|---|---|
| % HMW | 0.6 | 0.6 | 0.7 | NT | NT | 0.8 | 0.9 | 0.9 | 1.0 | 1.4 | 1.1 | 1.6 |
| % Monomer | 98.9 | 98.8 | 98.7 | NT | NT | 98.5 | 97.2 | 98.1 | 97.5 | 95.1 | 97.4 | 95.1 |
| % LMW | 0.6 | 0.6 | 0.6 | NT | NT | 0.7 | 1.9 | 0.9 | 0.4 | 3.5 | 1.5 | 3.3 |

cIEF

| | | | | | | | | | | | | |
|---|---|---|---|---|---|---|---|---|---|---|---|---|
| % Acidic | 19.0 | 21.3 | 19.2 | NT | NT | 19.1 | 21.4 | 20.8 | 20.5 | 21.0 | 17.5 | 22.5 |
| % Main 1 | 27.8 | 28.8 | 28.3 | NT | NT | 40.9 | 41.0 | 30.3 | 35.3 | 29.4 | 25.3 | 37.5 |
| % Main 2 | 37.2 | 36.3 | 38.5 | NT | NT | 28.3 | 27.8 | 32.7 | 29.3 | 26.2 | 34.3 | 15.6 |
| % Basic | 16.1 | 13.6 | 14.0 | NT | NT | 11.7 | 9.9 | 16.3 | 14.9 | 23.4 | 22.9 | 24.4 |

CE-SDS (NR)

| | | | | | | | | | | | | |
|---|---|---|---|---|---|---|---|---|---|---|---|---|
| % Main IgG | 97.3 | 96.6 | NT | NT | NT | 98.2 | 98.1 | NT | 96.4 | 94.9 | 96.1 | NT |
| % LMW | 2.5 | 3.0 | NT | NT | NT | 1.8 | 1.9 | NT | 3.1 | 5.1 | 3.5 | NT |

MFI

| | | | | | | | | | | | | |
|---|---|---|---|---|---|---|---|---|---|---|---|---|
| ≥10 μm/mL | 123 | 40 | 23 | NT | NT | NT | NT | 33 | NT | NT | 35 | 5 |
| ≥25 μm/mL | 3 | 0 | 3 | NT | NT | NT | NT | 0 | NT | NT | 0 | 3 |

TABLE 3
Sotatercept Liquid Formulation Long Term Stability (10 mM Citrate pH 5.8)

| | 50 mg/mL Sotatercept in 10 mM Citrate, 8% (weight/volume) Sucrose, 0.02% (weight/volume) PS-80, 50 μM DTPA, pH 5.8 | | | | | | | | |
|---|---|---|---|---|---|---|---|---|---|
| | | 5° C. | | | | 25° C. | | 40° C. | |
| Assay | Initial | 1 M | 3 M | 6 M | 12 M | 3 M | 6 M | 1 M | 3 M |
| pH | 6.1 | 5.9 | 6.0 | NT | NT | 6.0 | | 5.9 | 5.9 |

UP-SEC

| | | | | | | | | | |
|---|---|---|---|---|---|---|---|---|---|
| % HMW | 0.6 | 0.6 | 0.7 | NT | 0.8 | 0.9 | 1.0 | 1.1 | 1.6 |
| % Monomer | 98.9 | 98.8 | 98.8 | NT | 98.6 | 98.1 | 97.3 | 97.4 | 95.0 |
| % LMW | 0.6 | 0.6 | 0.6 | NT | 0.6 | 1.0 | 0.5 | 1.5 | 3.4 |

cIEF

| | | | | | | | | | |
|---|---|---|---|---|---|---|---|---|---|
| % Acidic | 18.5 | 20.7 | 20.1 | NT | NT | 19.8 | 20.8 | 21.4 | 24.7 |
| % Main 1 | 27.7 | 30.5 | 28.4 | NT | NT | 30.2 | 36.2 | 26.8 | 36.8 |
| % Main 2 | 37.7 | 34.9 | 38.0 | NT | NT | 33.2 | 29.8 | 30.9 | 15.3 |
| % Basic | 16.2 | 13.9 | 13.5 | NT | NT | 16.9 | 13.3 | 20.9 | 23.3 |

CE-SDS (NR)

| | | | | | | | | | |
|---|---|---|---|---|---|---|---|---|---|
| % Main IgG | 97.9 | 96.9 | NT | NT | NT | NT | 96.6 | 96.6 | NT |
| % LMW | 2.0 | 2.3 | NT | NT | NT | NT | 3.0 | 2.8 | NT |

MFI

| | | | | | | | | | |
|---|---|---|---|---|---|---|---|---|---|
| ≥10 μm/mL | 83 | 20 | 15 | NT | 7 | 13 | NT | 18 | 23 |
| ≥25 μm/mL | 8 | 0 | 0 | NT | 0 | 5 | NT | 3 | 0 |

### pH and Buffer Feasibility Studies

The behavior of sotatercept (SEQ ID NO: 32) was analyzed in 6-buffers at differing pH and 8% sucrose. The buffers tested were histidine at pH 6.5 and 7; phosphate at pH 5, 5.5, 6, 6.5 and 7; citrate at pH 4.5, 5, 5.5, 6, 6.5 and 7; acetate at pH 4.5, 5, 5.5 and 6; succinate 4.5 and 5; and glutamate at pH 4.5 and 5. The results are shown in in Tables 4-13. The formulations were thermally stressed at 40° C. for up to two months and tested for biophysical stability (turbidity), subvisible particle formation (measured by MFI), high molecular weight formation (via UPSEC), fragmentation (via R-CESDS and NR-CESDS) and charge distribution (via cIEF).

TABLE 4
Sotatercept Liquid Formulation Feasibility Stability (10 mM Histidine)

| | 50 mg/mL Sotatercept in 10 mM Histidine, 8% (weight/volume) Sucrose, pH 6.5-7.0 | | | | | |
|---|---|---|---|---|---|---|
| | pH 6.5 | | | pH 7.0 | | |
| | | 40° C. | | | 40° C. | |
| Assay | Initial | 1 M | 2 M | Initial | 1 M | 2 M |
| Conc. (mg/mL) | 47.25 | 47.52 | 47.26 | 47.25 | 47.33 | 46.77 |
| pH | 6.02 | 6.26 | 6.21 | 6.25 | 6.52 | 6.46 |
| Turbidity A₃₅₀₋₅₀₀ | 0.126 | 0.169 | 0.203 | 0.125 | 0.171 | 0.206 |

UP-SEC

| | | | | | | |
|---|---|---|---|---|---|---|
| % HMW | 0.6 | 0.9 | 1.0 | 0.6 | 0.8 | 1.0 |
| % Monomer | 98.9 | 97.6 | 96.5 | 98.9 | 97.6 | 96.3 |
| % LMW | 0.1 | 0.5 | 0.8 | 0.1 | 0.5 | 0.8 |

cIEF

| | | | | | | |
|---|---|---|---|---|---|---|
| % Acidic | 22.241 | 22.014 | 21.063 | 21.474 | 23.526 | 24.243 |
| % Main 1 | 39.017 | 31.914 | 29.056 | 35.865 | 32.761 | 28.640 |
| % Main 2 | 31.467 | 28.571 | 27.758 | 29.061 | 30.525 | 29.344 |
| % Basic | 7.275 | 17.501 | 22.124 | 13.599 | 13.188 | 17.772 |

CE-SDS (NR)

| | | | | | | |
|---|---|---|---|---|---|---|
| % Main IgG | NT | NT | 95.261 | NT | NT | NT |
| % LMW | NT | NT | 4.505 | NT | NT | NT |

CE-SDS (R)

| | | | | | | |
|---|---|---|---|---|---|---|
| % Total Purity | NT | NT | 94.280 | NT | NT | NT |
| % Impurity | NT | NT | 5.720 | NT | NT | NT |

MFI

| | | | | | | |
|---|---|---|---|---|---|---|
| ≥10 μm/mL | 3 | 60 | 19 | 10 | 14 | 7 |
| ≥25 μm/mL | 0 | 14 | 7 | 0 | 3 | 0 |

TABLE 5
Sotatercept Liquid Formulation Feasibility Stability (10 mM Histidine)

| | 50 mg/mL Sotatercept in 10 mM Histidine, 8% (weight/volume) Sucrose, pH 5.0-6.0 | | | | | | | | |
|---|---|---|---|---|---|---|---|---|---|
| | pH 5.0 | | | pH 5.5 | | | pH 6.0 | | |
| | | 40° C. | | | 40° C. | | | 40° C. | |
| Assay | Initial | 1 M | 2 M | Initial | 1 M | 2 M | Initial | 1 M | 2 M |
| Conc. (mg/mL) | 46.16 | 46.21 | 47.17 | 47.45 | 47.25 | 47.51 | 46.33 | 47.35 | 47.59 |
| pH | 5.17 | 5.47 | 5.41 | 5.41 | 5.69 | 5.62 | 5.65 | 6.17 | 5.94 |
| Turbidity A₃₅₀₋₅₀₀ | 0.138 | 0.167 | 0.207 | 0.133 | 0.167 | 0.204 | 0.128 | 0.134 | 0.203 |

UP-SEC

| | | | | | | | | | |
|---|---|---|---|---|---|---|---|---|---|
| % HMW | 0.6 | 1.5 | 2.2 | 0.6 | 1.1 | 1.7 | 0.6 | 1.0 | 1.2 |
| % Monomer | 98.8 | 97.5 | 96.0 | 98.9 | 97.8 | 96.6 | 98.9 | 97.8 | 96.8 |
| % LMW | 0.1 | 0.7 | 1.3 | 0.1 | 0.7 | 1.1 | 0.1 | 0.6 | 0.9 |

cIEF

| | | | | | | | | | |
|---|---|---|---|---|---|---|---|---|---|
| % Acidic | 21.621 | 14.210 | 11.335 | 22.478 | 16.483 | 14.072 | 22.663 | 18.935 | 17.487 |
| % Main 1 | 39.432 | 28.156 | 23.975 | 38.506 | 30.466 | 25.766 | 38.368 | 32.272 | 28.854 |
| % Main 2 | 30.669 | 21.287 | 18.277 | 31.556 | 24.300 | 21.370 | 31.139 | 26.521 | 23.999 |
| % Basic | 8.278 | 36.348 | 46.413 | 7.460 | 28.751 | 38.792 | 7.830 | 22.272 | 29.660 |

CE-SDS (NR)

| | | | | | | | | | |
|---|---|---|---|---|---|---|---|---|---|
| % Main IgG | NT | NT | NT | NT | NT | NT | NT | NT | 95.102 |
| % LMW | NT | NT | NT | NT | NT | NT | NT | NT | 4.606 |

CE-SDS (R)

| | | | | | | | | | |
|---|---|---|---|---|---|---|---|---|---|
| % Total Purity | NT | NT | NT | NT | NT | NT | NT | NT | 88.787 |
| % Impurity | NT | NT | NT | NT | NT | NT | NT | NT | 11.213 |

MFI

| | | | | | | | | | |
|---|---|---|---|---|---|---|---|---|---|
| ≥10 μm/mL | 30 | 324 | 49 | 26 | 40 | 26 | 17 | 26 | 10 |
| ≥25 μm/mL | 0 | 44 | 5 | 0 | 12 | 10 | 0 | 10 | 5 |

TABLE 6
Sotatercept Liquid Formulation Feasibility Stability (10 mM Phosphate)

| | 50 mg/mL Sotatercept in 10 mM Phosphate, 8% (weight/volume) Sucrose, pH 5.0-6.0 | | | | | | | | |
|---|---|---|---|---|---|---|---|---|---|
| | pH 5.0 | | | pH 5.5 | | | pH 6.0 | | |
| | | 40° C. | | | 40° C. | | | 40° C. | |
| Assay | Initial | 1 M | 2 M | Initial | 1 M | 2 M | Initial | 1 M | 2 M |
| Conc. (mg/mL) | 46.59 | 47.01 | 47.40 | 47.40 | 47.60 | 48.76 | 48.22 | 47.81 | 49.07 |
| pH | 5.51 | 5.90 | 5.83 | 5.66 | 5.90 | 5.84 | 5.85 | 6.05 | 6.04 |
| Turbidity A₃₅₀₋₅₀₀ | 0.134 | 0.138 | 0.147 | 0.131 | 0.139 | 0.144 | 0.131 | 0.141 | 0.142 |

UP-SEC

| | | | | | | | | | |
|---|---|---|---|---|---|---|---|---|---|
| % HMW | 0.6 | 1.1 | 1.6 | 0.6 | 1.1 | 1.5 | 0.6 | 1.1 | 1.3 |
| % Monomer | 98.8 | 97.7 | 96.6 | 98.8 | 97.6 | 96.4 | 98.8 | 97.5 | 96.3 |
| % LMW | 0.1 | 0.5 | 0.9 | 0.1 | 0.5 | 0.9 | 0.1 | 0.5 | 0.8 |

cIEF

| | | | | | | | | | |
|---|---|---|---|---|---|---|---|---|---|
| % Acidic | 21.970 | 19.304 | 17.829 | 21.842 | 20.225 | 19.274 | 21.728 | 21.044 | 20.631 |
| % Main 1 | 38.962 | 32.517 | 29.980 | 39.569 | 32.472 | 29.516 | 39.110 | 31.535 | 29.784 |
| % Main 2 | 31.097 | 27.007 | 25.058 | 30.953 | 27.557 | 26.688 | 30.675 | 27.772 | 27.663 |
| % Basic | 7.971 | 21.172 | 27.133 | 7.636 | 19.746 | 24.522 | 8.487 | 19.649 | 21.923 |

CE-SDS (NR)

| | | | | | | | | | |
|---|---|---|---|---|---|---|---|---|---|
| % Main IgG | NT | NT | 94.922 | NT | NT | 95.285 | NT | NT | 95.043 |
| % LMW | NT | NT | 4.695 | NT | NT | 4.360 | NT | NT | 4.568 |

CE-SDS (R)

| | | | | | | | | | |
|---|---|---|---|---|---|---|---|---|---|
| % Total Purity | NT | NT | 88.965 | NT | NT | 89.071 | NT | NT | 93.886 |
| % Impurity | NT | NT | 11.035 | NT | NT | 10.929 | NT | NT | 6.114 |

MFI

| | | | | | | | | | |
|---|---|---|---|---|---|---|---|---|---|
| ≥10 μm/mL | 37 | 23 | 30 | 10 | 10 | 21 | 23 | 33 | 17 |
| ≥25 μm/mL | 5 | 3 | 3 | 0 | 0 | 0 | 0 | 3 | 0 |

TABLE 7
Sotatercept Liquid Formulation Feasibility Stability (10 mM Phosphate)

| | 50 mg/mL Sotatercept in 10 mM Phosphate, 8% (weight/volume) Sucrose, pH 6.5-7.0 | | | | | |
|---|---|---|---|---|---|---|
| | pH 6.5 | | | 7.0 | | |
| | | 40° C. | | | 40° | |
| Assay | Initial | 1 M | 2 M | Initial | 1 M | 2 M |
| Conc. (mg/mL) | 47.02 | 47.75 | 47.45 | 47.91 | 48.50 | 24.04 |
| pH | 6.27 | 6.42 | 6.44 | 6.78 | 6.96 | 6.86 |
| Turbidity A₃₅₀₋₅₀₀ | 0.130 | 0.136 | 0.138 | 0.130 | 0.138 | 0.145 |

UP-SEC

| | | | | | | |
|---|---|---|---|---|---|---|
| % HMW | 0.7 | 1.0 | 1.2 | 0.7 | 1.1 | 1.3 |
| % Monomer | 98.8 | 97.2 | 95.8 | 98.7 | 96.5 | 94.4 |
| % LMW | 0.1 | 0.5 | 0.9 | 0.1 | 0.6 | 1.2 |

cIEF

| | | | | | | |
|---|---|---|---|---|---|---|
| % Acidic | 21.743 | 24.243 | 26.137 | 22.782 | 29.561 | 35.024 |
| % Main 1 | 39.014 | 33.323 | 28.442 | 37.878 | 29.693 | 22.660 |
| % Main 2 | 31.010 | 30.987 | 30.661 | 31.624 | 32.983 | 31.191 |
| % Basic | 8.233 | 11.447 | 14.760 | 7.717 | 7.763 | 11.124 |

CE-SDS (NR)

| | | | | | | |
|---|---|---|---|---|---|---|
| % Main IgG | NT | NT | 95.031 | NT | NT | 93.447 |
| % LMW | NT | NT | 4.703 | NT | NT | 6.286 |

CE-SDS (R)

| | | | | | | |
|---|---|---|---|---|---|---|
| % Total Purity | NT | NT | 93.750 | NT | NT | 93.739 |
| % Impurity | NT | NT | 6.250 | NT | NT | 6.261 |

MFI

| | | | | | | |
|---|---|---|---|---|---|---|
| ≥10 μm | 35 | 14 | 5 | 17 | 5 | 10 |
| ≥25 μm | 0 | 3 | 0 | 0 | 0 | 0 |

TABLE 8
Sotatercept Liquid Formulation Feasibility Stability (10 mM Citrate)

| | 50 mg/mL Sotatercept in 10 mM Citrate, 8% (weight/volume) Sucrose, pH 4.5-5.5 | | | | | | | | |
|---|---|---|---|---|---|---|---|---|---|
| | pH 4.5 | | | pH 5.0 | | | pH 5.5 | | |
| | | 40° C. | | | 40° C. | | | 40° C. | |
| Assay | Initial | 1 M | 2 M | Initial | 1 M | 2 M | Initial | 1 M | 2 M |
| Conc. (mg/mL) | 44.58 | 46.18 | 46.03 | 46.68 | 46.92 | 46.27 | 47.20 | 47.44 | 46.77 |
| pH | 4.69 | 4.92 | 4.86 | 5.22 | 5.43 | 5.39 | 5.61 | 5.75 | 5.578 |
| Turbidity A₃₅₀₋₅₀₀ | 0.144 | 0.157 | 0.187 | 0.135 | 0.144 | 0.169 | 0.133 | 0.139 | 0.150 |

UP-SEC

| | | | | | | | | | |
|---|---|---|---|---|---|---|---|---|---|
| % HMW | 0.8 | 2.3 | 3.6 | 0.6 | 1.4 | 1.9 | 0.6 | 1.0 | 1.3 |
| % Monomer | 98.6 | 96.1 | 94.0 | 98.8 | 97.5 | 96.9 | 98.9 | 97.7 | 96.8 |
| % LMW | 0.1 | 1.4 | 2.1 | 0.1 | 0.8 | 0.8 | 0.1 | 0.6 | 0.9 |

cIEF

| | | | | | | | | | |
|---|---|---|---|---|---|---|---|---|---|
| % Acidic | 21.107 | 12.360 | 12.220 | 21.969 | 16.280 | 15.834 | 21.830 | 20.637 | 20.773 |
| % Main 1 | 39.835 | 22.544 | 19.517 | 39.136 | 28.539 | 22.747 | 39.464 | 32.118 | 28.193 |
| % Main 2 | 30.278 | 16.429 | 15.006 | 31.203 | 21.377 | 19.649 | 31.018 | 26.108 | 25.434 |
| % Basic | 8.780 | 48.666 | 53.258 | 7.691 | 33.804 | 41.771 | 7.688 | 21.137 | 25.600 |

CE-SDS (NR)

| | | | | | | | | | |
|---|---|---|---|---|---|---|---|---|---|
| % Main IgG | NT | NT | 88.219 | NT | NT | 92.980 | NT | NT | 94.994 |
| % LMW | NT | NT | 10.633 | NT | NT | 6.385 | NT | NT | 4.554 |

CE-SDS (R)

| | | | | | | | | | |
|---|---|---|---|---|---|---|---|---|---|
| % Total Purity | NT | NT | 70.912 | NT | NT | 87.825 | NT | NT | 91.937 |
| % Impurity | NT | NT | 29.088 | NT | NT | 12.175 | NT | NT | 8.063 |

MFI

| | | | | | | | | | |
|---|---|---|---|---|---|---|---|---|---|
| ≥10 μm/mL | 21 | 543 | 86 | 12 | 12 | 240 | 3 | 7 | 23 |
| ≥25 μm/mL | 0 | 95 | 12 | 0 | 3 | 35 | 0 | 3 | 3 |

TABLE 9
Sotatercept Liquid Formulation Feasibility Stability (10 mM Citrate)

| | 50 mg/mL Sotatercept in 10 mM Citrate, 8% (weight/volume) Sucrose, pH 6.0-7.0 | | | | | | | | |
|---|---|---|---|---|---|---|---|---|---|
| | pH 6.0 | | | pH 6.5 | | | pH 7.0 | | |
| | | 40° C. | | | 40° C. | | | 40° C. | |
| Assay | Initial | 1 M | 2 M | Initial | 1 M | 2 M | Initial | 1 M | 2 M |
| Conc. | 47.63 | 47.39 | | 47.13 | 47.16 | | 46.68 | 47.49 | |
| (mg/mL) | | | | | | | | | |
| pH | 6.02 | 6.18 | 6.33 | 6.40 | 6.58 | 6.48 | 6.75 | 6.82 | 6.78 |
| Turbidity | 0.132 | 0.137 | 0.144 | 0.132 | 0.135 | 0.148 | 0.131 | 0.138 | 0.144 |
| A₃₅₀₋₅₀₀ | | | | | | | | | |

UP-SEC

| | | | | | | | | | |
|---|---|---|---|---|---|---|---|---|---|
| % HMW | 0.6 | 1.0 | 1.2 | 0.6 | 1.0 | 1.2 | 0.7 | 1.0 | 1.2 |
| % Monomer | 98.8 | 97.4 | 96.3 | 98.8 | 97.1 | 95.6 | 98.8 | 96.7 | 94.9 |
| % LMW | 0.1 | 0.5 | 0.8 | 0.1 | 0.5 | 0.9 | 0.1 | 0.6 | 1.1 |

cIEF

| | | | | | | | | | |
|---|---|---|---|---|---|---|---|---|---|
| % Acidic | 22.162 | 22.456 | 23.907 | 22.169 | 25.026 | 26.032 | 22.573 | 26.621 | 28.835 |
| % Main 1 | 38.877 | 33.294 | 29.372 | 38.835 | 33.435 | 29.029 | 38.999 | 31.168 | 36.971 |
| % Main 2 | 30.866 | 29.083 | 28.956 | 31.198 | 31.578 | 30.237 | 31.295 | 31.235 | 31.792 |
| % Basic | 8.096 | 15.168 | 17.764 | 7.799 | 9.961 | 14.702 | 7.133 | 10.975 | 12.401 |

CE-SDS (NR)

| | | | | | | | | | |
|---|---|---|---|---|---|---|---|---|---|
| % Main IgG | NT | NT | 95.222 | NT | NT | 94.990 | NT | NT | 94.443 |
| % LMW | NT | NT | 4.453 | NT | NT | 4.634 | NT | NT | 5.164 |

CE-SDS (R)

| | | | | | | | | | |
|---|---|---|---|---|---|---|---|---|---|
| % Total | NT | NT | 93.311 | NT | NT | 93.456 | NT | NT | 91.462 |
| Purity | | | | | | | | | |
| % Impurity | NT | NT | 6.689 | NT | NT | 6.544 | NT | NT | 8.538 |

MFI

| | | | | | | | | | |
|---|---|---|---|---|---|---|---|---|---|
| ≥10 μm/mL | 39 | 13.77 | 13.77 | 6.88 | 13.80 | 22.96 | 0 | 2.3 | 9.18 |
| ≥25 μm/mL | 2.29 | 4.59 | 2.29 | 0 | 2.3 | 2.3 | 0 | 0 | 0 |

TABLE 10
Sotatercept Liquid Formulation Feasibility Stability (10 mM Acetate)

| | 50 mg/mL Sotatercept in 10 mM Acetate, 8% (weight/volume) Sucrose, pH 4.5-5.0 | | | | | |
|---|---|---|---|---|---|---|
| | pH 4.5 | | | 5.0 | | |
| | | 40° C. | | | 40° C. | |
| Assay | Initial | 1 M | 2 M | Initial | 1 M | 2 M |
| Conc. | 49.11 | 46.44 | | 46.54 | 46.53 | |
| (mg/mL) | | | | | | |
| pH | 4.99 | 5.22 | 5.35 | 5.2 | 5.54 | 5.42 |
| Turbidity | 0.140 | 0.152 | 0.170 | 0.138 | 0.148 | 0.162 |
| A₃₅₀₋₅₀₀ | | | | | | |

UP-SEC

| | | | | | | |
|---|---|---|---|---|---|---|
| % HMW | 0.7 | 1.8 | 2.6 | 0.6 | 1.6 | 2.1 |
| % Monomer | 98.8 | 97.1 | 95.7 | 98.8 | 97.4 | 96.1 |
| % LMW | 0.1 | 0.8 | 1.3 | 0.1 | 0.7 | 1.2 |

cIEF

| | | | | | | |
|---|---|---|---|---|---|---|
| % Acidic | 21.973 | 1.313 | 10.281 | 21.408 | 14.768 | 12.455 |
| % Main 1 | 30.705 | 28.696 | 24.651 | 39.580 | 29.774 | 26.364 |
| % Main 2 | 39.136 | 20.926 | 17.676 | 31.233 | 22.475 | 19.721 |
| % Basic | 8.186 | 37.064 | 47.392 | 7.779 | 32.983 | 41.46 |

CE-SDS (NR)

| | | | | | | |
|---|---|---|---|---|---|---|
| % Main IgG | NT | NT | NT | NT | NT | NT |
| % LMW | NT | NT | NT | NT | NT | NT |

CE-SDS (R)

| | | | | | | |
|---|---|---|---|---|---|---|
| % Total Purity | NT | NT | NT | NT | NT | NT |
| % Impurity | NT | NT | NT | NT | NT | NT |

MFI

| | | | | | | |
|---|---|---|---|---|---|---|
| ≥10 μm | 6.88 | 108.01 | 20.66 | 4.59 | 71.16 | 11.48 |
| ≥25 μm | 0 | 2.3 | 2.3 | 0 | 16.07 | 0 |

TABLE 11
Sotatercept Liquid Formulation Feasibility Stability (10 mM Acetate)

| | 50 mg/mL Sotatercept in 10 mM Acetate, 8% (weight/volume) Sucrose, pH 5.5-6.0 | | | | | |
|---|---|---|---|---|---|---|
| | pH 5.5 | | | 6.0 | | |
| | | 40° C. | | | 40° C. | |
| Assay | Initial | 1 M | 2 M | Initial | 1 M | 2 M |
| Conc. | 46.38 | 47.27 | | 46.75 | 47.76 | |
| (mg/mL) | | | | | | |
| pH | 5.65 | 5.88 | 5.87 | 5.83 | 6.06 | 6.11 |
| Turbidity | 0.125 | 0.138 | 0.144 | 0.131 | 0.143 | 0.142 |
| A₃₅₀₋₅₀₀ | | | | | | |

UP-SEC

| | | | | | | |
|---|---|---|---|---|---|---|
| % HMW | 0.6 | 1.1 | 1.4 | 0.6 | 1.1 | 1.3 |
| % Monomer | 98.8 | 97.5 | 96.5 | 98.8 | 97.5 | 96.4 |
| % LMW | 0.1 | 0.5 | 0.8 | 0.1 | 0.5 | 0.8 |

cIEF

| | | | | | | |
|---|---|---|---|---|---|---|
| % Acidic | 22.335 | 19.428 | 12.455 | 22.071 | 21.524 | 19.201 |
| % Main 1 | 38.859 | 32.583 | 26.364 | 38.083 | 28.718 | 29.914 |
| % Main 2 | 30.964 | 27.144 | 19.721 | 30.880 | 13.701 | 26.851 |
| % Basic | 7.841 | 20.845 | 41.460 | 8.967 | 16.557 | 24.460 |

CE-SDS (NR)

| | | | | | | |
|---|---|---|---|---|---|---|
| % Main IgG | NT | NT | NT | NT | NT | NT |
| % LMW | NT | NT | NT | NT | NT | NT |

CE-SDS (R)

| | | | | | | |
|---|---|---|---|---|---|---|
| % Total Purity | NT | NT | NT | NT | NT | NT |
| % Impurity | NT | NT | NT | NT | NT | NT |

MFI

| | | | | | | |
|---|---|---|---|---|---|---|
| ≥10 μm | 16.05 | 9.18 | 4.59 | 9.17 | 6.88 | 323.85 |
| ≥25 μm | 0 | 2.3 | 2.29 | 0 | 0 | 43.64 |

TABLE 12
Sotatercept Liquid Formulation Feasibility Stability (10 mM Succinate)

| | 50 mg/mL Sotatercept in 10 mM Succinate, 8% (weight/volume) Sucrose, pH 4.5-5.0 | | | | | |
|---|---|---|---|---|---|---|
| | pH 4.5 | | | 5.0 | | |
| | | 40° C. | | | 40° C. | |
| Assay | Initial | 1 M | 2 M | Initial | 1 M | 2 M |
| Conc. | 46.82 | 46.50 | | 46.64 | 47.07 | |
| (mg/mL) | | | | | | |
| pH | 4.78 | 4.81 | 4.93 | 5.07 | 5.37 | 5.24 |
| Turbidity | 0.156 | 0.170 | 0.197 | 0.144 | 0.155 | 0.169 |
| A₃₅₀₋₅₀₀ | | | | | | |

UP-SEC

| | | | | | | |
|---|---|---|---|---|---|---|
| % HMW | 0.9 | 2.7 | 4.3 | 0.7 | 1.7 | 2.5 |
| % Monomer | 98.5 | 95.6 | 93.2 | 98.8 | 97.1 | 95.7 |
| % LMW | 0.1 | 1.4 | 2.2 | 0.1 | 1.0 | 1.8 |

cIEF

| | | | | | | |
|---|---|---|---|---|---|---|
| % Acidic | 21.744 | 10.27 | 8.657 | 22.063 | 13.924 | 11.501 |
| % Main 1 | 38.603 | 22.619 | 18.535 | 38.475 | 27.277 | 23.057 |
| % Main 2 | 30.973 | 16.061 | 14.024 | 30.983 | 20.148 | 17.682 |
| % Basic | 8.68 | 51.050 | 58.785 | 8.48 | 38.651 | 47.761 |

CE-SDS (NR)

| | | | | | | |
|---|---|---|---|---|---|---|
| % Main IgG | NT | NT | 87.101 | NT | NT | 91.657 |
| % LMW | NT | NT | 11.070 | NT | NT | 7.199 |

CE-SDS (R)

| | | | | | | |
|---|---|---|---|---|---|---|
| % Total Purity | NT | NT | 72.618 | NT | NT | 82.578 |
| % Impurity | NT | NT | 27.382 | NT | NT | 17.428 |

MFI

| | | | | | | |
|---|---|---|---|---|---|---|
| ≥10 μm | 27.53 | 78.09 | 6.88 | 9.17 | 66.57 | 78.09 |
| ≥25 μm | 2.29 | 11.48 | 0 | 0 | 2.3 | 11.48 |

TABLE 13
Sotatercept Liquid Formulation Feasibility Stability (10 mM Glutamate)

| | 50 mg/mL Sotatercept in 10 mM Glutamate, 8% (weight/volume) Sucrose, pH 4.5-5.0 | | | | | |
|---|---|---|---|---|---|---|
| | pH 4.5 | | | 5.0 | | |
| | | 40° C. | | | 40° C. | |
| Assay | Initial | 1 M | 2 M | Initial | 1 M | 2 M |
| Conc. | 46.65 | 46.85 | 46.93 | 47.55 | 47.18 | 47.40 |
| (mg/mL) | | | | | | |
| pH | 4.99 | 5.11 | 5.27 | 5.47 | 5.8 | 5.68 |
| Turbidity | 0.149 | 0.167 | 0.195 | 0.138 | 0.150 | 0.162 |
| A₃₅₀₋₅₀₀ | | | | | | |

UP-SEC

| | | | | | | |
|---|---|---|---|---|---|---|
| % HMW | 0.7 | 1.9 | 2.8 | 0.6 | 1.3 | 1.7 |
| % Monomer | 98.7 | 97.1 | 95.2 | 98.8 | 97.5 | 96.1 |
| % LMW | 0.1 | 1.0 | 1.9 | 0.1 | 0.6 | 1.1 |

cIEF

| | | | | | | |
|---|---|---|---|---|---|---|
| % Acidic | 22.272 | 13.005 | 10.482 | 22.681 | 18.419 | 16.988 |
| % Main 1 | 38.186 | 27.182 | 22.944 | 38.409 | 31.751 | 28.646 |
| % Main 2 | 31.174 | 20.128 | 16.706 | 31.259 | 26.359 | 24.429 |
| % Basic | 8.368 | 39.684 | 49.868 | 7.652 | 23.470 | 29.937 |

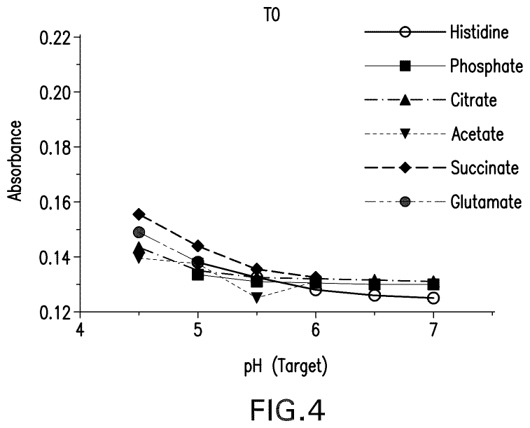

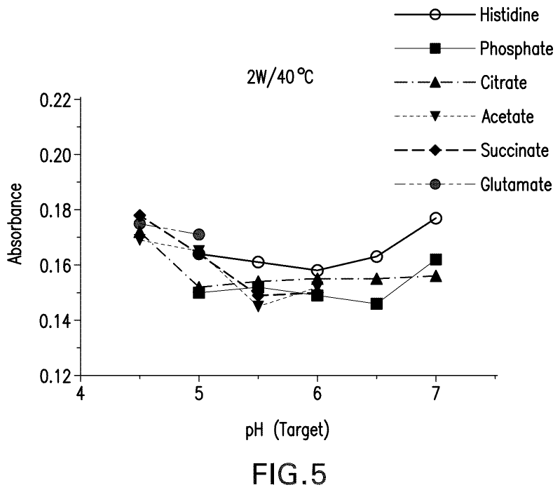

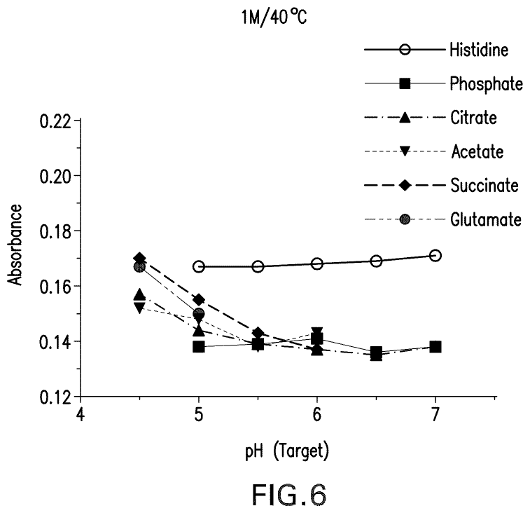

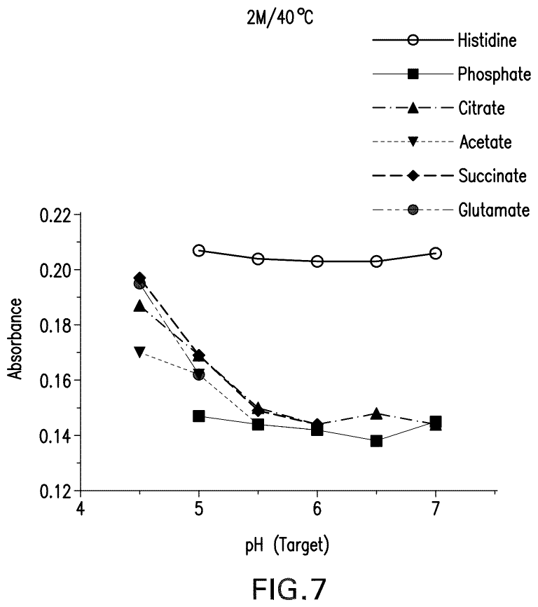

As shown in the Tables 4-13 above and in FIG. 4-7, all the formulations exhibit reasonable stability at elevated temperatures suggesting all the buffer systems tested are feasible for liquid formulation. Out of these, histidine was least stable, and the other buffers behaved equally.

The behavior of sotatercept was also analyzed in 6-buffers at a pH of 3, 5.8 and 8 and 8% sucrose. The buffers tested were citrate, histidine, succinate, lactate, MES and TRIS. The results are shown in Tables 14-30. The formulations were tested for biophysical stability (turbidity), high molecular weight formation (via UPSEC), aggregation (via UPSEC) and fragmentation (via R-CESDS and NR-CESDSUPSEC) and charge distribution (via cIEF) at time 0 (TO), 15 days and 30 days.

TABLE 14
Sotatercept Liquid Formulation Feasibility Stability (5 mM Citrate)

| | 50 mg/mL Sotatercept in 5 mM Citrate, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 51.3 | 57.5 | 66.7 | 54.1 | 61.4 | 63.3 | 53.9 | 60.2 | 63.3 |
| (mg/mL) | | | | | | | | | |
| pH | 3.54 | 3.74 | NT | 5.73 | 5.74 | 5.74 | 7.09 | 7.03 | 6.27 |
| Turbidity | 0.140 | 0.408 | NT | 0.148 | 0.193 | 0.224 | 0.154 | 0.198 | 0.240 |
| A₃₅₀₋₅₀₀ | | | | | | | | | |

UP-SEC

| | | | | | | | | | |
|---|---|---|---|---|---|---|---|---|---|
| % HMW | 1.6 | 83.6 | NT | 0.9 | 1.7 | NT | 1.2 | 1.8 | NT |
| % Monomer | 95.6 | 11.8 | NT | 96.4 | 94.1 | NT | 95.9 | 93.1 | NT |
| % LMW | 2.7 | 4.6 | NT | 2.7 | 4.2 | NT | 2.8 | 5.1 | NT |

TABLE 15
Sotatercept Liquid Formulation Feasibility Stability (10 mM Citrate)

| | 50 mg/mL Sotatercept in 10 mM Citrate, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 54.4 | 57.6 | NT | 53.7 | 62.8 | 62.8 | 53.7 | 62.5 | 62.8 |
| (mg/mL) | | | | | | | | | |
| pH | 3.33 | 3.57 | NT | 5.8 | 5.75 | 5.15 | 7.28 | 7.13 | 6.99 |
| Turbidity | 0.149 | 0.455 | NT | 0.147 | 0.196 | 0.241 | 0.160 | 0.219 | 0.271 |
| A₃₅₀₋₅₀₀ | | | | | | | | | |

UP-SEC

| | | | | | | | | | |
|---|---|---|---|---|---|---|---|---|---|
| % HMW | 2.4 | 93.9 | NT | 0.9 | 1.6 | NT | 1.3 | 2.0 | NT |
| % Monomer | 94.6 | 1.8 | NT | 96.5 | 94.4 | NT | 96.0 | 92.6 | NT |
| % LMW | 2.9 | 4.3 | NT | 2.6 | 4.0 | NT | 2.6 | 5.4 | NT |

TABLE 16
Sotatercept Liquid Formulation Feasibility Stability (50 mM Citrate)

| | 50 mg/mL Sotatercept in 50 mM Citrate, 8% (weight/volume) Sucrose, pH 5.8-8.0 | | | | | |
|---|---|---|---|---|---|---|
| | pH 5.8 | | | pH 8.0 | | |
| | | 50° C. | | | 50° C. | |
| Assay | Initial | 2 W | 1 M | Initial | 2 W | 1 M |
| Conc. | 47.9 | 53.2 | 55.7 | 51.7 | 53.8 | 66.7 |
| (mg/mL) | | | | | | |
| pH | 5.93 | 5.96 | NT | 7.16 | 7.16 | 5.85 |
| Turbidity | 0.141 | 0.179 | 0.248 | 0.173 | 0.211 | NT |
| A₃₅₀₋₅₀₀ | | | | | | |

UP-SEC

| | | | | | | |
|---|---|---|---|---|---|---|
| % HMW | 0.8 | 1.4 | NT | 1.3 | 2.2 | NT |
| % Monomer | 96.5 | 94.5 | NT | 95.9 | 90.8 | NT |
| % LMW | 2.6 | 4.1 | NT | 2.8 | 7.0 | NT |

TABLE 17
Sotatercept Liquid Formulation Feasibility Stability (5 mM Succinate)

| | 50 mg/mL Sotatercept in 5 mM Succinate, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 53.0 | 57.5 | 60.3 | 53.2 | 59.8 | 61.4 | 53.6 | 60.3 | 62.6 |
| (mg/mL) | | | | | | | | | |
| pH | 4.11 | 4.22 | NT | 5.71 | 5.75 | 2.52 | 6.82 | 6.82 | 5.54 |
| Turbidity | 0.155 | 0.282 | 0.707 | 0.145 | 0.193 | 0.246 | 0.148 | 0.198 | 0.241 |
| A₃₅₀₋₅₀₀ | | | | | | | | | |

UP-SEC

| | | | | | | | | | |
|---|---|---|---|---|---|---|---|---|---|
| % HMW | 1.8 | 42.7 | NT | 0.9 | 2.1 | NT | 1.2 | 1.7 | NT |
| % Monomer | 95.5 | 50.8 | NT | 96.6 | 93.7 | NT | 96.2 | 93.4 | NT |
| % LMW | 2.7 | 6.5 | NT | 2.5 | 4.1 | NT | 2.6 | 4.9 | NT |

TABLE 18
Sotatercept Liquid Formulation Feasibility Stability (10 mM Succinate)

| | 50 mg/mL Sotatercept in 10 mM Succinante, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 49.8 | 57.8 | 59.0 | 54.0 | 59.2 | 64.8 | 53.1 | 57.0 | 62.8 |
| (mg/mL) | | | | | | | | | |
| pH | 2.92 | 4.04 | 5.83 | 5.82 | 5.84 | 4.1 | 6.96 | 6.94 | 5.78 |
| Turbidity | 0.140 | 0.320 | 0.786 | 0.152 | 0.192 | 0.254 | 0.155 | 0.193 | 0.257 |
| A₃₅₀₋₅₀₀ | | | | | | | | | |

UP-SEC

| | | | | | | | | | |
|---|---|---|---|---|---|---|---|---|---|
| % HMW | 1.6 | 48.7 | NT | 0.9 | 1.8 | NT | 1.2 | 1.7 | NT |
| % Monomer | 95.6 | 45.5 | NT | 96.6 | 94.0 | NT | 96.3 | 93.5 | NT |
| % LMW | 2.8 | 5.8 | NT | 2.5 | 4.2 | NT | 2.5 | 4.8 | NT |

TABLE 19
Sotatercept Liquid Formulation Feasibility Stability (50 mM Succinante)

| | 50 mg/mL Sotatercept in 50 mM Succinante, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 55.8 | 55.2 | NT | 54.3 | 56.7 | 46.8 | 54.4 | 59.6 | 63.1 |
| (mg/mL) | | | | | | | | | |
| pH | 3.49 | 3.64 | 6.53 | 5.77 | 5.78 | NT | 7.31 | 7.19 | 5.21 |
| Turbidity | 0.151 | 0.530 | NT | 0.155 | 0.214 | 0.331 | 0.168 | 0.248 | 0.382 |
| A₃₅₀₋₅₀₀ | | | | | | | | | |

UP-SEC

| | | | | | | | | | |
|---|---|---|---|---|---|---|---|---|---|
| % HMW | 1.7 | 77.5 | NT | 0.9 | 1.7 | NT | 1.4 | 2.4 | NT |
| % Monomer | 95.4 | 17.3 | NT | 96.6 | 94.2 | NT | 96.2 | 90.8 | NT |
| % LMW | 2.8 | 5.2 | NT | 2.5 | 4.1 | NT | 2.4 | 6.8 | NT |

TABLE 20
Sotatercept Liquid Formulation Feasibility Stability (5 mM Histidine)

| | 50 mg/mL Sotatercept in 5 mM Histidine, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 52.4 | 58.7 | 61.3 | 54.9 | 57.7 | 65.0 | 54.2 | 58.2 | NT |
| (mg/mL) | | | | | | | | | |
| pH | 4.46 | 4.64 | 6.73 | 5.36 | 5.44 | 3.29 | 6.61 | 6.62 | 4.19 |
| Turbidity | 0.192 | 0.311 | 0.535 | 0.154 | 0.237 | 0.405 | 0.152 | .0220 | NT |
| A₃₅₀₋₅₀₀ | | | | | | | | | |

UP-SEC

| | | | | | | | | | |
|---|---|---|---|---|---|---|---|---|---|
| % HMW | 1.8 | 36.3 | NT | 0.9 | 8.4 | NT | 1.8 | 1.1 | NT |
| % Monomer | 95.7 | 57.9 | NT | 96.5 | 87.2 | NT | 95.6 | 94.2 | NT |
| % LMW | 2.6 | 5.8 | NT | 2.6 | 4.4 | NT | 2.6 | 4.6 | NT |

TABLE 21
Sotatercept Liquid Formulation Feasibility Stability (10 mM Histidine)

| | 50 mg/mL Sotatercept in 10 mM Histidine, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 52.7 | 55.3 | 63.5 | 51.8 | 56.9 | 72.4 | 53.9 | 58.8 | 62.5 |
| (mg/mL) | | | | | | | | | |
| pH | 4.17 | 4.33 | NT | 5.43 | 5.51 | NT | 6.61 | 6.64 | NT |
| Turbidity | 0.166 | 0.298 | NT | 0.149 | 0.243 | NT | 0.150 | 0.269 | 0.411 |
| A₃₅₀₋₅₀₀ | | | | | | | | | |

UP-SEC

| | | | | | | | | | |
|---|---|---|---|---|---|---|---|---|---|
| % HMW | 0.8 | 50.5 | NT | 0.8 | 6.8 | NT | 0.8 | 1.3 | NT |
| % Monomer | 96.7 | 47 | NT | 96.5 | 87.3 | NT | 96.7 | 93.7 | NT |
| % LMW | 2.5 | 2.4 | NT | 2.6 | 5.8 | NT | 2.4 | 5.0 | NT |

TABLE 22
Sotatercept Liquid Formulation Feasibility Stability (50 mM Histidine)

| | 50 mg/mL Sotatercept in 50 mM Histidine, 8% (weight/volume) Sucrose, pH 3.0-8.0 | | | | | | | | |
|---|---|---|---|---|---|---|---|---|---|
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 52.5 | 59.6 | 61.5 | 53.8 | 58.2 | 61.5 | 54.7 | 57.8 | 63.6 |
| (mg/mL) | | | | | | | | | |
| pH | 5.97 | 4.72 | 5.45 | 5.63 | 5.85 | 3.93 | 5.71 | 7.06 | 4.61 |
| Turbidity | 0.167 | 0.419 | 0.833 | 0.157 | 0.302 | 0.539 | 0.155 | 0.301 | 0.535 |
| A₃₅₀₋₅₀₀ | | | | | | | | | |

UP-SEC

| | | | | | | | | | |
|---|---|---|---|---|---|---|---|---|---|
| % HMW | 1.4 | 38.4 | NT | 0.7 | 2.8 | NT | 0.8 | 0.9 | NT |
| % Monomer | 96.2 | 56.0 | NT | 96.9 | 92.8 | NT | 96.7 | 93.5 | NT |
| % LMW | 2.4 | 5.5 | NT | 2.4 | 4.4 | NT | 2.5 | 5.6 | NT |

TABLE 23
Sotatercept Liquid Formulation Feasibility Stability (10 mM Lactate)

| | 50 mg/mL Sotatercept in 10 mM Lactate, 8% (weight/volume) Sucrose, pH 5.8-8.0 | | | | | |
|---|---|---|---|---|---|---|
| | pH 5.8 | | | pH 8.0 | | |
| | | 50° C. | | | 50° C. | |
| Assay | Initial | 2 W | 1 M | Initial | 2 W | 1 M |
| Conc. | 51.4 | 60 | 59.5 | 53.5 | 57.5 | 62.3 |
| (mg/mL) | | | | | | |
| pH | 8.05 | 5.87 | 5.67 | 5.93 | 5.95 | 5.71 |
| Turbidity | 0.143 | 0.179 | 0.235 | 0.147 | 0.182 | 0.249 |
| A₃₅₀₋₅₀₀ | | | | | | |

UP-SEC

| | | | | | | |
|---|---|---|---|---|---|---|
| % HMW | 0.9 | 2 | NT | 0.9 | 1.8 | NT |
| % Monomer | 96.7 | 93.2 | NT | 96.8 | 93.5 | NT |
| % LMW | 2.4 | 4.8 | NT | 2.4 | 4.7 | NT |

TABLE 24
Sotatercept Liquid Formulation Feasibility
Stability (50 mM Lactate)

| | 50 mg/mL Sotatercept in 50 mM Lactate, | | | | | |
|---|---|---|---|---|---|---|
| | 8% (weight/volume) Sucrose, pH 5.8-8.0 | | | | | |
| | pH 5.8 | | | pH 8.0 | | |
| | | 50° C. | | | 50° C. | |
| Assay | Initial | 2 W | 1 M | Initial | 2 W | 1 M |
| Conc. (mg/mL) | 54 | 58.7 | 62.3 | 54.8 | 64 | 65.4 |
| pH | 7.65 | 5.83 | 4.78 | 5.95 | 6.06 | 5.76 |
| Turbidity | 0.155 | 0.197 | 0.266 | 0.162 | 0.205 | 0.235 |
| A₃₅₀₋₅₀₀ | | | | | | |

UP-SEC

| | | | | | | |
|---|---|---|---|---|---|---|
| % HMW | 0.9 | 1.8 | NT | 0.9 | 1.7 | NT |
| % Monomer | 96.7 | 94.1 | NT | 96.7 | 93.5 | NT |
| % LMW | 2.5 | 4.1 | NT | 2.4 | 4.8 | NT |

TABLE 25
Sotatercept Liquid Formulation Feasibility Stability (5 mM MES)

| | 50 mg/mL Sotatercept in 5 mM MES, | | | | | |
|---|---|---|---|---|---|---|
| | 8% (weight/volume) Sucrose, pH 5.8-8.0 | | | | | |
| | pH 5.8 | | | pH 8.0 | | |
| | | 50° C. | | | 50° C. | |
| Assay | Initial | 2 W | 1 M | Initial | 2 W | 1 M |
| Conc. (mg/mL) | 54.4 | 58.5 | 63.1 | 54.1 | 58.1 | 62.8 |
| pH | 5.59 | 5.66 | 5.11 | 7.34 | 6.72 | 4.02 |
| Turbidity | 0.153 | 0.196 | 0.283 | 0.150 | 0.184 | 0.215 |
| A₃₅₀₋₅₀₀ | | | | | | |

UP-SEC

| | | | | | | |
|---|---|---|---|---|---|---|
| % HMW | 0.9 | 3.3 | NT | 1.0 | 1.5 | NT |
| % Monomer | 96.6 | 92.5 | NT | 96.4 | 94.1 | NT |
| % LMW | 2.5 | 4.2 | NT | 2.6 | 4.4 | NT |

TABLE 26
Sotatercept Liquid Formulation Feasibility Stability (10 mM MES)

| | 50 mg/mL Sotatercept in 10 mM MES, | | | | | |
|---|---|---|---|---|---|---|
| | 8% (weight/volume) Sucrose, pH 5.8-8.0 | | | | | |
| | pH 5.8 | | | pH 8.0 | | |
| | | 50° C. | | | 50° C. | |
| Assay | Initial | 2 W | 1 M | Initial | 2 W | 1 M |
| Conc. (mg/mL) | 53.7 | 57.3 | 62.3 | 53.9 | 58.0 | 64.2 |
| pH | 5.89 | 5.64 | 7.01 | 5.73 | 7.06 | 5.5 |
| Turbidity | 0.150 | 0.194 | 0.273 | 0.153 | 0.190 | 0.273 |
| A₃₅₀₋₅₀₀ | | | | | | |

UP-SEC

| | | | | | | |
|---|---|---|---|---|---|---|
| % HMW | 0.9 | 3.5 | NT | 1.1 | 1.6 | NT |
| % Monomer | 96.6 | 92.6 | NT | 96.5 | 93.2 | NT |
| % LMW | 2.5 | 4.0 | NT | 2.4 | 5.1 | NT |

TABLE 27
Sotatercept Liquid Formulation Feasibility Stability (50 mM MES)

| | 50 mg/mL Sotatercept in 50 mM MES, | | | | | |
|---|---|---|---|---|---|---|
| | 8% (weight/volume) Sucrose, pH 5.8-8.0 | | | | | |
| | pH 5.8 | | | pH 8.0 | | |
| | | 50° C. | | | 50° C. | |
| Assay | Initial | 2 W | 1 M | Initial | 2 W | 1 M |
| Conc. (mg/mL) | 53.5 | 58.5 | 63.2 | 54.9 | 63.9 | 63.9 |
| pH | 7.57 | 5.88 | 7.01 | 5.86 | 7.59 | 4.69 |
| Turbidity | 0.154 | 0.223 | 0.297 | 0.166 | 0.239 | 0.260 |
| A₃₅₀₋₅₀₀ | | | | | | |

UP-SEC

| | | | | | | |
|---|---|---|---|---|---|---|
| % HMW | 0.9 | 2.2 | NT | 1.2 | 2.4 | NT |
| % Monomer | 96.6 | 93.5 | NT | 96.2 | 91.4 | NT |
| % LMW | 2.5 | 4.3 | NT | 2.6 | 6.2 | NT |

TABLE 28
Sotatercept Liquid Formulation Feasibility Stability (5 mM Tris)

| | 50 mg/mL Sotatercept in 5 mM Tris, | | | | | | | | |
|---|---|---|---|---|---|---|---|---|---|
| | 8% (weight/volume) Sucrose, pH 3.0-8.0 | | | | | | | | |
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 55.2 | 61.1 | 64.7 | 54.3 | 58.8 | 63.5 | 54.2 | 59.5 | 64.2 |
| (mg/mL) | | | | | | | | | |
| pH | 6.98 | 5.63 | 6.88 | 5.60 | 5.94 | 4.93 | 6.57 | 7.34 | 5.78 |
| Turbidity | 0.154 | 0.201 | 0.261 | 0.150 | 0.188 | 0.232 | 0.156 | 0.193 | 0.239 |
| A₃₅₀₋₅₀₀ | | | | | | | | | |

UP-SEC

| | | | | | | | | | |
|---|---|---|---|---|---|---|---|---|---|
| % HMW | 0.9 | 2.9 | NT | 0.9 | 2.0 | NT | 1.0 | 1.4 | NT |
| % Monomer | 96.6 | 92.7 | NT | 06.6 | 93.8 | NT | 96.5 | 93.2 | NT |
| % LMW | 2.5 | 4.4 | NT | 2.5 | 4.2 | NT | 2.5 | 5.4 | NT |

TABLE 29
Sotatercept Liquid Formulation Feasibility Stability (10 mM Tris)

| | 50 mg/mL Sotatercept in 10 mM Tris, | | | | | | | | |
|---|---|---|---|---|---|---|---|---|---|
| | 8% (weight/volume) Sucrose, pH 3.0-8.0 | | | | | | | | |
| | pH 3.0 | | | pH 5.8 | | | pH 8.0 | | |
| | | 50° C. | | | 50° C. | | | 50° C. | |
| Assay | Initial | 2 W | 1 M | Initial | 2 W | 1 M | Initial | 2 W | 1 M |
| Conc. | 54.2 | 59.6 | 64.9 | 53.9 | 60.4 | 63.6 | 55.6 | 65.0 | 65.4 |
| (mg/mL) | | | | | | | | | |
| pH | 5.66 | 5.62 | 5.80 | 5.93 | 5.87 | 4.87 | 5.86 | 7.64 | 5.83 |
| Turbidity | 0.159 | 0.200 | 0.262 | 0.153 | 0.194 | 0.258 | 0.158 | 0.220 | 0.254 |
| A₃₅₀₋₅₀₀ | | | | | | | | | |

UP-SEC

| | | | | | | | | | |
|---|---|---|---|---|---|---|---|---|---|
| % HMW | 0.9 | 2.9 | NT | 0.9 | 2.0 | NT | 1.0 | 1.5 | NT |
| % Monomer | 96.7 | 93.0 | NT | 96.8 | 94.1 | NT | 96.5 | 92.2 | NT |
| % LMW | 2.4 | 4.2 | NT | 2.4 | 3.8 | NT | 2.5 | 6.3 | NT |

TABLE 30
Sotatercept Liquid Formulation Feasibility Stability (50 mM Tris)

| | 50 mg/mL Sotatercept in 50 mM Tris, | | | | | |
|---|---|---|---|---|---|---|
| | 8% (weight/volume) Sucrose, pH 5.8-8.0 | | | | | |
| | pH 5.8 | | | pH 8.0 | | |
| | | 50° C. | | | 50° C. | |
| Assay | Initial | 2 W | 1 M | Initial | 2 W | 1 M |
| Conc. (mg/mL) | 55.8 | 61.9 | 65.0 | 54.4 | 60.7 | 67.0 |
| pH | 5.94 | 5.91 | 6.02 | 5.91 | 8.06 | 5.48 |
| Turbidity | 0.160 | 0.201 | 0.243 | 0.166 | 0.229 | 0.348 |
| A₃₅₀₋₅₀₀ | | | | | | |

UP-SEC

| | | | | | | |
|---|---|---|---|---|---|---|
| % HMW | 0.8 | 1.7 | NT | 0.9 | 1.6 | NT |
| % Monomer | 96.7 | 94.0 | NT | 96.7 | 90.8 | NT |
| % LMW | 2.5 | 4.3 | NT | 2.4 | 7.7 | NT |

Based on the data presented in Tables 14-30, the buffers tested had comparable stability at pH 5.8 and pH 8.0 across buffer strengths. At lower pH, however, there was significant decrease in stability for all buffers tested. While all buffers were comparable citrate and succinate marginally performed belier than others.

### Effect of Metal Chelator on Sotatercept

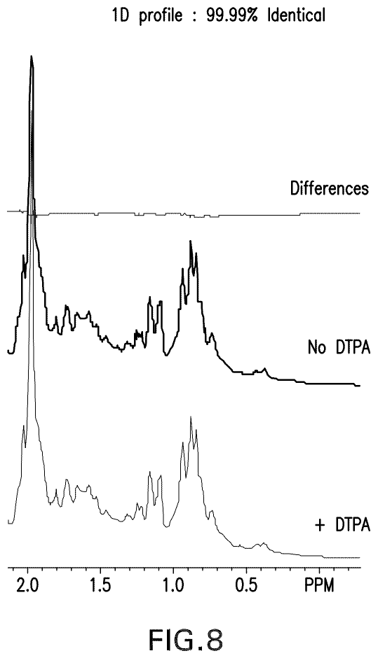

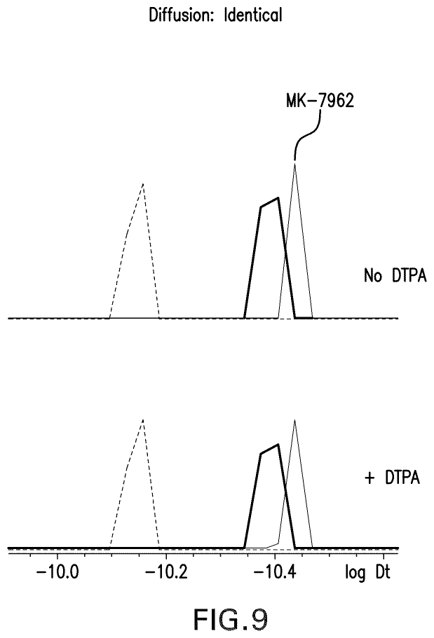

The effect of metal chelator, DTPA, on sotatercept (SEQ ID NO: 32) formulated in citrate buffer, pH 5.8 were studied by TH NMR profiles, 2D NMR fingerprints & NMR measurements of protein translational diffusion. The results of the TD profiles and protein diffusion are shown in FIGS. 8 and 9. Identical behavior was observed in protein TH NMR spectra of sotatercept with and without DTPA (FIG. 8) indicating no direct effect from binding or other interactions could be detected by this technique. Additionally, data from protein-detected diffusion measurements with and without DTPA (FIG. 9), showed no differences in behavior, indicating that DTPA addition has no effect on sotatercept behavior.

### Colloidal Stability and Surfactant Screen

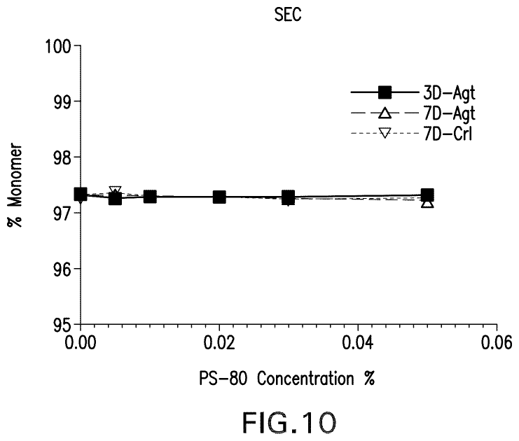

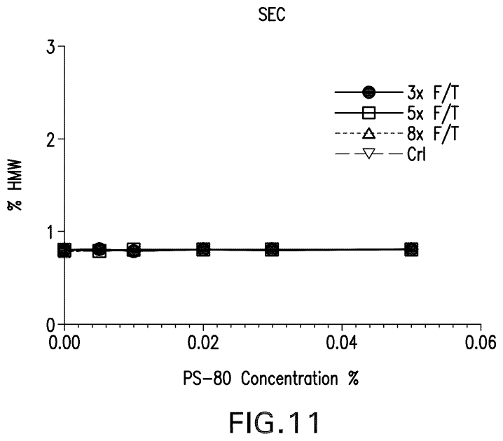

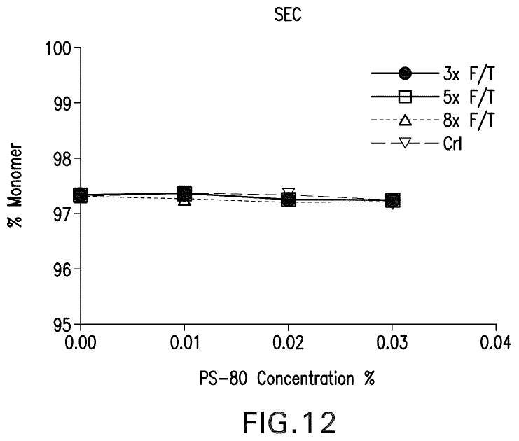

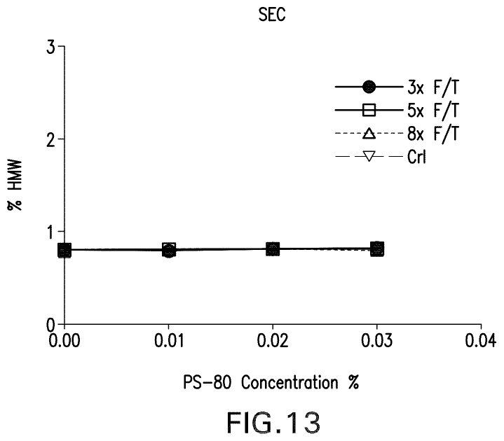

Colloidal stability studies for sotatercept (SEQ ID NO:32) were carried out by agitation stress by shaking the vials containing sotatercept in 10 mM citrate buffer, 8% sucrose with varying levels of PS80 and by freeze-thaw cycling (one cycle-freeze the vial at −80° C. and thaw at room temperature). The results are shown in FIG. 10-13. FIG. 10 shows percent of high molecular weight species (% HMW) detected in vials containing sotatercept in 10 mM citrate buffer, 8% sucrose with 0%, 0.005%, 0.01%, 0.02%, 0.03% and 0.05% of PS80, agitated for 3 and 7 days. FIG. 11 shows % HMW detected in vials containing sotatercept in 10 mM citrate buffer, 8% sucrose with 0%, 0.005%, 0.01%, 0.02%, 0.03% and 0.05% of PS80, that have undergone three, five and eight freeze-thaw cycles. FIGS. 12 and 13 shows % Monomer (FIG. 12) and % HMW (FIG. 13) detected in vials containing sotatercept in 10 mM citrate buffer, 8% sucrose with 0%, 0.01%, 0.02%, and 0.03% of PS80, that have undergone three, five and eight freeze-thaw cycles. No change on % HMW or subvisible particle formation was observed after seven days of agitation or 8 freeze/thaw cycles. Also, a head-to-head comparison of polysorbate 80 and poloxamer 188 was conducted, 1-Mo/25° C. The results show that molecule is stable against agitation stress and there is no difference between the be PS80 or P188 and they both behave similarly. All analytical attributes were comparable after 1-Mo stress at 25° C. in the prototype stability.

Results of additional surfactant stability studies are shown in Tables 31-35. Stability studies shown in Tables 31-35 were carried out by agitation stress by shaking vials containing sotatercept in 10 mM citrate buffer and 8% sucrose with varying levels of surfactants. The surfactants screened were polaxamer 188, sodium dodecyl sulfate (SDS), N-dodecyl-β-D-maltoside (DDM), polysorbate 20 and triton X at 0.02 mg/mL, 0.2 mg/mL and 2 mg/mL for 4 days and 7 days. Table 36 shows the results of the stability studies for the control sotatercept in 10 mM citrate buffer and 8% sucrose and no surfactant.

TABLE 31
Sotatercept Surfactant Screen (N-dodecyl-β-D-maltoside in Citrate)

| | 50 mg/mL Sotatercept in DDM, 10 mM Citrate, | | | | | | | | |
|---|---|---|---|---|---|---|---|---|---|
| | 8% (weight/volume) Sucrose, pH 5.8 | | | | | | | | |
| | 2 mg/mL DDM | | | 0.2 mg/mL DDM | | | 0.02 mg/mL DDM | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | NT | NT | NT | 52.62 | 55.51 | 56.68 | 54.53 | 56.64 | 52.81 |
| (mg/mL) | | | | | | | | | |
| pH | 5.23 | 6.01 | 4.58 | 5.10 | 5.80 | 4.50 | 5.23 | 5.78 | 5.38 |
| Turbidity | NT | NT | NT | 0.200 | 0.230 | 0.230 | 0.220 | 0.240 | 0.170 |
| A₃₅₀₋₅₀₀ | | | | | | | | | |

UP-SEC

| | | | | | | | | | |
|---|---|---|---|---|---|---|---|---|---|
| % HMW | 1.4 | 1.4 | 1.4 | 1.4 | 1.4 | 1.5 | 1.4 | 1.4 | 1.4 |
| % Monomer | 93.1 | 93.2 | 93.1 | 93.1 | 93.2 | 93.1 | 93.1 | 93.2 | 93.2 |
| % LMW | 5.5 | 5.4 | 5.5 | 5.4 | 5.3 | 5.4 | 5.5 | 5.3 | 5.4 |

cIEF

| | | | | | | | | | |
|---|---|---|---|---|---|---|---|---|---|
| % Acidic | 14.1 | 13.6 | 13.8 | 14.0 | 13.9 | 14.0 | 14.1 | 14.2 | 14.2 |
| % Main 1 | 46.0 | 46.7 | 46.6 | 45.7 | 46.1 | 46.4 | 45.8 | 46.2 | 46.3 |
| % Main 2 | 27.1 | 27.2 | 27.0 | 27.1 | 27.2 | 27.0 | 27.3 | 27.0 | 27.0 |
| % Basic | 12.8 | 12.5 | 12.6 | 13.2 | 12.8 | 12.5 | 12.8 | 12.6 | 12.5 |

TABLE 32
Sotatercept Surfactant Screen (Polaxamer-188 in Citrate)

| | 50 mg/mL Sotatercept in P-188, 10 mM Citrate, | | | | | | | | |
|---|---|---|---|---|---|---|---|---|---|
| | 8% (weight/volume) Sucrose, pH 5.8 | | | | | | | | |
| | 2 mg/mL P-188 | | | 0.2 mg/mL P-188 | | | 0.02 mg/mL P-188 | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | 55.48 | 54.63 | 52.65 | 56.25 | 53.13 | 53.90 | 52.90 | 55.60 | 56.51 |
| (mg/mL) | | | | | | | | | |
| pH | 5.69 | 5.94 | 4.16 | 5.32 | 6.15 | 4.72 | 5.00 | 6.31 | 5.65 |
| Turbidity | 0.200 | 0.260 | 0.215 | 0.215 | 0.235 | 0.205 | 0.200 | 0.235 | 0.175 |
| A₃₅₀₋₅₀₀ | | | | | | | | | |

UP-SEC

| | | | | | | | | | |
|---|---|---|---|---|---|---|---|---|---|
| % HMW | 1.4 | 1.4 | 1.4 | 1.6 | 1.4 | 1.4 | 1.4 | 1.4 | 1.4 |
| % Monomer | 93.1 | 93.2 | 93.1 | 93.0 | 93.3 | 93.1 | 93.2 | 93.3 | 93.2 |
| % LMW | 5.4 | 5.4 | 5.5 | 5.5 | 5.3 | 5.5 | 5.4 | 5.3 | 5.4 |

cIEF

| | | | | | | | | | |
|---|---|---|---|---|---|---|---|---|---|
| % Acidic | 13.9 | 13.9 | 13.8 | 13.9 | 14.4 | 14.4 | 14.0 | 14.5 | 14.4 |
| % Main 1 | 46.3 | 46.5 | 46.5 | 46.6 | 45.8 | 46.0 | 46.0 | 46.0 | 46.2 |
| % Main 2 | 27.1 | 27.1 | 26.9 | 27.0 | 26.7 | 26.5 | 27.3 | 26.9 | 27.0 |
| % Basic | 12.7 | 12.5 | 12.8 | 12.5 | 13.2 | 13.0 | 12.7 | 12.5 | 12.5 |

TABLE 33
Sotatercept Surfactant Screen (Polysorbate-20 in Citrate)

| | 50 mg/mL Sotatercept in PS-20, 10 mM Citrate, | | | | | | | | |
|---|---|---|---|---|---|---|---|---|---|
| | 8% (weight/volume) Sucrose, pH 5.8 | | | | | | | | |
| | 2 mg/mL PS-20 | | | 0.2 mg/mL PS-20 | | | 0.02 mg/mL PS-20 | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | 54.93 | 54.09 | 52.07 | 55.33 | 54.80 | 53.15 | 52.54 | 55.42 | 55.24 |
| (mg/mL) | | | | | | | | | |
| pH | 5.70 | 5.66 | 4.48 | 5.28 | 5.80 | 4.67 | 5.49 | 6.31 | 4.00 |
| Turbidity | 0.155 | 0.175 | 0.130 | 0.180 | 0.170 | 0.175 | 0.185 | 0.235 | 0.175 |
| A₃₅₀₋₅₀₀ | | | | | | | | | |

UP-SEC

| | | | | | | | | | |
|---|---|---|---|---|---|---|---|---|---|
| % HMW | 1.4 | 1.4 | 1.4 | 1.4 | 1.4 | 1.4 | 1.4 | 1.4 | 1.4 |
| % Monomer | 93.1 | 93.2 | 93.1 | 93.2 | 93.3 | 93.1 | 93.2 | 93.3 | 93.2 |
| % LMW | 5.4 | 5.4 | 5.5 | 5.4 | 5.3 | 5.5 | 5.4 | 5.3 | 5.4 |

cIEF

| | | | | | | | | | |
|---|---|---|---|---|---|---|---|---|---|
| % Acidic | 14.1 | 13.8 | 14.1 | 14.5 | 14.1 | 14.5 | 14.0 | 13.7 | 14.2 |
| % Main 1 | 46.2 | 46.6 | 46.1 | 46.0 | 46.1 | 46.0 | 45.9 | 46.9 | 45.9 |
| % Main 2 | 27.1 | 26.9 | 26.9 | 26.7 | 27.1 | 26.6 | 26.9 | 27.1 | 27.0 |
| % Basic | 12.6 | 12.6 | 12.9 | 12.8 | 12.7 | 12.8 | 13.2 | 12.3 | 13.0 |

TABLE 34
Sotatercept Surfactant Screen (Sodium Dodecyl Sulfate in Citrate)

| | 50 mg/mL Sotatercept in SDS, 10 mM Citrate, | | | | | | | | |
|---|---|---|---|---|---|---|---|---|---|
| | 8% (weight/volume) Sucrose, pH 5.8 | | | | | | | | |
| | 2 mg/mL SDS | | | 0.2 mg/mL SDS | | | 0.02 mg/mL SDS | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | 51.58 | 52.61 | 53.02 | 56.30 | 53.01 | 54.72 | 53.48 | 55.79 | 53.96 |
| (mg/mL) | | | | | | | | | |
| pH | 5.85 | NT | 4.88 | 5.63 | NT | 5.60 | 5.75 | 6.31 | 5.87 |
| Turbidity | 0.205 | 0.245 | 0.200 | 0.180 | 0.240 | 0.185 | 0.155 | 0.225 | 0.185 |
| A₃₅₀₋₅₀₀ | | | | | | | | | |

UP-SEC

| | | | | | | | | | |
|---|---|---|---|---|---|---|---|---|---|
| % HMW | 4.9 | 1.9 | 2.6 | 1.7 | 1.7 | 1.7 | 1.4 | 1.4 | 1.4 |
| % Monomer | 89.4 | 92.7 | 91.6 | 92.8 | 92.9 | 92.8 | 93.2 | 93.2 | 93.2 |
| % LMW | 5.7 | 5.4 | 5.7 | 5.5 | 5.4 | 5.5 | 5.4 | 5.4 | 5.4 |

cIEF

| | | | | | | | | | |
|---|---|---|---|---|---|---|---|---|---|
| % Acidic | 13.7 | 14.0 | 13.7 | 14.0 | 13.8 | 13.7 | 14.2 | 14.1 | 14.0 |
| % Main 1 | 45.6 | 46.5 | 47.1 | 46.1 | 46.8 | 46.8 | 45.5 | 46.2 | 46.2 |
| % Main 2 | 26.1 | 26.9 | 26.3 | 27.3 | 27.0 | 26.7 | 27.0 | 27.1 | 27.0 |
| % Basic | 14.6 | 12.5 | 13.0 | 12.6 | 12.4 | 12.8 | 13.3 | 12.7 | 12.8 |

TABLE 35
Sotatercept Surfactant Screen (Triton-X in Citrate)

| | 50 mg/mL Sotatercept in T-100, 10 mM Citrate, | | | | | | | | |
|---|---|---|---|---|---|---|---|---|---|
| | 8% (weight/volume) Sucrose, pH 5.8 | | | | | | | | |
| | 2 mg/mL T-100 | | | 0.2 mg/mL T-100 | | | 0.02 mg/mL T-100 | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | 54.25 | 56.55 | 52.35 | 55.61 | 54.53 | 52.93 | 56.25 | 53.24 | 55.06 |
| (mg/mL) | | | | | | | | | |
| pH | 5.61 | 5.72 | 5.73 | 5.19 | 6.07 | 4.33 | 5.87 | 6.00 | 5.06 |
| Turbidity | 0.180 | 0.190 | 0.195 | 0.205 | 0.220 | 0.200 | 0.120 | 0.180 | 0.140 |
| A₃₅₀₋₅₀₀ | | | | | | | | | |

UP-SEC

| | | | | | | | | | |
|---|---|---|---|---|---|---|---|---|---|
| % HMW | 1.4 | 1.4 | 1.4 | 1.4 | 1.4 | 1.4 | 1.4 | 1.4 | 1.4 |
| % Monomer | 93.2 | 93.3 | 93.1 | 93.3 | 93.3 | 93.2 | 93.1 | 93.2 | 93.1 |
| % LMW | 5.4 | 5.3 | 5.4 | 5.3 | 5.3 | 5.4 | 5.5 | 5.4 | 5.5 |

cIEF

| | | | | | | | | | |
|---|---|---|---|---|---|---|---|---|---|
| % Acidic | 14.0 | 13.8 | 13.8 | 14.3 | 13.7 | 13.9 | 13.8 | 13.9 | 13.9 |
| % Main 1 | 45.9 | 46.8 | 46.5 | 45.6 | 46.3 | 46.4 | 46.1 | 46.4 | 46.4 |
| % Main 2 | 27.0 | 27.0 | 27.0 | 27.3 | 27.3 | 26.9 | 27.1 | 27.2 | 27.0 |
| % Basic | 13.1 | 12.4 | 12.7 | 12.8 | 12.7 | 12.8 | 13.0 | 12.4 | 12.8 |

TABLE 36
Sotatercept Liquid Feasibility Stability (Controls in Citrate)

| | 50 mg/mL Sotatercept in 10 mM Citrate, | | | | | |
|---|---|---|---|---|---|---|
| | 8% (weight/volume) Sucrose, pH 5.8 | | | | | |
| | No Surfactant | | | 0.2 mg/mL PS-80 | | |
| | No Agit | Agitation at RT | | No Agit | Agitation at RT | |
| Assay | Control | 4 d | 7 d | Control | 4 d | 7 d |
| Conc. (mg/mL) | 53.46 | 55.66 | 53.75 | NT | 55.4 | 52.81 |
| pH | 5.79 | 5.61 | 4.56 | NT | 5.76 | 6.25 |
| Turbidity | 0.193 | 0.255 | 0.210 | NT | 0.185 | 0.115 |
| A₃₅₀₋₅₀₀ | | | | | | |

UP-SEC

| | | | | | | |
|---|---|---|---|---|---|---|
| % HMW | 1.4 | 1.4 | 1.4 | NT | 1.4 | 1.4 |
| % Monomer | 93.2 | 93.2 | 93.2 | NT | 93.2 | 93.3 |
| % LMW | 5.4 | 5.4 | 5.4 | NT | 5.4 | 5.3 |

cIEF

| | | | | | | |
|---|---|---|---|---|---|---|
| % Acidic | 14.0 | 14.5 | 14.2 | NT | 14.1 | 14.2 |
| % Main 1 | 45.8 | 45.7 | 46.3 | NT | 46.0 | 46.1 |
| % Main 2 | 27.4 | 27.0 | 26.6 | NT | 27.3 | 26.9 |
| % Basic | 12.9 | 12.9 | 13.0 | NT | 12.6 | 12.8 |

The results show that sotatercept is stable against agitation stress in citrate buffer and there is no significant difference between the surfactants tested when comparing charge species, monomer species, aggregates, or fragmentation.

Tables 37-41 show surfactant stability studies for sotatercept carried out by agitation stress by shaking the vials containing sotatercept in 10 mM histidine buffer, 8% sucrose with varying levels of surfactants. The surfactants screened were polaxamer188, sodium dodecyl sulfate (SDS), N-dodecyl-β-D-maltoside (DDM), polysorbate 20 and triton X at 0.02 mg/mL, 0.2 mg/mL and 2 mg/mL for 4 days and 7 days. Table 42 show surfactant stability studies for the control containing sotatercept in 10 mM histidine buffer, 800 sucrose and no surfactant.

TABLE 37
Sotatercept Surfactant Screen (N-dodecyl-β-D-maltoside in Histidine)

| | 50 mg/mL Sotatercept in DDM, 10 mM Histidine, | | | | | | | | |
|---|---|---|---|---|---|---|---|---|---|
| | 8% (weight/volume) Sucrose, pH 5.8 | | | | | | | | |
| | 2 mg/mL DDM | | | 0.2 mg/mL DDM | | | 0.02 mg/mL DDM | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | NT | NT | NT | 50.96 | 50.18 | 51.73 | 51.56 | 50.14 | 52.64 |
| (mg/mL) | | | | | | | | | |
| pH | 5.84 | 5.67 | 5.89 | 5.86 | 5.61 | 5.98 | 5.81 | 5.61 | 6.00 |
| Turbidity | NT | NT | NT | 0.165 | 0.165 | 0.285 | 0.180 | 0.130 | 0.290 |
| A₃₅₀₋₅₀₀ | | | | | | | | | |

UP-SEC

| | | | | | | | | | |
|---|---|---|---|---|---|---|---|---|---|
| % HMW | 1.1 | 1.2 | 1.1 | 1.0 | 1.0 | 1.1 | 1.0 | 1.1 | 1.6 |
| % Monomer | 93.3 | 93.7 | 93.5 | 91.2 | 92.6 | 92.0 | 92.3 | 93.7 | 91.4 |
| % LMW | 5.6 | 5.0 | 5.4 | 7.7 | 6.4 | 6.9 | 6.7 | 5.2 | 7.0 |

cIEF

| | | | | | | | | | |
|---|---|---|---|---|---|---|---|---|---|
| % Acidic | 15.4 | 14.7 | 14.9 | 14.9 | 14.4 | 15.1 | 15.2 | 15.0 | 15.4 |
| % Main 1 | 46.3 | 47.4 | 48.2 | 46.2 | 47.2 | 48.0 | 46.9 | 48.9 | 48.0 |
| % Main 2 | 25.5 | 25.3 | 25.8 | 25.5 | 25.8 | 25.3 | 25.3 | 25.3 | 25.2 |
| % Basic | 12.9 | 12.6 | 11.1 | 13.4 | 12.6 | 11.6 | 12.6 | 10.7 | 11.4 |

TABLE 38
Sotatercept Surfactant Screen (Polaxamer-188 in Histidine)

| | 50 mg/mL Sotatercept in P-188, 10 mM Histidine, | | | | | | | | |
|---|---|---|---|---|---|---|---|---|---|
| | 8% (weight/volume) Sucrose, pH 5.8 | | | | | | | | |
| | 2 mg/mL P-188 | | | 0.2 mg/mL P-188 | | | 0.02 mg/mL P-188 | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. | 51.40 | 49.36 | 51.51 | 52.46 | 50.20 | 51.23 | 51.22 | 50.28 | 50.50 |
| (mg/mL) | | | | | | | | | |
| pH | 5.82 | 5.75 | 5.90 | 5.93 | 5.71 | 6.03 | 5.85 | 5.75 | 5.92 |
| Turbidity | 0.170 | 0.160 | 0.185 | 0.220 | 0.160 | 0.250 | 0.105 | 0.220 | 0.290 |
| A₃₅₀₋₅₀₀ | | | | | | | | | |

UP-SEC

| | | | | | | | | | |
|---|---|---|---|---|---|---|---|---|---|
| % HMW | 1.1 | 1.1 | 1.0 | 1.1 | 1.1 | 1.1 | 1.0 | 1.1 | 1.0 |
| % Monomer | 92.9 | 93.3 | 92.2 | 93.2 | 93.4 | 93.2 | 92.1 | 93.3 | 92.2 |
| % LMW | 6.0 | 5.6 | 6.8 | 5.7 | 5.5 | 5.8 | 6.9 | 5.7 | 6.7 |

cIEF

| | | | | | | | | | |
|---|---|---|---|---|---|---|---|---|---|
| % Acidic | 15.2 | 14.9 | 14.7 | 15.2 | 14.6 | 15.0 | 15.1 | 14.7 | 14.2 |
| % Main 1 | 46.1 | 47.0 | 46.8 | 46.9 | 49.3 | 47.0 | 46.5 | 47.6 | 47.6 |
| % Main 2 | 25.9 | 25.3 | 25.7 | 25.3 | 25.4 | 25.6 | 25.5 | 25.1 | 25.8 |
| % Basic | 12.8 | 12.8 | 12.8 | 12.6 | 10.7 | 12.4 | 12.9 | 12.5 | 12.4 |

TABLE 39
Sotatercept Surfactant Screen (Polysorbate-20 in Histidine)

| | 50 mg/mL Sotatercept in PS-20, 10 mM Histidine, 8% (weight/volume) Sucrose, pH 5.8 | | | | | | | | |
|---|---|---|---|---|---|---|---|---|---|
| | 2 mg/mL PS-20 | | | 0.2 mg/mL PS-20 | | | 0.02 mg/mL PS-20 | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. (mg/mL) | 50.19 | 50.30 | 50.65 | 48.56 | 50.13 | 52.21 | 51.48 | 51.34 | 51.38 |
| pH | 5.82 | 5.70 | 5.94 | 5.82 | 5.73 | 6.02 | 5.85 | 5.72 | 5.90 |
| Turbidity A₃₅₀₋₅₀₀ | 0.115 | 0.125 | 0.140 | 0.145 | 0.165 | 0.215 | 0.195 | 0.215 | 0.150 |

UP-SEC

| | | | | | | | | | |
|---|---|---|---|---|---|---|---|---|---|
| % HMW | 1.0 | 1.1 | 1.1 | 1.0 | 1.1 | 1.0 | 1.0 | 1.1 | 1.0 |
| % Monomer | 92.2 | 93.1 | 91.6 | 92.2 | 92.9 | 92.0 | 91.9 | 92.6 | 91.8 |
| % LMW | 6.8 | 5.8 | 7.3 | 6.8 | 6.0 | 6.9 | 7.1 | 6.3 | 7.2 |

cIEF

| | | | | | | | | | |
|---|---|---|---|---|---|---|---|---|---|
| % Acidic | 15.4 | 14.8 | 14.9 | 15.4 | 14.9 | 15.1 | 15.2 | 15.0 | 15.0 |
| % Main 1 | 46.0 | 47.2 | 47.2 | 45.9 | 48.0 | 47.2 | 46.7 | 47.5 | 47.3 |
| % Main 2 | 25.4 | 25.3 | 25.5 | 25.0 | 25.5 | 25.5 | 25.7 | 25.4 | 25.3 |
| % Basic | 13.3 | 12.6 | 12.4 | 13.6 | 11.6 | 12.2 | 12.3 | 12.1 | 12.4 |

TABLE 40
Sotatercept Surfactant Screen (Sodium Dodecyl Sulfate in Histidine)

| | 50 mg/mL Sotatercept in SDS, 10 mM Histidine, 8% (weight/volume) Sucrose, pH 5.8 | | | | | | | | |
|---|---|---|---|---|---|---|---|---|---|
| | 2 mg/mL SDS | | | 0.2 mg/mL SDS | | | 0.02 mg/mL SDS | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. (mg/mL) | 52.28 | 49.70 | 51.31 | 51.65 | 50.35 | 52.18 | 51.16 | 50.56 | 51.78 |
| pH | 6.05 | 5.76 | 6.21 | 5.86 | 5.59 | 5.99 | 5.84 | 5.62 | 5.94 |
| Turbidity A₃₅₀₋₅₀₀ | 0.175 | 0.195 | 0.330 | 0.155 | 0.155 | 0.285 | 0.155 | 0.140 | 0.265 |

UP-SEC

| | | | | | | | | | |
|---|---|---|---|---|---|---|---|---|---|
| % HMW | 4.6 | 4.4 | 4.6 | 1.4 | 1.2 | 1.2 | 1.1 | 1.0 | 1.1 |
| % Monomer | 88.3 | 88.6 | 88.1 | 93.0 | 92.0 | 91.7 | 93.2 | 93.5 | 93.0 |
| % LMW | 7.1 | 7.0 | 7.4 | 5.6 | 6.8 | 7.0 | 5.7 | 5.5 | 5.9 |

cIEF

| | | | | | | | | | |
|---|---|---|---|---|---|---|---|---|---|
| % Acidic | 15.1 | 15.3 | 15.2 | 14.9 | 14.9 | 15.4 | 14.8 | 15.5 | 14.9 |
| % Main 1 | 45.2 | 46.6 | 46.5 | 46.7 | 47.3 | 47.1 | 47.0 | 47.2 | 47.5 |
| % Main 2 | 25.1 | 24.9 | 24.8 | 25.6 | 25.5 | 25.5 | 25.5 | 25.1 | 26.0 |
| % Basic | 14.6 | 13.2 | 13.4 | 12.7 | 12.3 | 12.0 | 12.8 | 12.3 | 11.6 |

TABLE 41
Sotatercept Surfactant Screen (Triton-X in Histidine)

| | 50 mg/mL Sotatercept in T-100, 10 mM Histidine, 8% (weight/volume) Sucrose, pH 5.8 | | | | | | | | |
|---|---|---|---|---|---|---|---|---|---|
| | 2 mg/mL T-100 | | | 0.2 mg/mL T-100 | | | 0.02 mg/mL T-100 | | |
| | No Agit | Agitation at RT | | No Agit | Agitation at RT | | No Agit | Agitation at RT | |
| Assay | Control | 4 d | 7 d | Control | 4 d | 7 d | Control | 4 d | 7 d |
| Conc. (mg/mL) | 50.73 | 49.98 | 51.27 | 51.95 | 50.28 | 51.69 | 50.72 | 50.19 | 51.44 |
| pH | 5.86 | 5.63 | 5.95 | 5.85 | 5.62 | 5.97 | 5.94 | 5.63 | 6.02 |
| Turbidity A₃₅₀₋₅₀₀ | 0.130 | 0.195 | 0.355 | 0.150 | 0.165 | 0.205 | 0.130 | 0.160 | 0.255 |

UP-SEC

| | | | | | | | | | |
|---|---|---|---|---|---|---|---|---|---|
| % HMW | 1.0 | 1.0 | 1.0 | 1.0 | 1.0 | 1.0 | 1.1 | 1.0 | 1.1 |
| % Monomer | 91.7 | 92.0 | 91.6 | 92.3 | 92.8 | 91.8 | 93.1 | 92.6 | 92.1 |
| % LMW | 7.4 | 7.0 | 7.3 | 6.7 | 6.2 | 7.2 | 5.8 | 6.3 | 6.8 |

cIEF

| | | | | | | | | | |
|---|---|---|---|---|---|---|---|---|---|
| % Acidic | 15.8 | 14.5 | 14.9 | 15.2 | 15.1 | 15.2 | 15.1 | 14.9 | 14.9 |
| % Main 1 | 45.8 | 49.0 | 46.1 | 46.3 | 47.2 | 46.3 | 46.7 | 47.5 | 47.2 |
| % Main 2 | 25.5 | 25.6 | 25.3 | 25.6 | 25.0 | 25.5 | 25.9 | 25.3 | 25.8 |
| % Basic | 12.9 | 10.9 | 13.7 | 12.9 | 12.7 | 13.0 | 12.3 | 12.3 | 12.1 |

TABLE 42
Sotatercept Surfactant Screen (Controls in Histidine)

| | 50 mg/mL Sotatercept in 10 mM Histidine, 8% (weight/volume) Sucrose, pH 5.8 | | | | | |
|---|---|---|---|---|---|---|
| | No Surfactant Control | | | 0.2 mg/mL PS-80 | | |
| | No Agit | Agitation at RT | | No Agit | Agitation at RT | |
| Assay | Control | 4 d | 7 d | Control | 4 d | 7 d |
| Conc. (mg/mL) | 51.42 | 50.67 | 51.45 | 50.87 | 50.48 | 51.45 |
| pH | 5.83 | 5.67 | 5.98 | 5.83 | 5.62 | 5.91 |
| Turbidity A₃₅₀₋₅₀₀ | 0.130 | 0.185 | 0.205 | 0.160 | 0.170 | 0.205 |

UP-SEC

| | | | | | | |
|---|---|---|---|---|---|---|
| % HMW | 1.0 | 1.1 | 1.0 | 1.0 | 1.1 | 1.0 |
| % Monomer | 92.1 | 92.0 | 91.8 | 91.5 | 93.3 | 91.9 |
| % LMW | 6.9 | 6.9 | 7.2 | 7.5 | 5.7 | 7.0 |

cIEF

| | | | | | | |
|---|---|---|---|---|---|---|
| % Acidic | 14.9 | 14.9 | 14.9 | 15.4 | 14.8 | 15.2 |
| % Main 1 | 47.2 | 48.4 | 47.2 | 45.4 | 47.0 | 47.0 |
| % Main 2 | 25.4 | 25.5 | 25.6 | 26.0 | 25.6 | 25.8 |
| % Basic | 12.6 | 11.2 | 12.2 | 13.1 | 12.5 | 11.9 |

The results show that sotatercept is stable against agitation stress when formulated with the surfactants tested in histidine buffer. At high SDS concentrations, slightly higher high molecular weight species were detected. There is no significant difference between the surfactants tested when looking at charge species after agitation stress.

### Chelator Studies

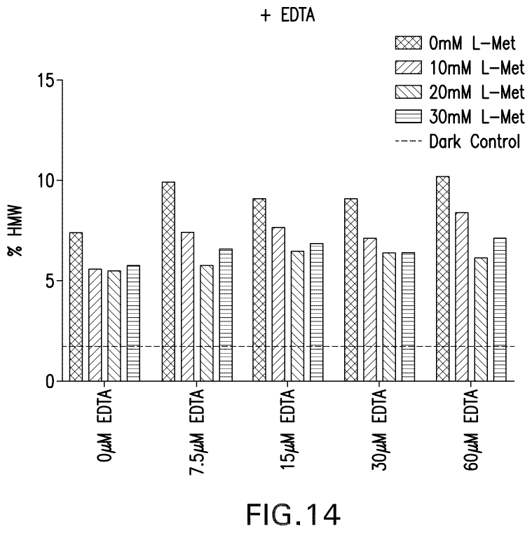

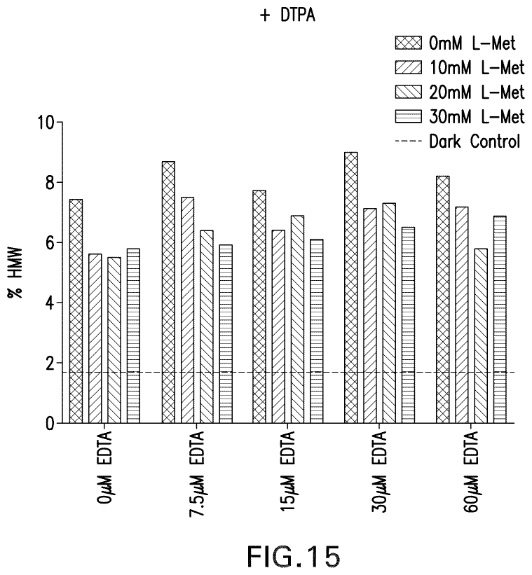

Formulations comprising sotatercept (SEQ ID NO:32) in 10 mM citrate buffer, 8% sucrose and 0.02% polysorbate 80 at a pH of 5.8 with different levels (0 mM, 10 mM, 20 mM, 30 mM) of L-methionine were exposed to light stress in a photostability chamber in presence of different levels of either DTPA or EDTA as chelating agents (0 μM, 7.5 μM, 15 μM, 30 μM, 60 μM). The stressed samples along with dark controls were analyzed for high molecular weight formation (using UPSEC) and showed that addition of L-methionine reduced % HMW formation and there was no negative effect of adding chelators. Results are shown in FIGS. 14 and 15. Hence either of the chelators can be used in the formulation.

Additionally, sotatercept was formulated with 10 mM citrate buffer or 10 mM histidine buffer, 8% sucrose and 0.02% polysorbate 80 at a pH of 5.8 with different levels (0 mM, 5 mM, 10 mM, 50 mM) of dimercaprol. The samples were exposed to light stress in a photostability chamber. The stressed samples along with dark controls were analyzed for high molecular weight formation (using UPSEC) and showed that addition of dimercaprol. The results of several tests of formulations comprising sotatercept, 10 mM citrate buffer, 8% sucrose and 0.02% polysorbate 80 at a pH of 5.8 with different levels (0 mM, 5 mM, 10 mM, 50 mM) of dimercaprol are shown in Table 43 with the dark control results shown in Table 44. The results of several tests of formulations comprising sotatercept, 10 mM histidine buffer, 8% sucrose and 0.02% polysorbate 80 at a pH of 5.8 with different levels (0 mM, 5 mM, 10 mM, 50 mM) of dimercaprol are shown in Table 45 with the dark control results shown in Table 46.

TABLE 43
Sotatercept Antioxidant Screen (Dimercaprol in Citrate)

| | 50 mg/mL Sotatercept in10 mM Citrate, 80 mg/ml Sucrose, 0.2 mg/mL Polysorbate 80, pH 5.8 | | | | | | | | |
|---|---|---|---|---|---|---|---|---|---|
| | 50 mg/mL Dimercaprol | | | 10 mg/mL Dimercaprol | | | 5 mg/mL Dimercaprol | | |
| | Visible Light | | | Visible Light | | | Visible Light | | |
| Assay | 0.2X | 0.5X | DC | 0.2X | 0.5X | DC | 0.2X | 0.5X | DC |
| Conc. (mg/mL) | NT | NT | NT | 50.69 | 53.61 | 53.84 | 53.57 | 54.49 | 54.88 |
| pH | 6.09 | 6.13 | 6.18 | 5.81 | 5.89 | 5.99 | 5.79 | 5.89 | 5.82 |
| Turbidity A₃₅₀₋₅₀₀ | NT | NT | NT | 0.290 | 0.240 | .0245 | 0.235 | .0335 | 0.180 |

UP-SEC

| | | | | | | | | | |
|---|---|---|---|---|---|---|---|---|---|
| % HMW | 67.7 | 66.4 | 66.7 | 50.3 | 71.3 | 58.9 | 7.8 | 15.3 | 1.8 |
| % Monomer | 22.8 | 19.7 | 23.7 | 40.8 | 22.6 | 32.9 | 85.8 | 78.4 | 91.3 |
| % LMW | 9.5 | 13.9 | 9.6 | 8.9 | 6.1 | 8.2 | 6.5 | 6.3 | 6.9 |

cIEF

| | | | | | | | | | |
|---|---|---|---|---|---|---|---|---|---|
| % Acidic | NT | NT | NT | 6.1 | 4.2 | 5.2 | 15.9 | 17.0 | 14.0 |
| % Main 1 | NT | NT | NT | 17.4 | 8.3 | 14.2 | 38.8 | 33.7 | 44.3 |
| % Main 2 | NT | NT | NT | 10.0 | 4.4 | 8.8 | 28.3 | 28.6 | 27.3 |
| % Basic | NT | NT | NT | 66.6 | 83.0 | 71.8 | 17.0 | 20.7 | 14.4 |

TABLE 44
Sotatercept Antioxidant Screen (Controls in Citrate)

| | 50 mg/mL Sotatercept in10 mM Citrate, 80 mg/ml Sucrose, 0.2 mg/mL Polysorbate 80, pH 5.8 | | | | | |
|---|---|---|---|---|---|---|
| | No Antioxidant Control | | | 20 mM L-Methionine | | |
| | Visible Light | | | Visible Light | | |
| Assay | 0.2X | 0.5X | DC | 0.2X | 0.5X | DC |
| Conc. (mg/mL) | 52.99 | 43.52 | 53.09 | 52.94 | 55.71 | 53.42 |
| pH | 5.82 | 5.92 | 5.77 | 6.03 | 5.92 | 5.85 |
| Turbidity A₃₅₀₋₅₀₀ | 0.160 | 0.310 | 0.170 | 0.225 | 0.235 | 0.220 |

UP-SEC

| | | | | | | |
|---|---|---|---|---|---|---|
| % HMW | 8.9 | 17.7 | 1.4 | 7.8 | 16.1 | 1.4 |
| % Monomer | 86.0 | 77.4 | 93.1 | 87.0 | 78.8 | 93.1 |
| % LMW | 5.1 | 5.0 | 5.4 | 5.2 | 5.1 | 5.5 |

cIEF

| | | | | | | |
|---|---|---|---|---|---|---|
| % Acidic | 15.4 | 23.2 | 13.8 | 15.0 | 15.5 | 13.9 |
| % Main 1 | 39.4 | 33.9 | 45.3 | 40.7 | 35.5 | 44.8 |
| % Main 2 | 28.8 | 22.3 | 27.3 | 27.7 | 27.4 | 27.7 |
| % Basic | 16.4 | 20.5 | 13.5 | 16.6 | 21.6 | 13.7 |

TABLE 45
Sotatercept Antioxidant Screen (Dimercaprol in Histidine)

| | 50 mg/mL Sotatercept in10 mM Histidine, 80 mg/ml Sucrose, 0.2 mg/mL Polysorbate 80, pH 5.8 | | | | | | | | |
|---|---|---|---|---|---|---|---|---|---|
| | 50 mg/mL Dimercaprol | | | 10 mg/mL Dimercaprol | | | 5 mg/mL Dimercaprol | | |
| | Visible Light | | | Visible Light | | | Visible Light | | |
| Assay | 0.2X | 0.5X | DC | 0.2X | 0.5X | DC | 0.2X | 0.5X | DC |
| Conc. (mg/mL) | NT | NT | NT | 53.48 | 52.25 | 51.49 | 51.51 | 51.19 | 50.65 |
| pH | NT | NT | NT | 5.82 | 5.90 | 5.85 | 5.81 | 5.79 | 5.80 |
| Turbidity A₃₅₀₋₅₀₀ | NT | NT | NT | 1.220 | 0.490 | 0.780 | 0.290 | 0.350 | 0.175 |

UP-SEC

| | | | | | | | | | |
|---|---|---|---|---|---|---|---|---|---|
| % HMW | NT | NT | NT | 53.3 | 71.2 | 58.9 | 7.2 | 16.3 | 1.1 |
| % Monomer | NT | NT | NT | 38.1 | 24.6 | 32.9 | 86.6 | 77.8 | 92.8 |
| % LMW | NT | NT | NT | 8.6 | 4.2 | 8.2 | 6.2 | 5.8 | 6.1 |

cIEF

| | | | | | | | | | |
|---|---|---|---|---|---|---|---|---|---|
| % Acidic | NT | NT | NT | NT | NT | NT | 17.2 | 17.1 | 15.1 |
| % Main 1 | NT | NT | NT | NT | NT | NT | 43.2 | 41.1 | 46.0 |
| % Main 2 | NT | NT | NT | NT | NT | NT | 26.1 | 24.6 | 25.8 |
| % Basic | NT | NT | NT | NT | NT | NT | 13.5 | 17.2 | 13.2 |

TABLE 46
Sotatercept Antioxidant Screen (Controls in Histidine)

| | 50 mg/mL Sotatercept in10 mM Citrate, 80 mg/ml Sucrose, 0.2 mg/mL Polysorbate 80, pH 5.8 | | | | | |
|---|---|---|---|---|---|---|
| | No Antioxidant Control | | | 20 mM L-Methionine | | |
| | Visible Light | | | Visible Light | | |
| Assay | 0.2X | 0.5X | DC | 0.2X | 0.5X | DC |
| Conc. (mg/mL) | 51.24 | 51.82 | 50.25 | 50.43 | 51.67 | 50.93 |
| pH | 5.79 | 5.78 | 5.85 | 5.80 | 5.80 | 5.81 |
| Turbidity A₃₅₀₋₅₀₀ | 0.295 | 0.355 | 0.180 | 0.255 | 0.275 | 0.170 |

UP-SEC

| | | | | | | |
|---|---|---|---|---|---|---|
| % HMW | 7.2 | 15.9 | 1.1 | 6.8 | 15.0 | 1.1 |
| % Monomer | 86.8 | 77.9 | 93.0 | 87.5 | 78.9 | 93.3 |
| % LMW | 6.0 | 6.3 | 5.9 | 5.7 | 6.1 | 5.7 |

cIEF

| | | | | | | |
|---|---|---|---|---|---|---|
| % Acidic | 17.4 | 16.9 | 15.0 | 16.5 | 16.5 | 15.5 |
| % Main 1 | 42.9 | 41.0 | 46.4 | 42.1 | 41.3 | 45.9 |
| % Main 2 | 26.1 | 24.9 | 25.5 | 25.8 | 24.7 | 25.2 |
| % Basic | 13.7 | 17.2 | 13.2 | 15.7 | 17.6 | 13.4 |

The results show that inclusion of low concentrations of dimercaprol as an antioxidant was able to provide comparable protection to control samples under light stress in both citrate and histidine buffer. Inclusion of higher amounts of dimercaprol with light stress resulted in increased charge species and greater loss of monomer content indicating the formulation was not compatible with higher dimercaprol content as an antioxidant.

### Stabilizer Screen

The behavior of sotatercept (SEQ ID NO: 32) was analyzed in various stabilizers. The stabilizers tested were carboxy methyl cellulose (CMC), dextrose, polyethylene glycol (PEG), albumin, kelptose, proline, trehalose, mannitol, and dextran. Tables 47-56 show the results of sotatercept formulations comprising 10 mM citrate, 20 mM L-Methionine, 0.2 mg/mL and Polysorbate 80 at pH 5.8 with the various surfactants. Table 57 shows the results of the citrate control formulation without surfactants. Tables 58-67 show the results of sotatercept formulations comprising 10 mM histidine, 20 mM L-Methionine, 0.2 mg/mL and Polysorbate 80 at pH 5.8 with the various surfactants. Table 68 shows the results of a control formulation without surfactants. The formulations were thermally stressed at 50° C. for up to two weeks and tested for biophysical stability (turbidity), subvisible particle formation (measured by Aura), high molecular weight formation (via UPSEC), aggregation (via UPSEC), and fragmentation (via UPSEC).

TABLE 47
Sotatercept Stabilizer Screen (Polyethylene Glycol - 300 in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 50 mg/mL PEG-300 | | | 25 mg/mL PEG-300 | | | 5 mg/mL PEG-300 | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.07 | 58.01 | 51.8 | 54.24 | 56.61 | 51.65 | 54.43 | 56.40 | 52.02 |
| pH | 5.47 | 6.01 | 5.90 | 5.95 | 5.96 | 5.89 | 6.00 | 6.03 | 5.94 |
| Turbidity A₃₅₀₋₅₀₀ | 0.205 | 0.230 | 0.225 | 0.260 | 0.215 | 0.195 | 0.255 | 0.260 | 0.215 |

UP-SEC

| | | | | | | | | | |
|---|---|---|---|---|---|---|---|---|---|
| % HMW | 1.7 | 4.2 | 5.2 | 1.6 | 3.7 | 4.6 | 1.5 | 3.3 | 4.2 |
| % Monomer | 93.2 | 89.6 | 87.5 | 93.3 | 90.0 | 88.0 | 93.4 | 90.4 | 88.5 |
| % LMW | 5.1 | 6.2 | 7.3 | 5.1 | 6.3 | 7.4 | 5.1 | 6.3 | 7.3 |

cIEF

| | | | | | | | | | |
|---|---|---|---|---|---|---|---|---|---|
| % Acidic | 13.8 | 15.6 | 16.3 | 13.6 | 15.1 | 16.3 | 13.3 | 14.1 | 15.0 |
| % Main 1 | 46.8 | 34.9 | 28.5 | 47.5 | 35.5 | 29.5 | 47.6 | 38.8 | 31.5 |
| % Main 2 | 26.5 | 25.7 | 23.6 | 26.6 | 25.1 | 23.4 | 26.8 | 24.3 | 23.6 |
| % Basic | 13.0 | 23.8 | 31.7 | 12.3 | 24.3 | 30.8 | 12.3 | 22.7 | 29.9 |

TABLE 48
Sotatercept Stabilizer Screen (Polyethylene Glycol-3350 in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 50 mg/mL PEG-3350 | | | 25 mg/mL PEG-3350 | | | 5 mg/mL PEG-3350 | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 56.31 | 60.66 | 42.31 | 55.70 | 57.03 | 39.46 | 54.33 | 56.29 | 53.28 |
| pH | 5.99 | NT | 6.01 | 6.12 | 5.92 | 6.03 | 5.72 | 6.00 | 6.00 |
| Turbidity A₃₅₀₋₅₀₀ | 0.290 | 0.290 | 0.105 | 0.290 | 0.270 | 0.080 | 0.245 | 0.250 | .0235 |

UP-SEC

| | | | | | | | | | |
|---|---|---|---|---|---|---|---|---|---|
| % HMW | 1.7 | 4.4 | 5.5 | 1.6 | 3.9 | 4.9 | 1.5 | 3.3 | 4.3 |
| % Monomer | 93.1 | 89.6 | 87.5 | 93.3 | 90.0 | 87.9 | 93.4 | 90.4 | 88.5 |
| % LMW | 5.2 | 6.0 | 7.0 | 5.1 | 6.1 | 7.2 | 5.1 | 6.2 | 7.2 |

cIEF

| | | | | | | | | | |
|---|---|---|---|---|---|---|---|---|---|
| % Acidic | 13.7 | 14.2 | 15.1 | 13.8 | 14.3 | 15.1 | 13.8 | 14.1 | 15.1 |
| % Main 1 | 47.4 | 37.2 | 30.5 | 47.5 | 37.5 | 31.8 | 47.3 | 38.7 | 31.5 |
| % Main 2 | 26.6 | 24.9 | 23.6 | 26.5 | 24.9 | 23.6 | 26.5 | 24.3 | 23.2 |
| % Basic | 12.4 | 23.7 | 30.8 | 12.2 | 23.3 | 29.6 | 12.4 | 22.9 | 30.2 |

TABLE 49
Sotatercept Stabilizer Screen (Carboxy Methyl Cellulose (CMC) in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 10 mg/mL CMC | | | 5 mg/mL CMC | | | 1 mg/mL CMC | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.90 | 55.39 | 52.88 | 54.99 | 60.16 | 52.42 | 54.54 | 54.39 | 52.37 |
| pH | 5.83 | 5.98 | 5.92 | 5.87 | 5.97 | 5.90 | 5.73 | 5.83 | 5.90 |
| Turbidity A₃₅₀₋₅₀₀ | 0.245 | 0.190 | 0.215 | 0.275 | 0.225 | 0.240 | 0.255 | 0.225 | 0.140 |

UP-SEC

| | | | | | | | | | |
|---|---|---|---|---|---|---|---|---|---|
| % HMW | 1.5 | 5.3 | 6.4 | 1.5 | 5.1 | 6.4 | 1.5 | 4.2 | 5.3 |
| % Monomer | 93.3 | 88.6 | 86.6 | 93.3 | 88.7 | 86.4 | 93.4 | 89.7 | 87.7 |
| % LMW | 5.1 | 6.1 | 6.9 | 5.2 | 6.2 | 7.2 | 5.1 | 6.2 | 7.1 |

cIEF

| | | | | | | | | | |
|---|---|---|---|---|---|---|---|---|---|
| % Acidic | 10.7 | 11.9 | 14.2 | 12.2 | 12.9 | 14.7 | 13.6 | 14.0 | 15.3 |
| % Main 1 | 51.5 | 41.5 | 33.3 | 48.8 | 40.0 | 32.7 | 47.5 | 39.8 | 32.3 |
| % Main 2 | 25.8 | 24.3 | 24.1 | 26.9 | 25.2 | 24.1 | 26.7 | 24.7 | 24.1 |
| % Basic | 12.0 | 22.2 | 28.3 | 121 | 21.9 | 28.5 | 12.3 | 21.5 | 28.3 |

TABLE 50
Sotatercept Stabilizer Screen (Dextran in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 20 mg/mL Dextran | | | 10 mg/mL Dextran | | | 5 mg/mL Dextran | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.81 | 57.65 | 52.91 | 55.89 | 57.38 | 52.78 | 55.10 | 56.22 | 53.59 |
| pH | 5.87 | 5.8 | 5.89 | 5.89 | 5.82 | 6.05 | 5.83 | 5.89 | 5.92 |
| Turbidity A₃₅₀₋₅₀₀ | 0.290 | 0.285 | 0.140 | 0.250 | 0.280 | 0.290 | 0.225 | 0.145 | 0.220 |

UP-SEC

| | | | | | | | | | |
|---|---|---|---|---|---|---|---|---|---|
| % HMW | 1.8 | 3.9 | 5.0 | 1.6 | 3.7 | 4.6 | 1.5 | 3.4 | 4.4 |
| % Monomer | 93.1 | 89.9 | 87.9 | 93.0 | 90.1 | 88.2 | 93.3 | 90.4 | 88.4 |
| % LMW | 5.1 | 6.1 | 7.2 | 5.4 | 6.2 | 7.2 | 5.2 | 6.2 | 7.2 |

cIEF

| | | | | | | | | | |
|---|---|---|---|---|---|---|---|---|---|
| % Acidic | 13.7 | 14.0 | 15.0 | 14.3 | 13.9 | 15.3 | 13.7 | 14.3 | 15.2 |
| % Main 1 | 47.7 | 37.8 | 31.7 | 46.5 | 37.9 | 31.4 | 47.3 | 37.3 | 31.6 |
| % Main 2 | 26.3 | 25.0 | 23.5 | 25.8 | 25.0 | 23.3 | 26.6 | 24.8 | 23.2 |
| % Basic | 12.4 | 23.2 | 29.9 | 13.4 | 23.2 | 30.0 | 12.4 | 23.7 | 30.0 |

TABLE 51
Sotatercept Stabilizer Screen (Dextrose in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 80 mg/mL Dextrose | | | 40 mg/mL Dextrose | | | 5 mg/mL Dextrose | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.02 | 57.2 | 54.26 | 54.60 | 56.09 | 54.21 | 54.14 | 57.48 | 52.77 |
| pH | 5.82 | 5.97 | 5.97 | 5.94 | 6.09 | 5.95 | 5.76 | 5.85 | 6.01 |
| Turbidity A₃₅₀₋₅₀₀ | 0.295 | 0.345 | 0.780 | 0.250 | 0.250 | 0.520 | 0.230 | 0.205 | 0.255 |

UP-SEC

| | | | | | | | | | |
|---|---|---|---|---|---|---|---|---|---|
| % HMW | 1.5 | 3.6 | 7.7 | 1.5 | 3.4 | 6.1 | 1.5 | 3.3 | 4.4 |
| % Monomer | 93.4 | 90.1 | 85.3 | 93.4 | 90.3 | 86.4 | 93.5 | 90.5 | 88.4 |
| % LMW | 5.1 | 6.3 | 7.0 | 5.2 | 6.3 | 7.5 | 5.1 | 6.3 | 7.2 |

cIEF

| | | | | | | | | | |
|---|---|---|---|---|---|---|---|---|---|
| % Acidic | 13.9 | 18.7 | 27.4 | 13.8 | 16.6 | 22.3 | 13.7 | 14.7 | 15.7 |
| % Main 1 | 47.0 | 33.6 | 23.7 | 47.2 | 36.7 | 28.1 | 47.5 | 38.0 | 31.9 |
| % Main 2 | 26.9 | 30.4 | 31.2 | 26.8 | 28.3 | 28.1 | 26.6 | 25.0 | 24.1 |
| % Basic | 12.3 | 17.3 | 17.7 | 12.2 | 18.4 | 21.5 | 12.2 | 22.4 | 28.3 |

TABLE 52
Sotatercept Stabilizer Screen (Mannitol in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 80 mg/mL Mannitol | | | 40 mg/mL Mannitol | | | 5 mg/mL Mannitol | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.31 | 57.74 | 53.02 | 55.27 | 58.38 | 53.17 | 55.09 | 56.08 | 52.17 |
| pH | 5.77 | 6.00 | 5.86 | 5.74 | 5.93 | 5.94 | 6.01 | 6.02 | 5.91 |
| Turbidity A₃₅₀₋₅₀₀ | 0.260 | 0.200 | 0.210 | 0.145 | 0.160 | 0.215 | 0.270 | 0.155 | 0.175 |

UP-SEC

| | | | | | | | | | |
|---|---|---|---|---|---|---|---|---|---|
| % HMW | 1.5 | 2.9 | 3.5 | 1.5 | 3.0 | 3.8 | 1.5 | 3.2 | 4.1 |
| % Monomer | 93.5 | 90.8 | 89.1 | 93.5 | 90.7 | 89.0 | 93.5 | 90.6 | 88.7 |
| % LMW | 5.1 | 6.3 | 7.4 | 5.1 | 6.2 | 7.2 | 5.0 | 6.2 | 7.2 |

cIEF

| | | | | | | | | | |
|---|---|---|---|---|---|---|---|---|---|
| % Acidic | 13.7 | 14.0 | 14.5 | 13.5 | 14.1 | 15.2 | 13.9 | 14.4 | 14.9 |
| % Main 1 | 47.3 | 37.5 | 32.0 | 47.3 | 39.0 | 31.2 | 47.0 | 37.7 | 31.8 |
| % Main 2 | 26.7 | 25.0 | 23.4 | 26.7 | 24.3 | 23.6 | 26.9 | 25.0 | 23.5 |
| % Basic | 12.3 | 23.6 | 30.1 | 12.5 | 22.5 | 30.1 | 12.2 | 22.9 | 29.8 |

TABLE 53
Sotatercept Stabilizer Screen (Trehalose in Citrate)

| | 50 mg/mL Sotatercept in10 mM Citrate, 20 mM L-Methionine, 0.2 mg/mL Polysorbate 80, pH 5.8 | | | | | | | | |
|---|---|---|---|---|---|---|---|---|---|
| | 80 mg/mL Trehalose | | | 40 mg/mL Trehalose | | | 5 mg/mL Trehalose | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.05 | 56.64 | 52.76 | 52.91 | 56.43 | 54.54 | 55.01 | 56.04 | 53.10 |
| pH | 6.00 | 5.90 | 5.99 | 5.94 | 5.90 | 6.02 | 5.88 | 5.96 | 5.90 |
| Turbidity A₃₅₀₋₅₀₀ | 0.255 | 0.205 | 0.185 | 0.285 | 0.250 | 0.170 | 0.285 | 0.230 | 0.220 |

UP-SEC

| | | | | | | | | | |
|---|---|---|---|---|---|---|---|---|---|
| % HMW | 1.5 | 2.7 | 3.4 | 1.5 | 2.9 | 3.7 | 1.5 | 3.2 | 4.1 |
| % Monomer | 93.4 | 90.9 | 89.3 | 93.4 | 90.8 | 88.9 | 93.4 | 90.4 | 88.8 |
| % LMW | 5.2 | 6.4 | 7.4 | 5.2 | 6.3 | 7.4 | 5.1 | 6.3 | 7.1 |

cIEF

| | | | | | | | | | |
|---|---|---|---|---|---|---|---|---|---|
| % Acidic | 14.1 | 14.2 | 14.5 | 13.8 | 13.8 | 14.5 | 13.9 | 14.2 | 15.1 |
| % Main 1 | 47.0 | 37.9 | 31.5 | 47.2 | 37.9 | 31.6 | 46.7 | 37.3 | 31.6 |
| % Main 2 | 26.8 | 24.7 | 23.3 | 26.8 | 25.0 | 23.8 | 26.7 | 25.0 | 23.5 |
| % Basic | 12.2 | 23.3 | 30.7 | 12.2 | 23.3 | 29.4 | 12.7 | 23.5 | 29.8 |

TABLE 54
Sotatercept Stabilizer Screen (Kleptose in Citrate)

| | 50 mg/mL Sotatercept in 10 mM Citrate, 20 mM L-Methionine, 0.2 mg/mL Polysorbate 80, pH 5.8 | | | | | | | | |
|---|---|---|---|---|---|---|---|---|---|
| | 20 mg/mL Kleptose | | | 5 mg/mL Kleptose | | | 0.5 mg/mL Kleptose | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.60 | 56.60 | 66.95 | 54.73 | 74.74 | 56.10 | 55.27 | 56.59 | 53.75 |
| pH | 5.82 | 5.99 | 6.10 | 5.84 | NT | 5.83 | 5.91 | 5.97 | 5.95 |
| Turbidity A₃₅₀₋₅₀₀ | 0.255 | 0.195 | 0.525 | 0.245 | 0.230 | 0.320 | 0.245 | 0.230 | 0.255 |

UP-SEC

| | | | | | | | | | |
|---|---|---|---|---|---|---|---|---|---|
| % HMW | 1.5 | 3.2 | 4.1 | 1.5 | 3.3 | 4.2 | 1.5 | 3.4 | 4.1 |
| % Monomer | 93.3 | 90.5 | 88.7 | 93.4 | 90.4 | 88.7 | 93.4 | 90.3 | 88.7 |
| % LMW | 5.2 | 6.3 | 7.2 | 5.1 | 6.3 | 7.1 | 5.1 | 6.4 | 7.2 |

cIEF

| | | | | | | | | | |
|---|---|---|---|---|---|---|---|---|---|
| % Acidic | 13.6 | 14.0 | 14.8 | 13.8 | 14.4 | 14.9 | 13.8 | 14.4 | 14.9 |
| % Main 1 | 47.1 | 38.8 | 31.6 | 47.2 | 37.2 | 31.7 | 47.1 | 37.3 | 31.2 |
| % Main 2 | 26.8 | 24.5 | 23.5 | 26.7 | 25.0 | 23.7 | 27.0 | 24.9 | 23.4 |
| % Basic | 12.5 | 22.7 | 30.1 | 12.3 | 23.4 | 29.8 | 12.1 | 23.6 | 30.5 |

TABLE 55
Sotatercept Stabilizer Screen (Proline in Citrate)

| | 50 mg/mL Sotatercept in 10 mM Citrate, 20 mM L-Methionine, 0.2 mg/mL Polysorbate 80, pH 5.8 | | | | | | | | |
|---|---|---|---|---|---|---|---|---|---|
| | 50 mg/mL Proline | | | 5 mg/mL Proline | | | 0.5 mg/mL Proline | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 55.29 | 56.13 | 52.81 | 54.75 | 56.57 | 53.50 | 54.35 | 56.02 | 53.32 |
| pH | 6.02 | 5.98 | 5.96 | 5.97 | 6.08 | 5.84 | 6.20 | 5.90 | 5.81 |
| Turbidity A₃₅₀₋₅₀₀ | 0.215 | 0.205 | 0.190 | 0.200 | 0.160 | 0.220 | 0.235 | 0.205 | 0.190 |

UP-SEC

| | | | | | | | | | |
|---|---|---|---|---|---|---|---|---|---|
| % HMW | 1.5 | 2.9 | 4.2 | 1.5 | 3.2 | 4.2 | 1.5 | 3.3 | 4.2 |
| % Monomer | 93.4 | 90.6 | 88.8 | 93.4 | 90.4 | 88.7 | 93.4 | 90.5 | 88.6 |
| % LMW | 5.1 | 6.4 | 7.0 | 5.1 | 6.3 | 7.2 | 5.1 | 6.2 | 7.3 |

cIEF

| | | | | | | | | | |
|---|---|---|---|---|---|---|---|---|---|
| % Acidic | 14.2 | 13.4 | 13.9 | 14.0 | 13.6 | 14.7 | 14.2 | 14.3 | 15.3 |
| % Main 1 | 46.0 | 36.5 | 31.3 | 47.1 | 39.0 | 31.7 | 46.1 | 37.5 | 30.9 |
| % Main 2 | 27.4 | 24.5 | 22.9 | 26.7 | 24.4 | 23.6 | 26.8 | 25.0 | 23.7 |
| % Basic | 12.4 | 25.6 | 31.9 | 12.2 | 23.00 | 30.0 | 13.0 | 23.1 | 30.1 |

TABLE 56
Sotatercept Stabilizer Screen (Albumin in Citrate)

| | 50 mg/mL Sotatercept in 10 mM Citrate, 20 mM L-Methionine, 0.2 mg/mL Polysorbate 80, pH 5.8 | | | | | | | | |
|---|---|---|---|---|---|---|---|---|---|
| | 40 mg/mL Dextran | | | 10 mg/mL Dextran | | | 0.5 mg/mL Dextran | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 67.54 | 68.94 | 66.95 | 60.56 | 61.94 | 56.10 | 56.25 | 56.61 | 53.75 |
| pH | 6.08 | 6.16 | 6.10 | 6.17 | 6.18 | 5.83 | 6.37 | 5.89 | 5.95 |
| Turbidity A₃₅₀₋₅₀₀ | 0.510 | 0.515 | 0.525 | 0.360 | 0.335 | 0.320 | 0.235 | 0.190 | 0.255 |

UP-SEC

| | | | | | | | | | |
|---|---|---|---|---|---|---|---|---|---|
| % HMW | 3.0 | 4.9 | 3.5 | 2.1 | 3.8 | 4.1 | 1.5 | 3.2 | 4.2 |
| % Monomer | 78.8 | 75.9 | 88.8 | 87.6 | 84.7 | 88.7 | 93.2 | 90.5 | 88.6 |
| % LMW | 18.1 | 19.2 | 7.7 | 10.2 | 11.5 | 7.2 | 5.3 | 6.3 | 7.2 |

cIEF

| | | | | | | | | | |
|---|---|---|---|---|---|---|---|---|---|
| % Acidic | 13.4 | 13.9 | 16.0 | 13.4 | 14.3 | 15.2 | 13.7 | 14.4 | 15.1 |
| % Main 1 | 45.7 | 37.5 | 33.3 | 48.0 | 38.3 | 32.2 | 47.9 | 37.0 | 31.6 |
| % Main 2 | 25.9 | 24.9 | 25.0 | 25.8 | 25.5 | 24.0 | 26.7 | 25.1 | 23.2 |
| % Basic | 15.0 | 23.7 | 25.7 | 12.7 | 21.9 | 28.6 | 11.8 | 23.5 | 30.1 |

TABLE 57
Sotatercept Stabilizer Screen (Controls in Citrate)

| | 50 mg/mL Sotatercept in 10 mM Citrate, 20 mM L-Methionine, 0.2 mg/mL Polysorbate 80, pH 5.8 | | | | | |
|---|---|---|---|---|---|---|
| | No Stabilizer Control | | | 80 mg/mL Sucrose | | |
| | | 50° C. | | | 50° C. | |
| Assay | Initial | 1 W | 2 W | Initial | 1 W | 2 W |
| Conc. (mg/mL) | 55.93 | 56.21 | 52.93 | 50.84 | 57.46 | 57.43 |
| pH | 5.83 | 5.84 | 6.07 | 5.83 | 5.83 | 6.04 |
| Turbidity A₃₅₀₋₅₀₀ | 0.200 | 0.235 | 0.200 | 0.250 | 0.250 | 0.225 |

UP-SEC

| | | | | | | |
|---|---|---|---|---|---|---|
| % HMW | 1.5 | 3.2 | 5.5 | 1.5 | 2.7 | 4.4 |
| % Monomer | 93.3 | 90.5 | 74.8 | 93.4 | 90.9 | 84.2 |
| % LMW | 5.2 | 6.3 | 19.7 | 5.1 | 6.5 | 11.4 |

cIEF

| | | | | | | |
|---|---|---|---|---|---|---|
| % Acidic | 13.6 | 14.0 | 15.1 | 13.5 | 13.9 | 14.4 |
| % Main 1 | 47.0 | 37.7 | 31.6 | 47.3 | 38.0 | 31.8 |
| % Main 2 | 26.9 | 25.1 | 23.6 | 26.8 | 25.1 | 23.6 |
| % Basic | 12.4 | 23.3 | 29.8 | 12.4 | 23.0 | 30.2 |

The results show that under elevated thermal stress the inclusion of the stabilize tested in the formulations provided similar levels of stability to the sotatercept molecule in citrate buffer. Each of the tested formulations maintained high monomer content, and had comparable increases in high molecular weight species over time and comparable levels of charged species compared to samples that did not contain a stabilizer.

TABLE 58
Sotatercept Stabilizer Screen (Polyethylene Glycol - 300 in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 50 mg/mL PEG-300 | | | 25 mg/mL PEG-300 | | | 5 mg/mL PEG-300 | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 50.98 | 52.53 | 51.64 | 50.21 | 52.24 | 51.12 | 50.08 | 51.34 | 51.52 |
| pH | 5.68 | 5.85 | 5.98 | 5.71 | 5.90 | 5.98 | 5.73 | 5.92 | 5.94 |
| Turbidity A₃₅₀₋₅₀₀ | 0.155 | 0.215 | 0.310 | 0.125 | 0.225 | 0.190 | 0.160 | 0.195 | 0.175 |

UP-SEC

| | | | | | | | | | |
|---|---|---|---|---|---|---|---|---|---|
| % HMW | 1.4 | 4.8 | 7.5 | 1.3 | 4.3 | 6.5 | 1.3 | 3.7 | 5.7 |
| % Monomer | 93.1 | 89.1 | 84.6 | 92.9 | 89.6 | 86.1 | 93.0 | 90.0 | 86.8 |
| % LMW | 5.5 | 6.2 | 7.9 | 5.7 | 6.2 | 7.4 | 5.7 | 6.3 | 7.5 |

cIEF

| | | | | | | | | | |
|---|---|---|---|---|---|---|---|---|---|
| % Acidic | 14.6 | 13.4 | 12.9 | 14.8 | 13.3 | 12.5 | 14.6 | 13.1 | 13.2 |
| % Main 1 | 45.2 | 34.1 | 28.6 | 45.7 | 34.8 | 30.0 | 46.4 | 36.0 | 31.6 |
| % Main 2 | 27.4 | 23.7 | 21.5 | 27.4 | 23.7 | 22.2 | 27.2 | 24.2 | 22.6 |
| % Basic | 12.8 | 28.8 | 37.0 | 12.2 | 28.2 | 35.2 | 11.8 | 26.8 | 32.6 |

TABLE 59
Sotatercept Stabilizer Screen (Polyethylene Glycol - 3350 in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 50 mg/mL PEG-3350 | | | 25 mg/mL PEG-3350 | | | 5 mg/mL PEG-3350 | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 54.42 | 54.97 | 54.14 | 51.39 | 52.64 | 51.77 | 50.77 | 51.65 | 51.02 |
| pH | 5.79 | 5.96 | 5.97 | 5.79 | 5.94 | 5.96 | 5.74 | 5.95 | 5.95 |
| Turbidity A₃₅₀₋₅₀₀ | 0.175 | 0.240 | 0.260 | 0.240 | 0.235 | 0.240 | 0.160 | 0.190 | 0.180 |

UP-SEC

| | | | | | | | | | |
|---|---|---|---|---|---|---|---|---|---|
| % HMW | 1.4 | 5.1 | 7.9 | 1.2 | 4.9 | 7.7 | 1.2 | 3.8 | 5.9 |
| % Monomer | 93.0 | 88.7 | 84.8 | 93.1 | 89.2 | 84.8 | 93.5 | 89.4 | 86.7 |
| % LMW | 5.6 | 6.2 | 7.3 | 5.7 | 5.9 | 7.5 | 5.3 | 6.8 | 7.4 |

cIEF

| | | | | | | | | | |
|---|---|---|---|---|---|---|---|---|---|
| % Acidic | 14.5 | 12.9 | 13.1 | 14.6 | 13.3 | 12.8 | 14.5 | 13.0 | 13.3 |
| % Main 1 | 46.4 | 35.6 | 30.3 | 46.1 | 36.5 | 30.5 | 46.1 | 37.2 | 30.4 |
| % Main 2 | 27.7 | 23.6 | 21.9 | 27.4 | 23.1 | 22.1 | 27.6 | 23.1 | 22.7 |
| % Basic | 11.3 | 27.9 | 34.8 | 11.9 | 27.2 | 34.6 | 11.8 | 26.7 | 33.6 |

TABLE 60
Sotatercept Stabilizer Screen (Carboxy Methyl Cellulose (CMC) in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 10 mg/mL CMC | | | 5 mg/mL CMC | | | 1 mg/mL CMC | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 50.58 | 52.13 | 51.72 | 50.82 | 52.09 | 52.22 | 50.40 | 51.63 | 51.62 |
| pH | 5.68 | 5.98 | 6.01 | 5.74 | 6.04 | 6.08 | 5.66 | 6.05 | 6.06 |
| Turbidity A₃₅₀₋₅₀₀ | 0.150 | 0.215 | 0.220 | 0.165 | 0.215 | 0.255 | 0.145 | 0.245 | 0.245 |

UP-SEC

| | | | | | | | | | |
|---|---|---|---|---|---|---|---|---|---|
| % HMW | 1.3 | 6.1 | 8.2 | 1.1 | 6.2 | 8.2 | 1.1 | 5.2 | 6.9 |
| % Monomer | 93.0 | 87.8 | 84.9 | 93.3 | 87.6 | 84.8 | 93.0 | 89.0 | 86.4 |
| % LMW | 5.7 | 6.1 | 6.9 | 5.5 | 6.2 | 7.0 | 5.9 | 5.8 | 6.7 |

cIEF

| | | | | | | | | | |
|---|---|---|---|---|---|---|---|---|---|
| % Acidic | 11.9 | 12.6 | 13.7 | 13.0 | 13.2 | 14.7 | 14.6 | 13.6 | 14.3 |
| % Main 1 | 49.3 | 39.3 | 33.6 | 46.9 | 40.0 | 31.9 | 46.5 | 38.9 | 31.5 |
| % Main 2 | 27.1 | 25.0 | 24.4 | 28.1 | 25.4 | 24.0 | 27.6 | 24.2 | 23.9 |
| % Basic | 11.7 | 23.2 | 28.3 | 12.0 | 21.5 | 29.4 | 11.2 | 23.2 | 30.3 |

TABLE 61
Sotatercept Stabilizer Screen (Dextran in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 20 mg/mL Dextran | | | 10 mg/mL Dextran | | | 5 mg/mL Dextran | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 50.91 | 52.06 | 52.77 | 51.70 | 52.90 | 50.97 | 50.37 | 51.68 | 50.81 |
| pH | 5.70 | 5.92 | 5.99 | 5.72 | 5.96 | 5.98 | 5.75 | 5.97 | 6.01 |
| Turbidity A₃₅₀₋₅₀₀ | 0.145 | 0.210 | 0.240 | 0.205 | 0.210 | 0.210 | 0.125 | 0.125 | 0.200 |

UP-SEC

| | | | | | | | | | |
|---|---|---|---|---|---|---|---|---|---|
| % HMW | 1.2 | 4.7 | 6.9 | 1.4 | 4.2 | 6.1 | 1.3 | 3.9 | 5.6 |
| % Monomer | 93.5 | 89.3 | 86.7 | 93.0 | 89.6 | 86.0 | 93.0 | 89.9 | 87.0 |
| % LMW | 5.4 | 6.0 | 6.4 | 5.6 | 6.1 | 7.9 | 5.7 | 6.2 | 7.4 |

cIEF

| | | | | | | | | | |
|---|---|---|---|---|---|---|---|---|---|
| % Acidic | 14.8 | 13.5 | 14.9 | 14.5 | 13.4 | 13.7 | 14.3 | 13.0 | 13.5 |
| % Main 1 | 45.6 | 37.6 | 32.2 | 46.1 | 36.0 | 31.0 | 45.9 | 37.3 | 31.1 |
| % Main 2 | 27.7 | 23.6 | 17.2 | 27.8 | 24.1 | 22.7 | 27.9 | 23.8 | 22.7 |
| % Basic | 12.0 | 25.3 | 35.7 | 11.6 | 26.3 | 32.6 | 11.8 | 25.9 | 32.7 |

TABLE 62
Sotatercept Stabilizer Screen (Dextrose in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 80 mg/mL Dextrose | | | 40 mg/mL Dextrose | | | 5 mg/mL Dextrose | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 50.47 | 50.76 | 51.59 | 50.34 | 51.75 | 50.81 | 50.59 | 52.46 | 50.81 |
| pH | 5.62 | 5.96 | 6.00 | 5.64 | 5.91 | 5.98 | 5.64 | 6.97 | 5.99 |
| Turbidity A₃₅₀₋₅₀₀ | 0.175 | 0.385 | 0.920 | 0.140 | 0.305 | 0.580 | 0.120 | 0.210 | 0.235 |

UP-SEC

| | | | | | | | | | |
|---|---|---|---|---|---|---|---|---|---|
| % HMW | 1.1 | 3.1 | 6.7 | 1.2 | 3.2 | 5.7 | 1.3 | 3.6 | 5.4 |
| % Monomer | 93.6 | 90.6 | 86.1 | 93.7 | 90.9 | 86.1 | 93.0 | 90.2 | 87.2 |
| % LMW | 5.3 | 6.3 | 7.3 | 5.1 | 5.8 | 8.2 | 5.6 | 6.2 | 7.4 |

cIEF

| | | | | | | | | | |
|---|---|---|---|---|---|---|---|---|---|
| % Acidic | 15.1 | 17.9 | 25.8 | 14.9 | 15.8 | 19.9 | 14.8 | 13.6 | 14.2 |
| % Main 1 | 45.6 | 34.0 | 23.9 | 45.3 | 36.0 | 28.1 | 45.6 | 36.0 | 30.9 |
| % Main 2 | 27.5 | 30.6 | 30.4 | 27.6 | 28.2 | 28.8 | 27.4 | 24.5 | 23.5 |
| % Basic | 11.8 | 17.4 | 20.0 | 12.2 | 19.9 | 23.3 | 12.36 | 26.0 | 31.3 |

TABLE 63
Sotatercept Stabilizer Screen (Mannitol in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 80 mg/mL Mannitol | | | 40 mg/mL Mannitol | | | 5 mg/mL Mannitol | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 52.97 | 53.60 | 51.82 | 50.61 | 51.67 | 50.80 | 50.01 | 51.36 | 51.40 |
| pH | 5.61 | 5.89 | 6.01 | 5.72 | 5.91 | 6.03 | 5.69 | 5.97 | 6.11 |
| Turbidity A₃₅₀₋₅₀₀ | 0.145 | 0.200 | 0.195 | 0.125 | 0.165 | 0.180 | 0.120 | 0.150 | 0.175 |

UP-SEC

| | | | | | | | | | |
|---|---|---|---|---|---|---|---|---|---|
| % HMW | 1.2 | 2.7 | 3.7 | 1.3 | 3.0 | 4.2 | 1.1 | 3.2 | 5.2 |
| % Monomer | 93.3 | 961.1 | 89.2 | 93.0 | 90.7 | 89.0 | 93.4 | 90.5 | 87.5 |
| % LMW | 5.5 | 6.2 | 7.1 | 5.7 | 6.3 | 6.8 | 5.5 | 6.3 | 7.4 |

cIEF

| | | | | | | | | | |
|---|---|---|---|---|---|---|---|---|---|
| % Acidic | 14.7 | 13.3 | 13.2 | 14.9 | 13.2 | 13.6 | 14.7 | 12.8 | 13.4 |
| % Main 1 | 45.8 | 36.4 | 31.4 | 45.5 | 36.3 | 30.5 | 45.9 | 37.9 | 30.9 |
| % Main 2 | 27.5 | 24.2 | 22.9 | 27.6 | 24.4 | 22.8 | 27.7 | 23.8 | 23.1 |
| % Basic | 12.0 | 26.0 | 32.5 | 12.0 | 26.1 | 33.0 | 11.7 | 25.4 | 32.7 |

TABLE 64
Sotatercept Stabilizer Screen (Trehalose in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 80 mg/mL Trehalose | | | 40 mg/mL Trehalose | | | 5 mg/mL Trehalose | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 50.61 | 51.97 | 51.38 | 50.18 | 56.08 | 53.41 | 50.13 | 51.22 | 50.50 |
| pH | 5.58 | 5.89 | 6.02 | 5.62 | 5.90 | 6.05 | 5.67 | 5.90 | 6.05 |
| Turbidity A₃₅₀₋₅₀₀ | 0.160 | 0.190 | 0.190 | 0.160 | 0.225 | 0.250 | 0.165 | 0.190 | 0.195 |

UP-SEC

| | | | | | | | | | |
|---|---|---|---|---|---|---|---|---|---|
| % HMW | 1.1 | 2.5 | 3.7 | 1.1 | 2.9 | 4.3 | 1.2 | 3.5 | 5.0 |
| % Monomer | 93.2 | 91.5 | 89.5 | 93.4 | 91.1 | 89.0 | 93.6 | 90.7 | 88.1 |
| % LMW | 5.7 | 6.0 | 6.8 | 5.5 | 6.0 | 6.7 | 5.2 | 5.9 | 7.0 |

cIEF

| | | | | | | | | | |
|---|---|---|---|---|---|---|---|---|---|
| % Acidic | 14.5 | 13.7 | 13.6 | 14.6 | 13.1 | 13.4 | 14.9 | 13.2 | 13.5 |
| % Main 1 | 45.4 | 36.5 | 30.7 | 45.5 | 36.5 | 31.8 | 45.7 | 36.2 | 31.6 |
| % Main 2 | 27.4 | 24.4 | 23.0 | 27.8 | 24.7 | 23.1 | 27.6 | 24.4 | 23.0 |
| % Basic | 12.6 | 25.4 | 32.6 | 12.1 | 25.7 | 31.7 | 11.7 | 26.2 | 31.9 |

TABLE 65
Sotatercept Stabilizer Screen (Kleptose in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 20 mg/mL Kleptose | | | 5 mg/mL Kleptose | | | 0.5 mg/mL Kleptose | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 50.55 | 51.82 | 50.63 | 50.18 | 51.24 | 50.10 | 50.51 | 51.64 | 50.68 |
| pH | 5.64 | 5.98 | 5.98 | 5.75 | 5.93 | 5.94 | 5.77 | 2.94 | 5.98 |
| Turbidity A₃₅₀₋₅₀₀ | 0.135 | 0.205 | 0.190 | 0.165 | 0.190 | 0.145 | 0.140 | 0.200 | 0.200 |

UP-SEC

| | | | | | | | | | |
|---|---|---|---|---|---|---|---|---|---|
| % HMW | 1.3 | 3.5 | 5.4 | 1.3 | 3.6 | 5.4 | 1.3 | 3.7 | 5.3 |
| % Monomer | 93.0 | 90.7 | 87.4 | 93.0 | 90.5 | 87.8 | 93.0 | 90.4 | 87.2 |
| % LMW | 5.6 | 5.8 | 7.2 | 5.7 | 5.9 | 6.8 | 5.7 | 6.0 | 7.5 |

cIEF

| | | | | | | | | | |
|---|---|---|---|---|---|---|---|---|---|
| % Acidic | 14.6 | 12.6 | 14.4 | 15.0 | 13.0 | 13.9 | 14.8 | 13.4 | 14.1 |
| % Main 1 | 45.8 | 37.6 | 33.1 | 45.6 | 37.5 | 31.7 | 45.6 | 35.8 | 31.4 |
| % Main 2 | 27.6 | 23.8 | 17.1 | 27.3 | 23.5 | 18.2 | 27.4 | 24.0 | 18.4 |
| % Basic | 12.0 | 26.0 | 35.4 | 12.1 | 26.0 | 36.2 | 12.2 | 26.8 | 36.1 |

TABLE 66
Sotatercept Stabilizer Screen (Proline in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 50 mg/mL Proline | | | 5 mg/mL Proline | | | 0.5 mg/mL Proline | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 5.045 | 52.14 | 51.17 | 49.95 | 50.85 | 50.38 | 49.96 | 51.21 | 50.71 |
| pH | 5.76 | 6.00 | 6.02 | 5.83 | 5.88 | 5.91 | 5.72 | 5.88 | 6.01 |
| Turbidity A₃₅₀₋₅₀₀ | 0.095 | 0.200 | 0.205 | 0.130 | 0.125 | 0.155 | 0.135 | 0.175 | 0.175 |

UP-SEC

| | | | | | | | | | |
|---|---|---|---|---|---|---|---|---|---|
| % HMW | 1.1 | 2.7 | 3.8 | 1.1 | 3.4 | 5.1 | 1.1 | 3.6 | 5.4 |
| % Monomer | 93.3 | 90.8 | 88.6 | 93.2 | 90.1 | 87.0 | 93.6 | 90.5 | 87.2 |
| % LMW | 5.6 | 6.5 | 7.7 | 5.7 | 6.4 | 7.9 | 5.3 | 5.9 | 7.4 |

cIEF

| | | | | | | | | | |
|---|---|---|---|---|---|---|---|---|---|
| % Acidic | 14.9 | 13.1 | 14.1 | 14.8 | 13.1 | 13.4 | 14.9 | 12.9 | 14.3 |
| % Main 1 | 45.7 | 37.4 | 31.0 | 45.7 | 37.5 | 32.1 | 45.7 | 36.8 | 32.7 |
| % Main 2 | 27.7 | 24.0 | 18.5 | 27.3 | 23.8 | 18.7 | 27.3 | 24.1 | 18.3 |
| % Basic | 11.8 | 25.5 | 36.3 | 12.2 | 25.6 | 35.9 | 12.1 | 26.2 | 34.7 |

TABLE 67
Sotatercept Stabilizer Screen (Albumin in Histidine)

| | 50 mg/mL Sotatercept in10 mM Histidine, 20 mM L-Methionine, 0.2 mg/mL Polysorbate 80, pH 5.8 | | | | | | | | |
|---|---|---|---|---|---|---|---|---|---|
| | 40 mg/mL Dextran | | | 10 mg/mL Dextran | | | 0.5 mg/mL Dextran | | |
| | | 50° C. | | | 50° C. | | | 50° C. | |
| Assay | Initial | 1 W | 2 W | Initial | 1 W | 2 W | Initial | 1 W | 2 W |
| Conc. (mg/mL) | 61.07 | 63.92 | 62.30 | 53.20 | 55.11 | 54.27 | 50.06 | 51.12 | 51.18 |
| pH | 5.88 | 6.15 | 6.15 | 5.75 | 6.03 | 6.05 | 5.71 | 6.01 | 6.04 |
| Turbidity A₃₅₀₋₅₀₀ | 0.370 | 0.500 | 0.510 | 0.220 | 0.260 | 0.290 | 0.165 | 0.195 | 0.220 |

UP-SEC

| | | | | | | | | | |
|---|---|---|---|---|---|---|---|---|---|
| % HMW | 2.6 | 5.5 | 6.7 | 1.3 | 3.1 | 4.6 | 1.2 | 3.6 | 5.3 |
| % Monomer | 80.4 | 77.0 | 75.9 | 90.0 | 87.4 | 85.0 | 94.0 | 89.9 | 87.3 |
| % LMW | 17.0 | 17.5 | 17.4 | 8.7 | 9.5 | 10.5 | 4.9 | 6.5 | 7.4 |

cIEF

| | | | | | | | | | |
|---|---|---|---|---|---|---|---|---|---|
| % Acidic | 14.0 | 14.3 | 14.5 | 14.7 | 13.6 | 15.2 | 14.7 | 13.5 | 13.9 |
| % Main 1 | 45.1 | 38.1 | 28.8 | 45.0 | 37.4 | 28.7 | 46.0 | 36.3 | 31.8 |
| % Main 2 | 26.4 | 24.7 | 21.7 | 27.8 | 24.4 | 21.9 | 27.8 | 24.0 | 18.6 |
| % Basic | 14.5 | 22.8 | 35.1 | 12.5 | 24.6 | 34.2 | 11.5 | 26.2 | 35.7 |

TABLE 68
Sotatercept Stabilizer Screen (Controls in Histidine)

| | 50 mg/mL Sotatercept in10 mM Histidine, 20 mM L-Methionine, 0.2 mg/mL Polysorbate 80, pH 5.8 | | | | | |
|---|---|---|---|---|---|---|
| | No Stabilizer Control | | | 80 mg/mL Sucrose | | |
| | | 50° C. | | | 50° C. | |
| Assay | Initial | 1 W | 2 W | Initial | 1 W | 2 W |
| Conc. (mg/mL) | 48.89 | 50.89 | 50.54 | 51.58 | 53.36 | 52.90 |
| pH | 5.76 | 6.07 | 6.08 | 5.88 | 5.90 | 5.95 |
| Turbidity A₃₅₀₋₅₀₀ | 0.135 | 0.185 | 0.220 | 0.165 | 0.195 | 0.245 |

UP-SEC

| | | | | | | |
|---|---|---|---|---|---|---|
| % HMW | 1.1 | 3.6 | 5.3 | 1.1 | 2.7 | 3.6 |
| % Monomer | 93.3 | 90.3 | 87.2 | 93.2 | 91.5 | 89.2 |
| % LMW | 5.6 | 6.1 | 7.5 | 5.6 | 5.8 | 7.2 |

cIEF

| | | | | | | |
|---|---|---|---|---|---|---|
| % Acidic | 14.5 | 13.2 | 14.1 | 15.0 | 13.5 | 13.7 |
| % Main 1 | 45.5 | 37.9 | 31.6 | 45.6 | 36.3 | 31.8 |
| % Main 2 | 27.8 | 23.6 | 18.4 | 28.2 | 24.6 | 19.8 |
| % Basic | 12.2 | 25.3 | 35.9 | 11.2 | 25.6 | 34.7 |

The results show that under elevated thermal stress the inclusion of the stabilizers tested in the formulations provided similar levels of stability to the sotatercept molecule in histidine buffer. Each of the tested formulations maintained high monomer contents, and had comparable increases in high molecular weight species over time and comparable levels of charged species.

### Sotatercept Formulations

Tables 69-71 show liquid pharmaceutical formulations containing sotatercept (SEQ ID NO: 32, SEQ ID NO: 41 or a mixture of both SEQ ID NO: 32 and SEQ ID NO: 41) that were prepared:

TABLE 69

| | | |
|---|---|---|
| Human ActRIIA fusion | 50 | mg/mL |
| protein SEQ ID NO: 32 | | |
| citrate buffer | 10 | mM |
| Sucrose | 8% | weight/volume |
| polysorbate 80 | 0.2 | mg/mL |
| L-methionine | 20 | mM |

TABLE 70

| | | |
|---|---|---|
| Human ActRIIA fusion | 50 | mg/mL |
| protein SEQ ID NO: 41 | | |
| citrate buffer | 10 | mM |
| Sucrose | 8% | weight/volume |
| polysorbate 80 | 0.2 | mg/mL |
| L-methionine | 20 | mM |

TABLE 71

| | | |
|---|---|---|
| Human ActRIIA fusion protein SEQ | 50 | mg/mL |
| ID NO: 32 and SEQ ID NO: 41 | | |
| citrate buffer | 10 | mM |
| Sucrose | 8% | weight/volume |
| polysorbate 80 | 0.2 | mg/mL |
| L-methionine | 20 | mM |

### SEQUENCES

TABLE 72
Sequences

| | |
|---|---|
| SEQ ID NO: 9 | MGAAAKLAFAVFLISCSSGAILGRSETQECLFFNANWEKDRTNQTGVEPCYGDKDKRRHC |
| | FATWKNISGSIEIVKQGCWLDDINCYDRTDCVEKKDSPEVYFCCCEGNMCNEKFSYFPEME |
| | VTQPTSNPVTPKPPYYNILLYSLVPLMLIAGIVICAFWVYRHHKMAYPPVLVPTQDPGPPPP |
| | SPLLGLKPLQLLEVKARGRFGCVWKAQLLNEYVAVKIFPIQDKQSWQNEYEVYSLPGMKH |
| | ENILQFIGAEKRGTSVDVDLWLITAFHEKGSLSDFLKANVVSWNELCHIAETMARGLAYLH |
| | EDIPGLKDGHKPAISHRDIKSKNVLLKNNLTACIADFGLALKFEAGKSAGDTHGQVGTRRY |
| | MAPEVLEGAINFQRDAFLRIDMYAMGLVLWELASRCTAADGPVDEYMLPFEEEIGQHPSL |
| | EDMQEVVVHKKKRPVLRDYWQKHAGMAMLCETIEECWDHDAEARLSAGCVGERITQMQ |
| | RLTNIITTEDIVTVVTMVTNVDFPPKESSL |
| SEQ ID NO: 10 | ILGRSETQECLFFNANWEKDRTNQTGVEPCYGDKDKRRHCFATWKNISGSIEIVKQGCWLD |
| | DINCYDRTDCVEKKDSPEVYFCCCEGNMCNEKFSYFPEMEVTQPTSNPVTPKPP |
| SEQ ID NO: 11 | ILGRSETQECLFFNANWEKDRTNQTGVEPCYGDKDKRRHCFATWKNISGSIEIVKQGCWLD |
| | DINCYDRTDCVEKKDSPEVYFCCCEGNMCNEKFSYFPEM |
| SEQ ID NO: 12 | ATGGGAGCTGCTGCAAAGTTGGCGTTTGCCGTCTTTCTTATCTCCTGTTCTTCAGGTGCT |
| | ATACTTGGTAGATCAGAAACTCAGGAGTGTCTTTTCTTTAATGCTAATTGGGAAAAAGA |
| | CAGAACCAATCAAACTGGTGTTGAACCGTGTTATGGTGACAAAGATAAACGGCGGCAT |
| | TGTTTTGCTACCTGGAAGAATATTTCTGGTTCCATTGAAATAGTGAAACAAGG |
| | TTGTTGGCTGGATGATATCAACTGCTATGACAGGACTGAT |
| | TGTGTAGAAAAAAAAGACAGCCCTGAAGTATATTTTTGTTGCTGTGAGGG |
| | CAATATGTGTAATGAAAAGTTTTCTTATTTTCCGGAGATG |
| | GAAGTCACACAGCCCACTTCAAATCCAGTTACACCTAAGCCACCCTATTA |
| | CAACATCCTGCTCTATTCCTTGGTGCCACTTATGTTAATT |
| | GCGGGGATTGTCATTTGTGCATTTTGGGTGTACAGGCATCACAAGATGGCCTACCCTCC |
| | TGTACTTGTTCCAACTCAAGACCCAGGACCACCCCCACCTTCTCCATTACTAGGTTTGA |
| | AACCACTGCAGTTATTAGAAGTGAAAGCAAGGGGAAGATTTGGTTGTGTCTGGAAAGC |
| | CCAG |
| | TTGCTTAACGAATATGTGGCTGTCAAAATATTTCCAATACAGGACAAACAGTCATGGCA |
| | AAATGAATACGAAGTCTACAGTTTGCCTGGA |
| | ATGAAGCATGAGAACATATTACAGTTCATTGGTGCAGAAAAACGAGGCACCAGTGTTG |
| | ATGTGGATCTTTGGCTGATCACAGCATTTCATGAAAAGGGTTCACTATCAGACTTTCTT |
| | AAGGCTAATGTGGTCTCTTGGAATGAACTGTGTCATATTGCAGAAACCATGGCTAGAG |
| | GATTGGCATATTTACATGAGGATATACCTGGCCTAAAAGATGGCC |
| | ACAAACCTGCCATATCTCACAGGGACATCAAAAGTAAAAATGTGCTGTTGAAAAACAA |
| | CCTGACAGCTTGCATTGCTGACTTTGGGTTGGCCTTAAAATTTGAGGCTGGCAAGTCTG |
| | CAGGCGATACCCATGGACAGGTTGGTACCCGGAGGTACATGGCTCCAGAGGTATTAGA |
| | GGGTGCTATAAACTTCCAAAGGGATGCATTTTTGAGGATAGATATGTATGCCATGGGAT |
| | TAGTCCTATGGGAACTGGCTTCTCGCTGTACTGCTGCAGATGGACCTGTAGATGAATAC |
| | ATGTTGCCATTTGAGGAGGAAATTGGCCAGCATCCATCTCTTGAAGACATGCAGGAAG |
| | TTGTTGTGCATAAAAAAAAGAGGCCTGTTTTAAGAGATTATTGGCAGAAACATGCTGG |
| | AATGGCAATGCTCTGTGAAACCATTGAAGAATGTTGGGATCACGACGCAGAAGCCAGG |
| | TTATCAGCTGGATGTGTAGGTGAAAGAATTACCCAGATGCAGAGACTAACAAATATTA |
| | TTACCACAGAGGACATTGTAACAGTGGTCACAATGGTGACAAATGTTGACTTTCCTCCC |
| | AAAGAATCTAGTCTA |
| SEQ ID NO: 13 | ATACTTGGTAGATCAGAAACTCAGGAGTGTCTTTTCTTTAATGCTAATTGGGAAAAAGA |
| | CAGAACCAATCAAACTGGTGTTGAACCGTGTTATGGTGACAAAGATAAACGGCGGCAT |
| | TGTTTTGCTACCTGGAAGAATAT |
| | TTCTGGTTCCATTGAAATAGTGAAACAAGGTTGTTGGCTG |
| | GATGATATCAACTGCTATGACAGGACTGATTGTGTAGAAAAAAAAGACAGCCCTGAAG |
| | TATATTTTTGTTGCTGTGAGGGCAATATGTGTAATGAAAAGTTTTCTTATTTTCCGGAGA |
| | TGGAAGTCACACAGCCCACTTCAAATCCAGTTACACCTAAGCCACCC |
| SEQ ID NO: 32 | ILGRSETQECLFFNANWEKDRTNQTGVEPCYGDKDKRRHCFATWKNISGSIEIVKQGCWLD |
| | DINCYDRTDCVEKKDSPEVYFCCCEGNMCNEKFSYFPEMEVTQPTSNPVTPKPPTGGGTHT |
| | CPPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPVPIEKTISKAKGQPREP |
| | QVYTLPPSREEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 41 | ILGRSETQECLFFNANWEKDRTNQTGVEPCYGDKDKRRHCFATWKNISGSIEIVKQGCWLD |
| | DINCYDRTDCVEKKDSPEVYFCCCEGNMCNEKFSYFPEMEVTQPTSNPVTPKPPTGGGTHT |
| | CPPCPAPELLGGPSVFLFPPKPKDTLMISRTPEVTCVVVDVSHEDPEVKFNWYVDGVEVHN |
| | AKTKPREEQYNSTYRVVSVLTVLHQDWLNGKEYKCKVSNKALPVPIEKTISKAKGQPREP |
| | QVYTLPPSREEMTKNQVSLTCLVKGFYPSDIAVEWESNGQPENNYKTTPPVLDSDGSFFLY |
| | SKLTVDKSRWQQGNVFSCSVMHEALHNHYTQKSLSLSPG |
| SEQ ID NO: 19 | GGG |
| SEQ ID NO: 20 | GGGG |
| SEQ ID NO: 21 | TGGGG |
| SEQ ID NO: 22 | SGGGG |
| SEQ ID NO: 23 | TGGG |
| SEQ ID NO: 24 | SGGG |
| SEQ ID NO: 25 | GGGGS |
| SEQ ID NO: 33 | MKFLVNVALVFMVVYISYIYA |
| SEQ ID NO: 34 | MDAMKRGLCCVLLLCGAVFVSP |
| SEQ ID NO: 35 | MGAAAKLAFAVFLISCSSGA |
| SEQ ID NO: 36 | MDAMKRGLCCVLLLCGAVFVSPGAAILGRSETQECLFFNANWEKDRTNQTGVEPCYGDK |
| | DKRRHCFATWKNISGSIEIVKQGCWLDDINCYDRTDCVEKKDSPEVYFCCCEGNMCNEKF |
| | SYFPEMEVTQPTSNPVTPKPPTGGGTHTCPPCPAPELLGGPSVFLFPPKPKDTLMISRTPEVT |
| | CVVVDVSHEDPEVKFNWYVDGVEVHNAKTKPREEQYNSTYRVVSVLTVLHQDWLNGKE |
| | YKCKVSNKALPVPIEKTISKAKGQPREPQVYTLPPSREEMTKNQVSLTCLVKGFYPSDIAVE |
| | WESNGQPENNYKTTPPVLDSDGSFFLYSKLTVDKSRWQQGNVFSCSVMHEALHNHYTQK |
| | SLSLSPGK |
| SEQ ID NO: 37 | ATGGATGCAATGAAGAGAGGGCTCTGCTGTGTGCTGCTGCTGTGTGGAGCAGTCTTCGT |
| | TTCGCCCGGCGCCGCTATACTTGGTAGATCAGAAACTCAGGAGTGTCTTTTTTTAATGC |
| | TAATTGGGAAAAAGACAGAACCAATCAAACTGGTGTTGAACCGTGTTATGGTGACAAA |
| | GATAAACGGCGGCATTGTTTTGCTACCTGGAAGAATATTTCTGGTTCCATTGAATAGTG |
| | AAACAAGGTTGTTGGCTGGATGATATCAACTGCTATGACAGGACTGATTGTGTAGAAA |
| | AAAAAGACAGCCCTGAAGTATATTTCTGTTGCTGTGAGGGCAATATGTGTAATGAAAA |
| | GTTTTCTTATTTTCCGGAGATGGAAGTCACACAGCCCACTTCAAATCCAGTTACACCTA |
| | AGCCACCCACCGGTGGTGGAACTCACACATGCCCACCGTGCCCAGCACCTGAACTCCT |
| | GGGGGGACCGTCAGTCTTCCTCTTCCCCCCAAAACCCAAGGACACCCTCATGATCTCCC |
| | GGACCCCTGAGGTCACATGCGTGGTGGTGGACGTGAGCCACGAAGACCCTGAGGTCAA |
| | GTTCAACTGGTACGTGGACGGCGTGGAGGTGCATAATGCCAAGACAAAGCCGCGGGAG |
| | GAGCAGTACAACAGCACGTACCGTGTGGTCAGCGTCCTCACCGTCCTGCACCAGGACT |
| | GGCTGAATGGCAAGGAGTACAAGTGCAAGGTCTCCAACAAAGCCCTCCCAGTCCCCAT |
| | CGAGAAAACCATCTCCAAAGCCAAAGGGCAGCCCCGAGAACCACAGGTGTACACCCTG |
| | CCCCCATCCCGGGAGGAGATGACCAAGAACCAGGTCAGCCTGACCTGCCTGGTCAAAG |
| | GCTTCTATCCCAGCGACATCGCCGTGGAGTGGGAGAGCAATGGGCAGCCGGAGAACAA |
| | CTACAAGACCACGCCTCCCGTGCTGGACTCCGACGGCTCCTTCTTCCTCTATAGCAAGC |
| | TCACCGTGGACAAGAGCAGGTGGCAGCAGGGGAACGTCTTCTCATGCTCCGTGATGCA |
| | TGAGGCTCTGCACAACCACTACACGCAGAAGAGCCTCTCCCTGTCTCCGGGTAAATGA |
| | GAATTC |
| SEQ ID NO: 38 | ILGRSETQE |
| SEQ ID NO: 62 | ILGRSETQECIFYNANWERDRTNRTGVESCYGDKDKRRHCFATWKNISGSIEIVKQGCWLD |
| | DINCYDRTDCIEKKDSPEVYFCCCEGNMCNERFSYFPEMEVTQPTSNPVTPKPP |
| SEQ ID NO: 65 | ILGRSETQECIYYNANWEKDKTNRSGIEPCYGDKDKRRHCFATWKNISGSIEIVKQGCWLD |
| | DINCYDRNDCIEKKDSPEVFFCCCEGNMCNERFFYFPEMEVTQPTSNPVTPKPP |
| SEQ ID NO: 66 | ILGRSETQECIFYNANWERDRTNRTGVESCYGDKDKRRHCFATWKNISGSIEIVKQGCWLD |
| | DINCYDRTDCIEKKDSPEVYFCCCEGNMCNERFSYFPEMEVTQPTSNPVTPKPP |
| SEQ ID NO: 67 | ILGRSETQECIYYNANWEKDKTNRSGIEPCYGDKDKRRHCFATWKNISGSIEIVKQGCWLD |
| | DINCYDRNDCIEKKDSPEVFFCCCEGNMCNERFFYFPEMEVTQPTSNPVTPKP |
| SEQ ID NO: 68 | ILGRSETQECIFYNANWERDKTNRTGVELCYGDKDKRRHCFATWKNISGSIEIVKQGCWLD |
| | DINCYDRTDCIEKKDSPEVYFCCCEGNMCNERFSYFPEMEVTQPTSNPVTPKP |

Exemplary methods and materials are described herein, although methods and materials similar or equivalent to those described herein can also be used in the practice or testing of the presently disclosed methods and formulations. All publications, patent applications, patents, and other references mentioned herein are incorporated by reference in their entirety.

We claim:

1. A pharmaceutical formulation comprising 50-100 mg/mL of a human ActRIIa fusion protein comprising the amino acid sequence of SEQ ID NO: 32, or SEQ ID NO: 41, a buffer, a surfactant, a stabilizing agent, and optionally one or more antioxidants; wherein the buffer is not histidine.

2. The pharmaceutical formulation of claim 1, wherein the fusion protein has a concentration of 100 mg/mL.

3. The pharmaceutical formulation of claim 1, wherein the fusion protein has a concentration of 50 mg/mL.

4. The pharmaceutical formulation of anyone of claims 1-3, wherein the buffer is selected from the group consisting of succinate, phosphate, acetate, citrate, and glutamate.

5. The pharmaceutical formulation of claim 4, wherein the buffer is a phosphate buffer that maintains a pH of the formulation between 5-7.

6. The pharmaceutical formulation of claim 4, wherein the buffer is a citrate buffer that maintains a pH of the formulation between 4.5-7.

7. The pharmaceutical formulation of claim 4, wherein the buffer is an acetate buffer that maintains a pH of the formulation between 4.5-6.

8. The pharmaceutical formulation of claim 4, wherein the buffer is a succinate buffer that maintains a pH of the formulation between 4.5-5.

9. The pharmaceutical formulation of anyone of claims 1-3, wherein the buffer is a glutamate buffer that maintains a pH of the formulation between 5-7.

10. The pharmaceutical formulation of any one of claims 1-9, wherein the buffer maintains a pH of 5.8 of the formulation.

11. The pharmaceutical formulation of any one of claims 1-10, wherein the buffer is present at a concentration between 10-50 mM.

12. The pharmaceutical formulation of any one of claims 1-10, wherein the buffer is present at a concentration of at least 10 mM.

13. The pharmaceutical formulation of claim 12, wherein the stabilizing agent is sucrose.

14. The pharmaceutical formulation of any one of claims 1-13, wherein the stabilizing agent is present at a concentration between 2-16% weight/volume.

15. The pharmaceutical formulation of any one of claims 1-14, wherein the stabilizing agent is present at a concentration between 6-10% weight/volume.

16. The pharmaceutical formulation of any one of claims 1-15, wherein the stabilizing agent is present at a concentration of at least 8% weight/volume.

17. The pharmaceutical formulation of any one of claims 1-16, wherein the surfactant is selected from the group consisting of polysorbate 20, polysorbate 80, poloxamer 124, poloxamer 127, poloxamer 188, and poloxamer 407.

18. The pharmaceutical formulation of claim 17, wherein the surfactant is polysorbate 80.

19. The pharmaceutical formulation of claim 17, wherein the surfactant is polysorbate 20.

20. The pharmaceutical formulation of claim 17, wherein the surfactant is poloxamer 188.

21. The pharmaceutical formulation of any one of claims 1-20, wherein the surfactant is present at a concentration between 0.05-0.5 mg/ml.

22. The pharmaceutical formulation of any one of claims 1-20, wherein the surfactant is present at a concentration 0.1-0.5 mg/ml.

23. The pharmaceutical formulation of any one of claims 1-22, wherein the surfactant is present at a concentration of at least 0.2 mg/ml.

24. The pharmaceutical formulation of any one of claims 1-23, wherein the pharmaceutical formulation further comprises an antioxidant.

25. The pharmaceutical formulation of any one of claims 1-24, wherein the pharmaceutical formulation further comprises a chelating agent, wherein the chelating agent is DTPA or EDTA.

26. The pharmaceutical formulation of claim 25, wherein the chelating agent is present at a concentration between 7.5-100 μM.

27. The pharmaceutical formulation of any one of claims 25-26, wherein the chelating agent is present at a concentration of 10 μM.

28. The pharmaceutical formulation of claim 24, wherein the antioxidant is methionine.

29. The pharmaceutical formulation of claim 28, wherein the antioxidant is L-methionine.

30. The pharmaceutical formulation of any one of claims 28-29, wherein the antioxidant is present at a concentration of between 7.5-50 mM.

31. The pharmaceutical formulation of any one of claims 28-29, wherein the antioxidant is present at a concentration between 5-20 mM.

32. A liquid pharmaceutical formulation comprising 50-100 mg/mL of a human ActRIIa fusion protein comprising the amino acid sequence of SEQ ID NO: 32 or SEQ ID NO:41, 10-50 mM of citrate buffer, 2-16% weight/volume of sucrose, 0.05-0.5 mg/mL of polysorbate 80, polysorbate 20 or polaxamer 188, 0-50 mM of L-methionine and 0-100 μM of DTPA or EDTA.

33. A liquid pharmaceutical formulation comprising 50 mg/mL of a human ActRIIa fusion protein comprising the amino acid sequence of SEQ ID NO: 32 or SEQ ID NO:41, 10 mM of citrate buffer, 8% weight/volume of sucrose, 0.2 mg/mL of polysorbate 80, and 20 mM of L-methionine.

34. The pharmaceutical formulation of any one of claims 1-33, wherein the pharmaceutical formulation is for the treatment of pulmonary arterial hypertension in a subject in need thereof.

35. The pharmaceutical formulation any one of claims 1-34, wherein the pharmaceutical formulation is administered via autoinjector.

36. The pharmaceutical formulation any one of claims 1-35, wherein the pharmaceutical formulation is administered via subcutaneous injection.

37. The pharmaceutical formulation any one of claims 1-35, wherein the pharmaceutical formulation is a liquid formulation.

38. The pharmaceutical formulation any one of claims 1-35, wherein the pharmaceutical formulation is stable when stored between 2-8° C. for at least one month.

39. The pharmaceutical formulation of any of claims 1-38, wherein the formulation is contained in an injection device.

40. The pharmaceutical formulation of claim 39, wherein the injection device is an autoinjector.

41. The pharmaceutical formulation of any of claims 1-38, wherein the formulation is contained in a glass vial.

42. A method of treating pulmonary arterial hypertension (PAH) in a human patient in need thereof, comprising administering to the patient a pharmaceutical formulation of any one of claims 1-41.

43. Use of the pharmaceutical formulation of any one of claims 1-41, for the treatment of pulmonary arterial hypertension (PAH) in a patient in need thereof.
